(19) United States
(12) Patent Application Publication
(10) Pub. No.: US 2021/0283001 A1
(43) Pub. Date: Sep. 16, 2021

(54) APPARATUS COMPRISING A SUPPORT SYSTEM FOR A USER AND ITS OPERATION IN A GRAVITY-ASSIST MODE

(71) Applicant: ECOLE POLYTECHNIQUE FEDERALE DE LAUSANNE (EPFL), Lausanne (CH)

(72) Inventors: Joachim VON ZITZEWITZ, Lausanne (CH); Jean Baptiste MIGNARDOT, ST-SULPICE VD (CH); Camille Georgette Marie LE GOFF, EPALINGES (CH); Grégoire COURTINE, Lausanne (CH); Heike VALLERY, CN DELFT (NL); Michiel Plooij, CN DELFT (NL)

(21) Appl. No.: 16/326,593

(22) PCT Filed: Aug. 17, 2017
(86) PCT No.: PCT/EP2017/070822

§ 371 Date: Feb. 19, 2019

(30) Foreign Application Priority Data
Aug. 17, 2016 (EP) 16184544.1

(51) Int. Cl. A61H 3/00; A61N 1/05; A61N 1/36; B25J 9/00

(52) U.S. Cl. CPC A61H 3/00; A61N 1/0551; A61N 1/36003; A61N 1/36067; B25J 9/0006; A61H 2201/10; A61H 2201/1666; A61H 2201/5007; A61H 2201/5061; A61H 2201/5069; A61H 2201/165

(57) ABSTRACT

The present application relates to devices and systems for rehabilitation of the locomotor system, for example limbs. In particular, the present application discloses an apparatus, more in particular a robotic platform capable of optimizing gravity-dependent trunk movements, enabling overground locomotion in non-ambulatory individuals with spinal cord injury and stroke, while promoting durable motor improvement when delivered during gait rehabilitation facilitated by electrical spinal cord stimulation.

The present application relates to the field of locomotion control and medical engineering, and in some examples to devices, methods and/or systems for rehabilitation of subjects with neurological disorders, such as the rehabilitation of the locomotor system, including limbs. In one example, the present application discloses an apparatus, more in particular a robotic platform capable of optimizing gravity-dependent trunk movements, enabling overground locomotion in non-ambulatory individuals with spinal cord injury and stroke, while promoting durable motor improvement when delivered during gait rehabilitation facilitated by electrical spinal cord stimulation.

### BACKGROUND AND SUMMARY

Gait recovery after neurological disorders requires re-mastering the interplay between body mechanics and gravitational forces. Despite the importance of gravity-dependent gait interactions and active participation for promoting this learning, these essential components of gait rehabilitation have comparatively received little attention.

Terrestrial locomotion is inherently contingent on the gravitational field created by the mass of the Earth (1). While gravity challenges equilibrium at each step, the gravitational forces acting upon body mechanics enable the generation of ground reaction forces that propel the body forward (2, 3). The bipedal posture of humans exacerbates the impact of gravity on gait and balance (3-6). Due to the elevated position of the center of mass, the human body vaults up and over the stiff stance leg analogous to an inverted pendulum (6), while the contralateral leg performs a near-ballistic oscillation (7). This pendulum mechanism minimizes energy expenditure (2, 6, 8). During early stance, kinetic energy is transformed into potential energy, which is partially recovered as the body falls forward and downward during the second half of stance (2, 3, 5).

The apparent simplicity of the inverted pendulum-like behavior dissimulates sophisticated neurological control mechanisms (9, 10), which require several years of neural development to become mature (11). However, the locomotor impairments resulting from neurological insults such as spinal cord injury (SCI) and stroke stress out the instability of the human gait and the complexity of its neural control.

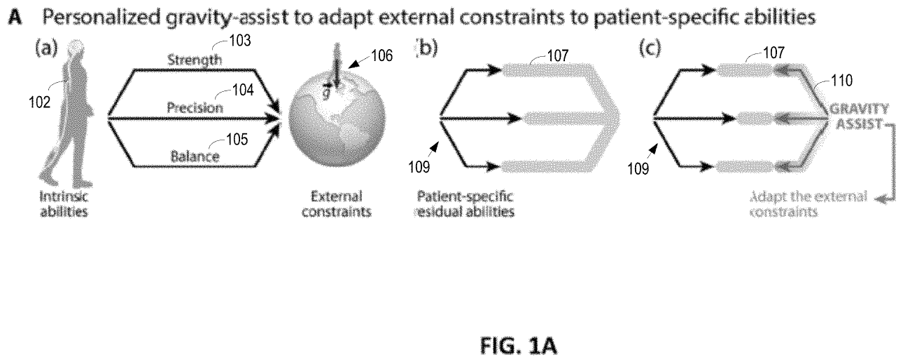

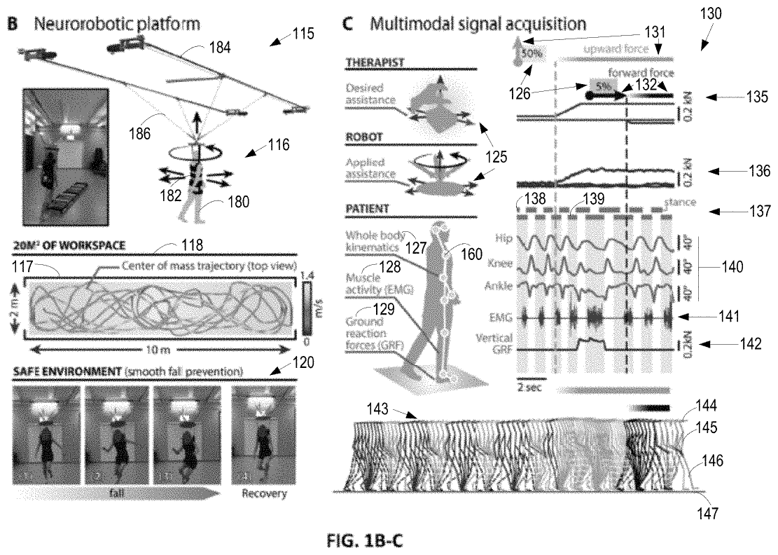

Neurologically impaired individuals must exploit residual neural circuits to regain strength, precision and balance in order to re-master the delicate interplay between body mechanics and gravitational forces (FIG. 1A). Partial bodyweight supported gait therapy is the most common medical practice to facilitate and enhance this process (12, 13). For this, robotic engineering has developed various bodyweight support systems that adapt external constraints to compensate for reduced intrinsic motor abilities. Typically, these systems integrate passive springs, counterweight mechanisms or force-controlled apparatus that deliver trunk support against the direction of gravity during stepping on a treadmill. These approaches suffer from several drawbacks. First, continuous treadmill belt motion dictates the pace of locomotor movements, imposing challenging conditions for neurologically impaired individuals who exhibit variable gait patterns (14). Second, treadmill-restricted environments markedly differ from the rich repertoire of natural locomotor activities underlying daily living. Task-specific rehabilitation is essential to maximize gait recovery (15, 16). Third, vertically restricted trunk support creates undesired anteroposterior and lateral forces that impede gait execution (17, 18). Yet, the recovery of inverted pendulum-like gait movements requires finely tuned trunk movements in multiple directions (4, 19).

The inventors previously developed a robotic postural neuroprosthesis for users that effectively addresses these issues (20). The robotic platform provides versatile trunk support along four degrees of freedom while users are progressing freely within a large workspace. This postural neuroprosthesis enabled skilled locomotion in rodents with moderate SCI and stroke (20-22). The robot also encouraged active participation during gait rehabilitation, which played a pivotal role in promoting activity-dependent neuroplasticity and motor recovery in combination with epidural electrical stimulation of the lumbar spinal cord in subjects with severe SCI (21, 23).

To establish a similar gait rehabilitation platform for humans, the inventors developed (24) a robotic system that allows fine adjustment of forces applied to the trunk along three degrees of freedom. Testing robotic systems in quadrupedal animal model poses further problems. The quadrupedal posture of rats minimizes the mechanical impact of trunk support on dynamic balance. In contrast, the application of forces to the trunk in the bipedal posture of humans is likely to exert additional and specific constraints. Indeed, we found that the application of upward forces alters the inverted pendulum-like gait behavior.

Generally speaking, there is need for a further development in order to translate this neuroprosthetic rehabilitation framework to a treatment that augments motor recovery in human individuals with SCI and other neurological disorders.

Moreover, there is a further need to provide a robotic system for optimizing gait rehabilitation, taking into account the peculiar problems of the bipedal posture of humans.

Individuals with motor disorders require a personalized amount of upward force to compensate for their specific impairments. Currently, therapists select this support from empirical observations. A robotic system capable of adapting its working conditions according to the single user's or user's specific needs is highly desired.

The overall goal is to support the user such that the user learns the dynamics that are involved in walking and/or other activities (e.g. sit-to-stand or stand-to-sit, etc.). This goal can be achieved by providing the user with dynamics that are similar to those of normal walking or other activities, although support is provided.

When only providing a constant vertical unloading force, which is the conventional procedure, the dynamics of the walking task change, for example the gait/stride frequency reduces with unloading. Then, human learning is suboptimal. Therefore, the support forces in all directions have to be adjusted to restore the normal walking dynamics. To restore these dynamics, it is unwanted to impose a certain motion on the user. Instead, the forces on the user should represent the task, while the user can choose his own walking trajectory.

Exemplary measures for a normal walking gait are:
The walking/stride frequency.
The amplitudes of the center of mass motion.
The forward velocity, including fluctuations throughout a gait cycle.
The phase shift between the vertical and horizontal oscillations of the center of mass motion.

Possible further objectives can be more specific amplitudes, phases, and patterns of the legs or of the trunk, for example trunk rotations.

### SUMMARY OF THE APPLICATION

The present application discloses a robotic platform that assists trunk movements in order to optimize gravity-dependent gait interactions during highly participative locomotion or other related tasks within a large and safe environment. An algorithm automatically configures one- or multidirectional forces applied to the trunk based on user-specific needs (patient-specific needs).

Experiments of the inventors showed that this gravity-assist enabled natural walking in non-ambulatory individuals with spinal cord injury and stroke, and allowed less impaired individuals to execute skilled locomotion that they could not perform without robotic assistance. Surprisingly, a single overground training session with gravity-assist improved locomotor performance, whereas walking the same distance on a treadmill did not ameliorate gait. The present application is also effective in gait rehabilitation program in a non-ambulatory individual with a chronic spinal cord injury. To enable motor control during training, we applied epidural electrical stimulation over the lumbar spinal cord using a surgically placed electrode array. This intervention restored walking without robotic assistance and without stimulation. These results highlight the critical importance of precise trunk assistance and active participation to augment motor recovery in response to gait rehabilitation after neurological disorders, and establish a practical pathway to apply these concepts in clinical routine.

In the foregoing, the following definitions are used. The velocities in x, y, and z direction are positive when the person moves forward (x), laterally to the left (y) and upward (z) in its own right-handed body frame (which rotates with the person).

For some embodiments, the user is modeled as a point mass m.

Newton's second law states: [d²x/dt²; d²y/dt²; d²z/dt²]=[Fx; Fy; Fz]/m=F/m, whereby d²x/dt², d²y/dt² and d²z/dt² are the accelerations in antero-posterior, medio-lateral and vertical direction, respectively, and Fx, Fy, Fz are the components of the net force vector F acting on the person.

We define the amount of unloading of the user as Δm (so this is the part of the mass of the user that is compensated by the vertical force).

The sum of the forces on the user is equal to the gravitational force F_{g} plus the force of the person Fₚ (mainly generated by the legs) plus the support force Fₛᵤₚ of the robotic device:

F=F_{g}+Fₚ+Fₛᵤₚ

A conventional body weight support only partially compensates for gravity:

Fₛᵤₚ=[Fₓₛᵤₚ;F_{ysup};F_{zsup}]=[0;0;Δm·g]

This adjusts one aspect of walking and therefore the horizontal forces and the variation in the vertical force become out of proportion to the vertical force the user has to deliver. Or in other words: the weight is compensated, but the inertia of the mass m is still there. The problem of hiding inertia of a robotic device is discussed in H. Vallery; Duschau-Wicke, A. & Riener, R. Hiding Robot Inertia Using Resonance Proceedings of the International Conference of the IEEE Engineering in Medicine and Biology Society (EMBC), 2010, 1271-1274. However, the problem of compensating the effect of inertia of the body mass of the user is still unsolved in the state of the art and a solution is provided by the present application.

In certain aspects of the present application, the user is a subject in need of restoring voluntary control of locomotion, in particular said subject is suffering from neurological disorders and neuromotor impairment.

It is an object of the present application an apparatus comprising a support system for a user, in particular a human user, said apparatus comprising actuators and/or a controller for said support system, said apparatus and/or actuators and/or controller comprising:
a. means for applying one or more of z-direction force F_{zsup}, x-direction force Fₓₛᵤₚ and y-direction force F_{ysup}, or any combination thereof on said user according to the following respective equations:

F_{zsup}=Fz(x,dx/dt,y,dy/dt,z,dz/dt) and;

Fₓₛᵤₚ=Fx(x,dx/dt,y,dy/dt,z,dz/dt);

F_{ysup}=Fy(x,dx/dt,y,dy/dt,z,dz/dt);
wherein
   Fₓₛᵤₚ is the force applied in forward direction,
   F_{ysup} is the force applied in lateral direction and
   F_{zsup} is the force applied in upward direction;
   x, y, and z are the forward, lateral, and vertical coordinate positions of the center of mass in a coordinate system that is fixed to the stance foot and rotates with the person, and dx/dt, dy/dt, dz/dt are the derivatives with respect to time.
b. optionally means for applying further forces on said user.

In certain embodiment of the present application, in said apparatus, said means apply said upward force F_{zsup} according to the following equation:

F_{zsup}=c_{z}(z₀−z)+Δm·g,
wherein
c_{z} is the stiffness;
z is the vertical position of the center of mass of said subject and is approximated/defined as z=Az·sin(ω·t), wherein A is the amplitude of said center of mass motion, ω is the walking frequency,
z₀ is the average or nominal walking height;
Δm is the part of the mass of said subject that is compensated by said upward force;
g is gravity acceleration;
Fₓₛᵤₚ and F_{ysup} are nul.
and wherein the stiffness c_{z} is chosen such that said user walks with a frequency of natural walking. In particular, this can be achieved by calculating c_{z} according to the formula c_{z}=ω² Δm.

In certain embodiments of the present application, in said apparatus, said means apply said forward force Fₓₛᵤₚ is calculated according to the following equations:

Fₓₛᵤₚ=cₓₛ·sin(a_{z}·dz/dt) for z≤z₀,

Fₓₛᵤₚ=0 for z>z₀
wherein
a_{z} and cₓₛ are positive constants;
dz/dt is velocity;
z₀ is the average or nominal walking height;
or according to the following equation:

Fₓₛᵤₚ=Fₓₛᵤₚ(x)

or particularly

Fₓₛᵤₚ=−cₓ·x;
Wherein
c_x is a positive constant
x is the x-position of the center of mass with respect to the stance foot x-position
or according to the following equation:

Fₓₛᵤₚ=Fₓₛᵤₚ(z,dz/dt)

According to the present application, these two latter equations can be used independently or alternating, depending on some other state, depending on the working conditions of the apparatus and on how the user is exploiting it. Such a state can be verified by the skilled person. The support in x-direction may be adjusted constantly to wherever the person steps.

In certain embodiment of the present application, in said apparatus, said means apply said lateral force F_{ysup} according to the following equation:

F_{ysup}=c_{y}·y,
wherein c_{y} is positive or negative stiffness.

In certain embodiment of the present application, in said apparatus, said controller is passive. This is achieved, for example, applying said upward force according to the following equation:

F_{zsup}=F_{zsup}(Fₓₛᵤₚ,dx/dt,dz/dt);
whereby F_{zsup} suffices the following inequality constraint:

F_{zsup}<−Fₓₛᵤₚ(dx/dt)/(dz/dt);

or said forward force such that the maximum energy that can be inserted is lower or equal to a specified virtual energy reservoir, which would particularly be possible using the following equation:

$${F}_{xsup} = \begin{matrix}\{ {F}_{xsup} ( z , dz / dt ) & for dz / dt < 0 and z < {z}_{0}\end{matrix}$$

$$\begin{matrix}\{ {F}_{xsup} ( x ) & for dz / dt > 0 and z < {z}_{0}\end{matrix}$$

$$\begin{matrix}\{ 0 & for z > {z}_{0}\end{matrix}$$

In certain embodiment of the present application, another way as mentioned above is for another degree of freedom is applying an upward force according to the following equation:

Fₓₛᵤₚ=cₓ sin(a_{z}·dz/dt), wherein z<z₀;

To ensure passivity, the controller needs to keep track of the energy that is inserted and set Fₓₛᵤₚ to zero once a pre-specified virtual energy reservoir is empty.

In certain embodiment of the present application, said apparatus further comprises means for measuring the shift of the mean antero-posterior position of the center of plantar pressure of said user and means for applying forward force to said user in order to compensate said shift.

In certain embodiment of the present application, said apparatus, further comprises:
a. means for setting the apparatus in transparent mode;
b. means for computing parameters from kinematic recordings of locomotor tasks performed by said user to obtain and optionally storing a dataset;
c. means for elaborating said dataset with principal component (PC) analysis.

In certain embodiment of the present application, in said apparatus, said means for measuring the shift of the mean antero-posterior position of the center of plantar pressure of said user and means for applying forward force to said user in order to compensate said shift use an artificial neural network.

In certain embodiment of the present application, said apparatus is provided with a recording platform for real-time acquisition of apparatus-subject interactions.

The apparatus according to the present application, and the relevant method for operating it, thanks to the controller herein disclosed, is applicable in every available apparatus and application. For example, it is very well usable also on a 1D body-weight support system on a treadmill. Thanks to the control of the x, y, z directions, the present application can be well exploited in 3D body-weight supports, wearable exoskeleton, etc.

In certain embodiment of the present application, said apparatus is selected from the group consisting of cable robot, trunk support, exoskeleton, wearable exoskeleton and exosuit.

In certain embodiment of the present application, said apparatus also comprises a device for epidural or subdural electrical stimulation.

In certain embodiment of the present application, said apparatus also comprises means c) for applying further forces on said user, for example the therapist or the use himself.

Another object of the present application is the above apparatus for use in restoring voluntary control in a subject, in particular said subject is suffering from a neuromotor impairment. More in particular, said neuromotor impairment is selected from the group consisting of partial or total paralysis of limbs. For example neuromotor impairment is consequent to a spinal cord injury, an ischemic injury resulting from a stroke, a neurodegenerative disease, Amyotrophic Lateral Sclerosis (ALS) or Multiple Sclerosis.

Another object of the present application is a method for operating an apparatus, in particular for control of locomotion, comprising a support system for a user, in particular a human user, said apparatus comprising a controller for said support system, wherein said user is connected to said apparatus, comprising the following steps:
a. setting the apparatus to apply one or more of z-direction force F_{zsup}, x-direction force Fₓₛᵤₚ and y-direction force F_{ysup}, or any combination thereof on said user according to the following respective equations:

Fₓₛᵤₚ=Fx(x,dx/dt,y,dy/dt,z,dz/dt);

F_{ysup}=Fy(x,dx/dt,y,dy/dt,z,dz/dt);

F_{zsup}=Fz(x,dx/dt,y,dy/dt,z,dz/dt);
wherein all the definitions are provided as above.

In certain embodiment, in said method, said upward force F_{zsup} is applied according to the following equation:

F_{zsup}=c_{z}(z₀−z)+Δm·g,

wherein all the definitions are provided as above.
Fₓₛᵤₚ and F_{ysup} are nul.

In certain embodiment, in said method, said forward force Fₓₛᵤₚ is applied according to the following equation, with positive constant a_{z}:

Fₓₛᵤₚ=cₓ·sin(a_{z}·(dz/dt)) for z<z₀,

Fₓₛᵤₚ=0 for z>z₀

wherein all the definitions are provided as above;

or according to the following equations:

Fₓₛᵤₚ={Fₓₛᵤₚ(z,dz/dt)

{Fₓₛᵤₚ(x)

According to the present application, It can be either of these two equations, or these two alternating depending on some other state, as explained above. The support in x-direction has to be adjusted constantly anyway to wherever the person steps.

In certain embodiment, in said method, said lateral force F_{ysup} is applied according to the following equation:

F_{ysup}=c_{y}·y.

wherein all the definitions are provided as above.

In certain embodiment, said method comprises the following steps:
a. setting the apparatus to apply an upward force on said subject in quiet standing;
b. measuring the shift of the mean antero-posterior position of the center of plantar pressure of said subject of the postural maintenance of said subject;
c. setting the apparatus to apply a forward force to said subject in order to compensate said shift.

In certain embodiment, said method comprises the following steps:
a. setting the apparatus in transparent mode;
b. having said subject to perform locomotor task;
c. computing parameters from kinematic recordings from said locomotor task to obtain a dataset;
d. submitting said dataset to a principal component (PC) analysis to provide a quantification of locomotor performance of said subject, and extracting parameters accounting for the effects of experimental conditions on locomotor performance of said subject;
e. setting the apparatus to apply an upward force on said subject in quiet standing;
f. measuring the shift of the mean antero-posterior position of the center of plantar pressure of said subject] of the postural maintenance of said subject;
g. setting the apparatus to apply a forward force to said subject in order to compensate said shift.

In certain embodiment, said method comprises the following steps:
a. setting the apparatus in transparent mode, with a first or second subject in standing position, wherein said first subject is a normal subject and said second subject is a subject in need of restoring voluntary control of locomotion;
b. recording whole-body kinematics, ground reaction forces and ankle muscle activity over the maximal possible range of upward forces for said first subject to obtain a first dataset;
c. recording whole-body kinematics, ground reaction forces and ankle muscle activity over the maximal possible range of upward forces for said second subject to obtain a second dataset;
d. applying a Principal Component analysis on said first and second dataset and determining the upward force as the condition with the minimum distance between said second subject and said first healthy subject in the Principal Component space;
e. setting the apparatus to apply an upward force on said second subject in quiet standing;
f. measuring the shift of the mean antero-posterior position of the center of plantar pressure of between said first subject and said second subject;
g. setting the apparatus to apply a forward force to said second subject in order to compensate said shift.

In certain embodiment, step d) is performed using an artificial neural network.

In certain embodiment, step g) is performed setting said forward force as a function of walking speed of said second subject.

It is another object of the present application a computer program for carrying out the above method.

It is another object of the present application a data medium having the above a computer program.

It is another object of the present application a computer system on which the above computer program is loaded.

It is another object of the present application the above apparatus operatively connected to said computer system.

Preferably, the above method steps are executed in an automated way, by means of algorithms. For example, applying a Principal Component analysis on said first and second dataset and determining the upward force as the condition with the minimum distance between said subject in need of restoring voluntary control and said healthy subject in the Principal Component space is performed using an artificial neural network.

The present application will be now disclosed in details also by means of Figures and examples.

Please note that any issues or problems or disadvantages and advantages discussed herein are recognized by the inventors and no admissions are made as to these being known in the art.

In the Figures:

FIG. 1: Conceptual and technological framework of the gravity-assist. (A) A combination of strength 103, precision 104 and balance 105 are necessary to walk in the gravitational environment of the Earth 106 (a). Neurological deficits induce a gap 107 between these intrinsic motor control abilities 109 (including strength, precision, and balance) and the external constraints, which prevents independent walking (b). The gravity-assist 110 aims at reducing this gap 107 by adapting the external constraints to patient-specific residual motor control abilities (c). (B) Schematic and photograph of the robotic support system 115, including the directions of the actuated and passive (rotation) degrees of freedom 116. The plot 117 represents the spatial trajectory and instantaneous speed of the center of mass (CoM) during free locomotion of a healthy subject within the entire workspace 118 covered by the robot (e.g. 115). A subject 180 may be coupled to the support system via a harness 182 attached to a plurality of tracks 184 via one or more cables 186. The sequence of photographs 120 (1 to 4) illustrates the recovery from a fall during walking. (C) The amount of force applied to the trunk in the upward, mediolateral and forward directions are adjustable independently. The arrows indicate the direction of the force. The actual forces are measured by sensors embedded in the robot, but are expressed as a percent 126 of the subject's total bodyweight for readability. Whole body kinematics 127, electromyographic (EMG) 128 activity of leg muscles and ground reaction forces (GRF) 129 are recorded concomitantly. In some examples, kinematic activity (e.g. 127) may be recorded via sensors or markers 160 placed strategically on the subject. A gait sequence 130 is shown during which an upward force 131 followed by an increasing forward force 132 are applied to a healthy subject during walking. From top to bottom are shown: desired forces 135, measured forces 136, stance durations 137 of the left 138 and right 139 legs, changes in leg joint angles 140, muscle activity 141, vertical component of the GRFs 142 when stepping onto the force-plate, and the timing of applied forces. The corresponding stick diagram decomposition 143 (rate, 12 ms) of the head 144, trunk 145 and leg 146 movements during the stance (dark) and swing (light) phases of locomotion is shown at the bottom. The filled and dashed lines differentiate the right and left legs, respectively. The grey shading 147 indicates the onset and end of upward and forward forces.

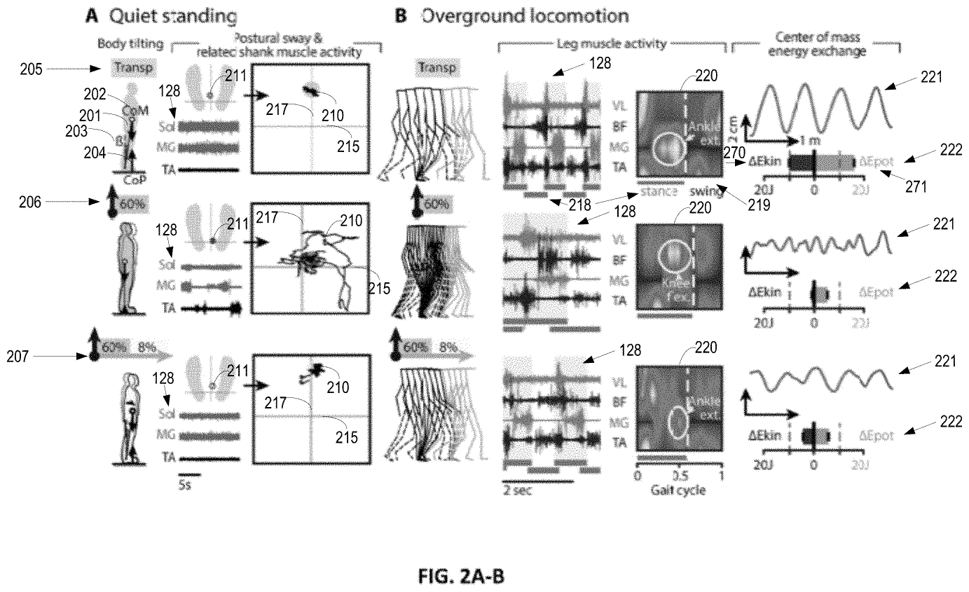

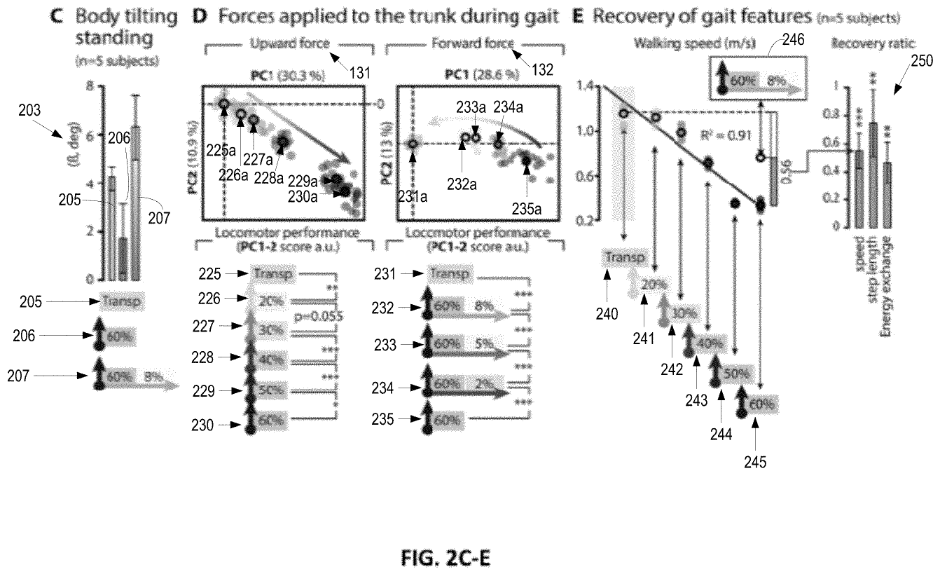

FIG. 2: Interaction between upward and forward forces during standing and walking. (A) Schematic of the body 201, including the CoM 202, the postural orientation (β) 203, and the center of foot pressure (CoP) 204. The mean position of the CoP with respect to the feet is shown during standing with transparent (Transp) support 205, upward force only 206, and both upward and forward forces 207. A concomitant sequence of EMG 128 activity from ankle extensor (soleus, Sol; medial gastrocnemius, MG) and ankle flexor muscles (tibialis anterior, TA) is displayed. The plot represents the continuous 210 and mean 211 (colored circle) positions of the CoP for each condition. The X-axis 215 refers to the axis passing through the malleoli, while the Y-axis 217 corresponds to the midline between the feet. (B) Stick diagram decomposition of whole-body movement using the same conventions as in FIG. 1. The EMG activity 128 of extensor (ext) and flexor (flex) muscles acting at the ankle and knee (vastus lateralis, VL; biceps femoris, BF) is displayed, together with a heat map 220 showing the activity of these muscles averaged over multiple steps (n=10). Stance 218 is represented as a solid line, whereas swing 219 is represented as the absence of the solid line. The trajectory 221 of the CoM in the sagittal plane is shown for each condition. The bar plots 222 indicate the associated variations of kinetic energy (ΔEkin) 270 and gravitational potential energy (ΔEpot) 271. (C) Mean postural orientation 203 during standing under the conditions shown in (A). (D) Gait kinematics of one subject shown in the space created by PC1 and PC2 (%, explained variance). Each color-coded dot corresponds to a single gait cycle, while the circles indicate the average value for each condition. ***p<0.001, **p<0.01, *p<0.05, Mann-Whitney U tests. Conditions for upward force (transparent, 20%, 30%, 40%, 50%, 60%), are labeled 225-230, respectively. The associated circles, comprising the average value for each condition are represented as 225a, 226a, 227a, 228a, 229a, 230a, respectively. Similarly, conditions for forward force (transparent, 60% upward/8% forward, 60% upward/5% forward, 60% upward/2% forward, and 60% upward), are labeled as 231-235, respectively. The associated circles, comprising the average value for each condition, are represented as 231a, 232a, 233a, 234a, and 235a, respectively. (E) Relationships between the upward force and the walking speed in the absence of forward force, and when applying a correction 246, shown for the larger upward force (245). Transparent, 20%, 30%, 40%, 50% and 60% upward force is indicated by numbers 240-245. The relative recovery of basic gait features was calculated as a recovery ratio 250, and represented in the plot for 5 healthy subjects. ***p<0.001, **p<0.01, Paired Student's t-test. Data are means+/−s.e.m. (n indicated in figure).

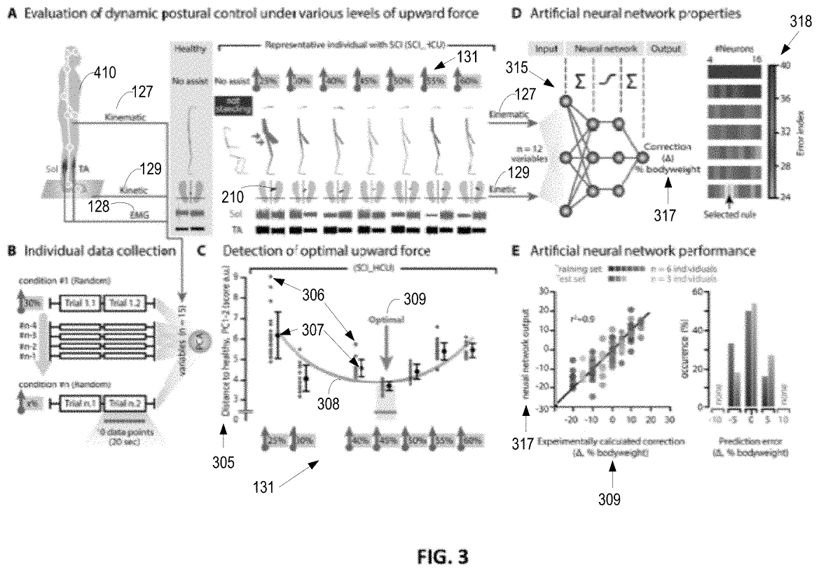

FIG. 3: Algorithm predicting the personalized, optimal upward force of the gravity-assist (A) Representative stick diagram decomposition of whole-body movements/kinematics (e.g. 127), continuous CoP trajectory (e.g. 210), and EMG activity (e.g. 128) of ankle muscles during standing with upward forces (e.g. 131) ranging from 25 to 60% of bodyweight support with 5% increments for a non-ambulatory individual with a SCI. (B) Scheme summarizing collection and processing of individual data on which the PCA was applied. (C) Plot showing the relationship between the upward force (e.g. 131) and the Euclidian distance 305 between the data of the participant 310 shown in (A) and healthy subjects in the space defined by PC1 and PC2. Individual trials 306 and mean values 307 are represented by grey dots and circles with S.D. bars, respectively. A parabolic fitting 308 was applied to the data to highlight the occurrence of a minima, which was labeled as optimal 309. (D) The measured variables (e.g. 127, 129) (n=12) are fed into an artificial neural network 315 that calculates the correction of bodyweight support 317 (A, upward force in % of bodyweight) to facilitate gait execution. The color plots 318 illustrate the iterative process that led to the selection of the number of neurons and learning rules that minimized errors. (E) Relationship between the optimal correction (e.g. 309) of the upward force to facilitate locomotion, which was measured experimentally (PCA), and the corresponding prediction (e.g. 317) of the neural network. Each dot corresponds to a given condition of upward force for a subject with SCI or stroke who contributed to the training or test data set. The histogram plot reports the occurrence rate of errors in the prediction of the corrections calculated by the artificial neural network.

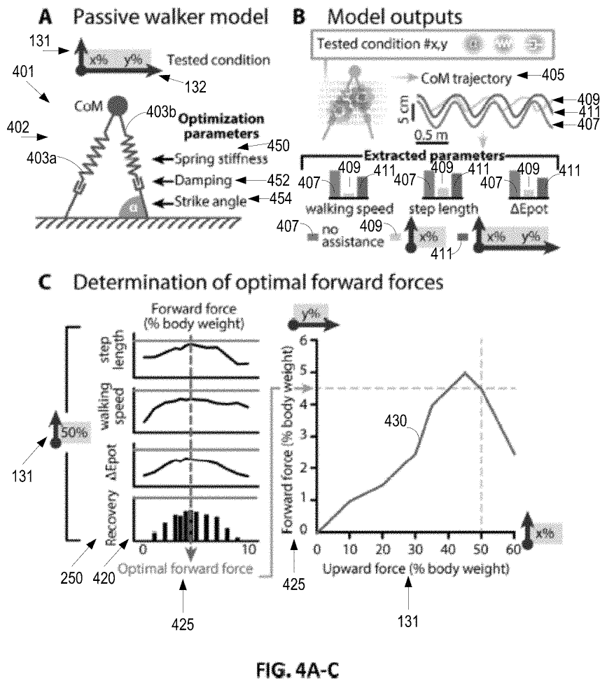

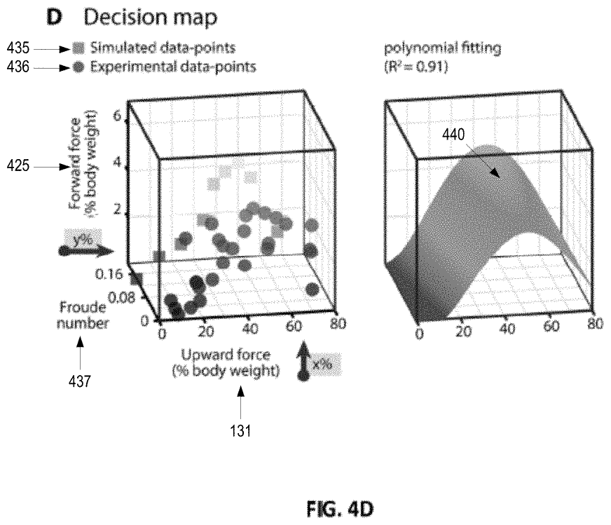

FIG. 4: Decision map to configure the optimal forward force correction for locomotion. (A) Schematic of the passive walker model 401. Briefly, an inverted pendulum-like gait behavior 402 was simulated using the model 401 where gravity is sufficient to promote continuous, alternating oscillations of the limbs 403a, 403b. Consequently, the intrinsic mechanical properties of the model determine the walking speed. Optimization parameters of the model include spring stiffness 450, damping, 452, and strike angle 454. Tested conditions of the model may include upward (e.g. 131) and forward (e.g. 132) forces. As observed in human subjects, the application of upward forces substantially reduced walking speed, step length, and energetic exchanges. (B) Illustration of the output and experiments with the model. The computed trajectory of the CoM 405 is shown without support 407, with upward force 409 (light grey), and with both upward and forward forces 411 (dark grey). The bar plots show the walking speed, step length, and variation in gravitational potential energy (ΔEpot) for each condition. (C) Changes in the step length, walking speed and variation in gravitational potential energy (ΔEpot) over the entire range of forward force while applying an upward force (e.g. 131) corresponding to 50% of the bodyweight. The histogram plot 420 reports the recovery ratio (e.g. 250) associated with each forward force, which was calculated from the 3 parameters (step length, walking speed, (ΔEpot)) depicted above. The highest recovery ratio was extracted, labelled as the optimal forward force 425, and reported in a plot 430 indicating the optimal forward force (e.g. 425) for each upward force (e.g. 131) at the speed determined by the mechanical properties of the model. (D) 3D plots reporting the relationships between the upward force (e.g. 131), the optimal forward force (e.g. 425), and the walking speed. Each data point corresponds to values measured in the simulations 435 and in subjects with SCI or stroke 436. To include data from subjects with varying biometric properties, the speed is represented as the Froude number 437, which takes into account the length of the pendulum to normalize the walking speed. A polynomial function 440 was fitted through the simulated (e.g. 435) and experimental (e.g. 436) data points.

FIG. 5: Accuracy of the algorithm to configure the gravity-assist for individuals with SCI or stroke. (A) Stick diagram decomposition (e.g. 143), CoP trajectory (e.g. 210) and muscle activity (e.g. 128) recorded in a subject 571 with SCI during standing with 3 levels of upward force (e.g. 131). More specifically, the three levels of force are indicated as 550a (30% upward and 3% forward, 550b (40% upward and 4% forward), and 550c (50% upward and 5% forward) The artificial neural network (e.g. 315) calculated, for each upward force condition independently, the necessary correction of the upward force (e.g. optimal upward force 309) to facilitate gait execution for this specific subject. (B) Stick diagram decomposition (e.g. 143) of whole body movements and of the walker during locomotion. A representative sequence of EMG activity (e.g. 128) recorded from ankle muscles is reported for each condition, together with the stance duration (e.g. 218) of each leg (grey bar). Foot trajectory 505 is illustrated for each condition. Upward force (e.g. 131) and forward force (e.g. 132) is illustrated for each condition. (C) Spatial trajectory of the CoM (e.g. 405) under each condition of upward (e.g. 131) and forward (e.g. 132) forces, including the associated ΔEkin (e.g. 270) and ΔEpot (e.g. 271) that reflects gait efficacy. (D) A PCA was applied on the kinematic (e.g. 127) variables measured from all the gait cycles from the subject shown in (A-C) and in healthy individuals (n=8). Conventions are the same as in FIG. 2. More specifically, the three force levels (550a-c) are indicated. Also indicated is health individual (E) Plot reporting the locomotor performance of individual subjects with SCI or stroke with the gravity-assist (GA) and with plus (550c) and minus (550a) 10% of upward force (e.g. 131). Locomotor performance was measured as the Euclidian distance (e.g. 305) between gait cycles of the subject 560 and of healthy individuals 565 in the space defined by PC1 and PC2. ***p<0.001, Repeated measures 2-ways ANOVA. Data are means+/−s.e.m. (n indicated in figure).

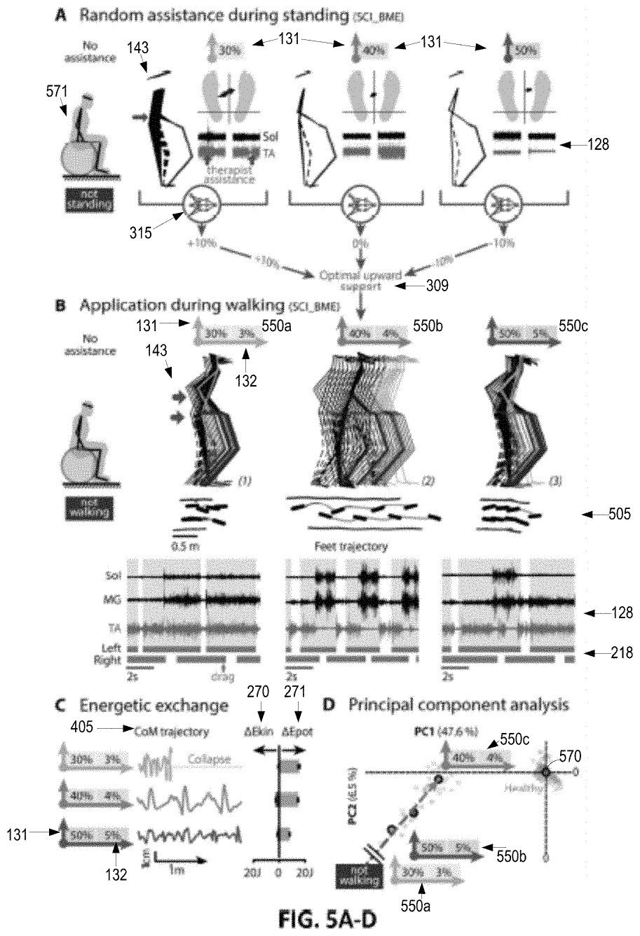

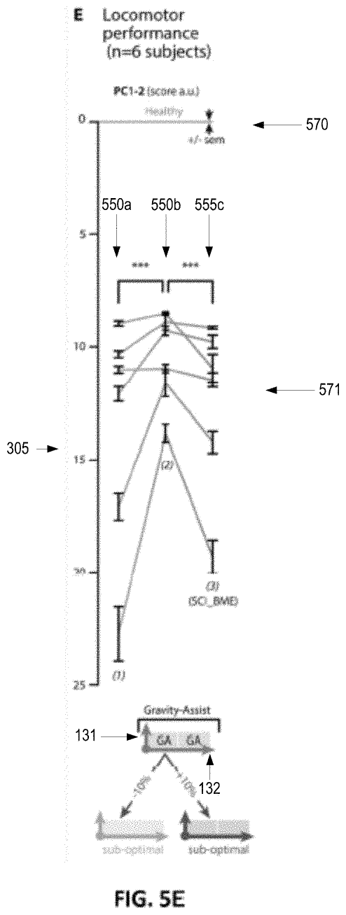

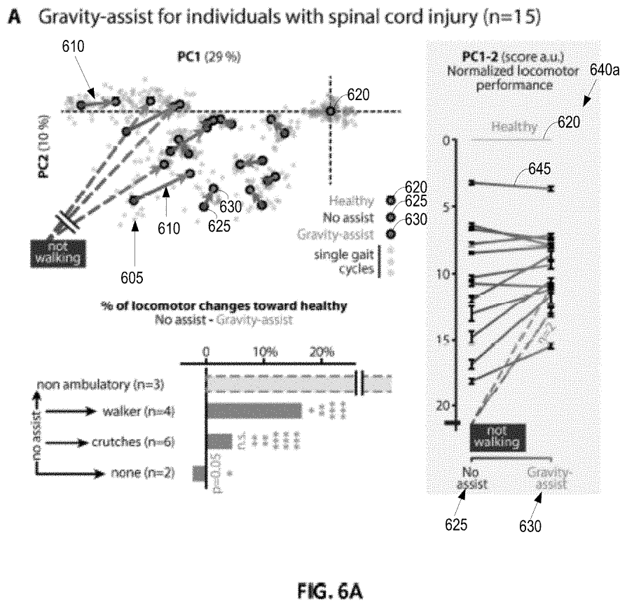

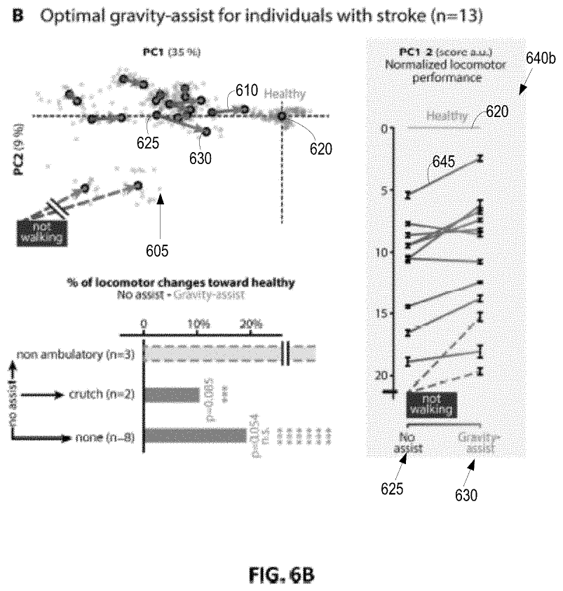

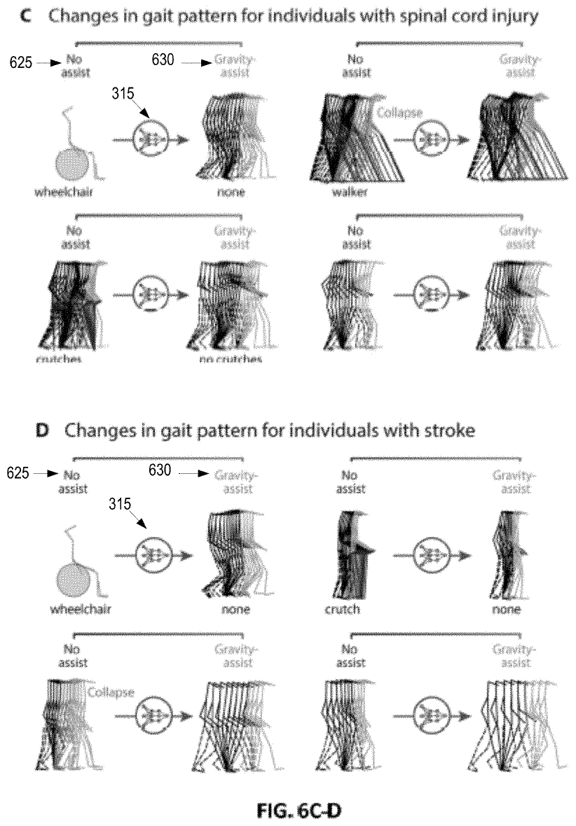

FIG. 6: Performance of the gravity-assist to facilitate locomotion after SCI and stroke. Two cohorts of individuals with (A) various severities of SCI, and (B) various severities of stroke were recorded during locomotion without robotic assistance and with the gravity-assist. The subjects were allowed to use their preferred assistive device if necessary, which segregated them into four categories: non-ambulatory, crutches, walker, none. A PCA was applied on all the kinematic variables (e.g. 127) measured from all the gait cycles of all the subjects with SCI or stroke and on healthy individuals. Single gait cycles 605, together with the average (circles with black diameter) per condition, are reported in the space created by PC1 and PC2. Conventions are the same as in FIG. 5. For each subject, the arrow 610 indicates the shift in the average location of gait cycles in the PC space during locomotion with gravity-assist. Average values for healthy individuals 620, individuals with no assist 625, and with gravity assist 630, are illustrated. While only one shift is labeled with the no assist 625 and gravity assist 630, it may be understood that in each case shown, the arrow 610 points to the gravity assist 630 as shown. This shift in locomotor performance is quantified in the nearby plot 640a for FIG. 6A and 640b for FIG. 6B showing, for each subject, the relative position of gait cycles in both conditions (no assist 625 and gravity assist 630) with respect to gait cycles of healthy individuals 620. The horizontal bars 645? report these improvements in percent of change across subjects for each category. The statistical differences between both conditions is reported for each subject independently. ***p<0.001, **p<0.01, *p<0.05, n.s. non-significant, Mann Whitney U-test. Representative stick diagram decomposition of whole body movements (and assistive device) during locomotion without robotic assistance and with gravity-assist for a representative subject in each category of (C) SCI and (D) stroke severities. For (C) and (D), no assist 625 is illustrated once for clarity, gravity assist 630 is illustrated once for clarity, and the neural network (e.g. 315) is illustrated for clarity. Data are means+/−s.e.m. (n indicated in figure).

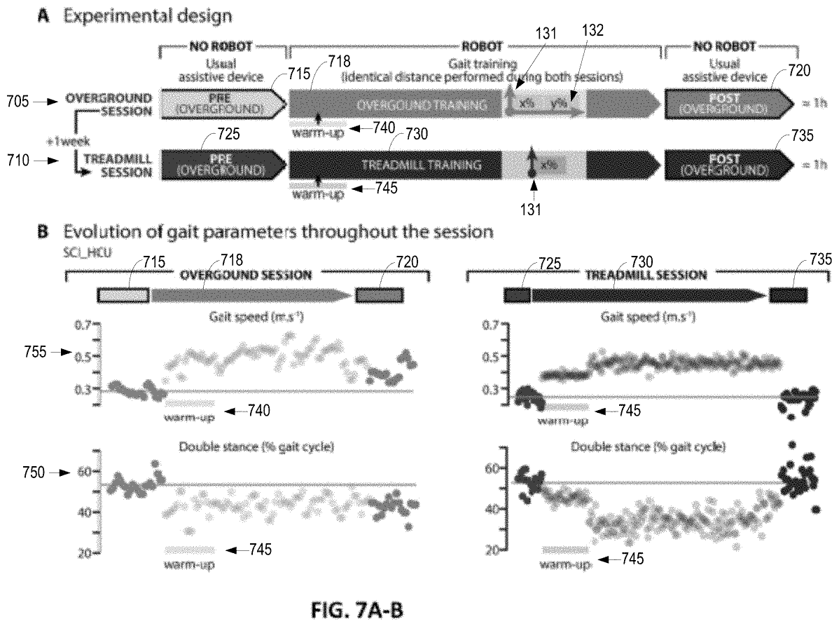

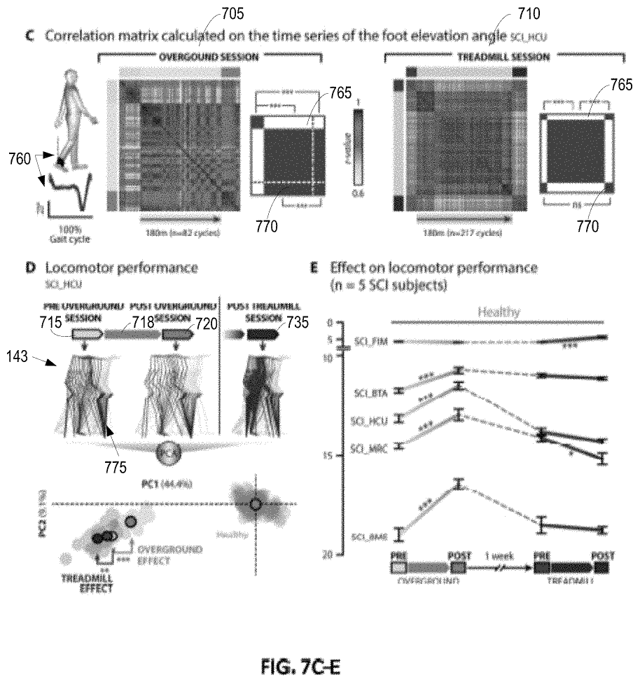

FIG. 7: Gait rehabilitation overground with gravity-assist vs. on a treadmill with upward support. (A) Experimental design. The experimental design includes an overground session 705, and a treadmill session 710. Overground session 705 includes a pre-overground session 715 in the absence of robotic assistance, followed by overground training 718 with robotic assistance (e.g. upward force 131 and forward force 132). Subsequent to the overground training 718, a post overground session 720 is conducted in the absence of robotic assistance. Treadmill session 710 includes a pre-overground treadmill session 725, followed by treadmill training 730 with robotic assistance (e.g. upward force 131). Subsequent to the treadmill training 730, a post treadmill overground training 735 is conducted in the absence of robotic assistance. Overground session 705 includes overground warm-up session 740, and treadmill session 710 includes treadmill warm-up session 745. (B) Stick diagram decompositions of whole body movements for subject SCI_HCU before and after each training session. (B) Plots reporting the double stance duration 750 and gait speed 755 for each successive gait cycle of subject SCI_HCU over the course of the entire session. The color coding refers to the experimental design detailed in (A). (C) Correlation matrix for the foot elevation angle 760 with respect to the direction of gravity (inset) for each gait cycle. The recording time windows are indicated using the color coding detailed in (A). The white 765 and grey 770 regions in the nearby rectangles indicate statistical differences between gait cycles recorded during the contrasted time windows. Kruskal Wallis with Tukey-Kramer post hoc tests. (D) Representative stick diagram (e.g. 143) decomposition of whole-body movements including a crutch 775 recorded overground without robotic assistance. Locomotor performance was evaluated using the PCA based method described in FIG. 5. Mann-Whitney U tests. (E) Plot reporting the locomotor performance of individual subjects before and after training with gravity-assist overground, as well as one week later before and after training restricted to a treadmill. Mann-Whitney U tests. Conventions are the same as in FIG. 5. ***p<0.001, **p<0.01, *p<0.05, n.s. non significant. Data are means+/−s.e.m. (n indicated in figure).

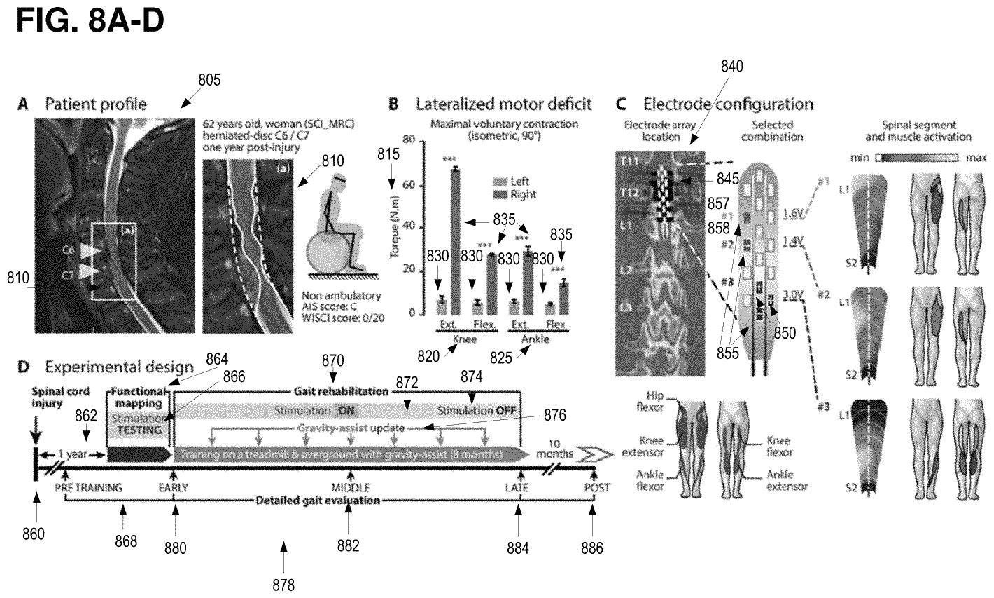

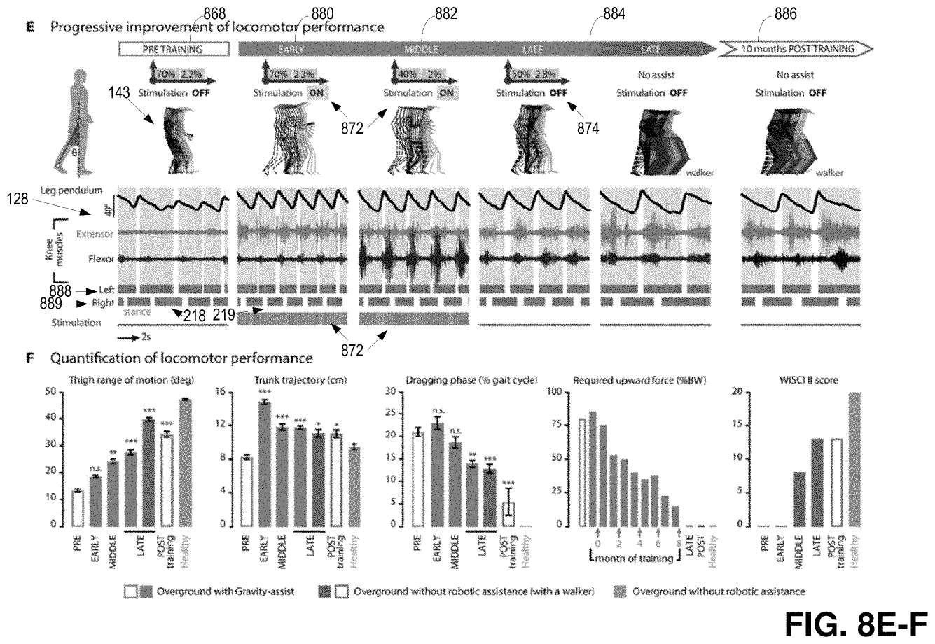

FIG. 8: Gait rehabilitation with gravity-assist enabled by electrical spinal cord stimulation. (A) Clinical profile of the participant. Magnetic Resonance Imaging (MRI) of the cervical spinal cord 805, including a zoom 810 on the lesioned region. (B) Maximal isometric torque 815 towards extension and flexion produced at the knee 820 and ankle 825 of the left 830 and right 835 legs while in a sitting position with a 90 deg angle at each joint. (C) MRI 840 of the surgically implanted epidural array 845, including a scheme of the cathode 850 (+) and anode 855 (−) configurations for each stimulation site. For site #1 857 and #2 858, the case of the stimulator served as the anode. For each stimulation site, the activation of spinal segments was calculated by projecting the normalized amplitude of motor responses measured in leg muscles acting at each joint of the leg onto the known location of motoneurons in the lumbosacral spinal cord. The relative activation of leg muscles is displayed in the color-coded scheme. (D) Experimental design of the gait rehabilitation program. The experimental design includes a duration 862 (e.g. 1 year) between a spinal cord injury 860 prior to functional mapping 864 and stimulation testing 866. Such functional mapping and stimulation testing may take place in a pre-training phase 868. Following the pre-training phase, a gait rehabilitation phase 870 with stimulation ON 872 for a majority of the gait rehabilitation phase, followed by stimulation OFF 874, is conducted. The gait rehabilitation phase may be understood to comprise training on a treadmill and overground with gravity assist for a period of approximately 3 months, however the period of time may be shorter or longer in some examples. During the gait rehabilitation phase 870 there may be a periodic gravity assist update 876, where target gravity-assist forces (e.g. upward force, lateral force, forward force, etc.) are reexamined. Detailed gait evaluations 878 may be made during the pre-training phase 868, and during early 880, middle 882, late, 884, and post training 886 phases of the experimental manipulation. The post training phase 886 may comprise roughly 10 months in some examples, but may be greater than or less than 10 months in other examples. (E) Stick diagram decomposition (e.g. 1423) of whole body movement together with the oscillation of the leg (e.g. stance 218 and swing 219) in the sagittal plane for left 888 and right 889 legs and EMG activity (e.g. 128) of knee muscles during locomotion recorded throughout the gait rehabilitation program under the experimental conditions indicated above each panel. Conventions are the same as in FIG. 5. As illustrated above in (D), simulation is OFF during the pretraining phase, and late phase of the gait rehabilitation program. Simulation is ON during the early and middle phases. Furthermore, during the late phase 884, simulation is first turned off while the gravity assist (e.g. forward and upward forces) is continued 874. Subsequently, both the gravity assist and stimulation are turned off. (F) Bar plots reporting the mean values of a subset of gait features and WISCI II score measured at the same time points and under the same conditions as those shown in the panels. The right bar plot indicates the evolution of the upward force necessary to enable the participant to walk overground. ***p<0.001, **p<0.01, *p<0.05 compared to pre-training condition, Kruskal Wallis with Tukey Kramer post hoc tests. Data are means+/−s.e.m.

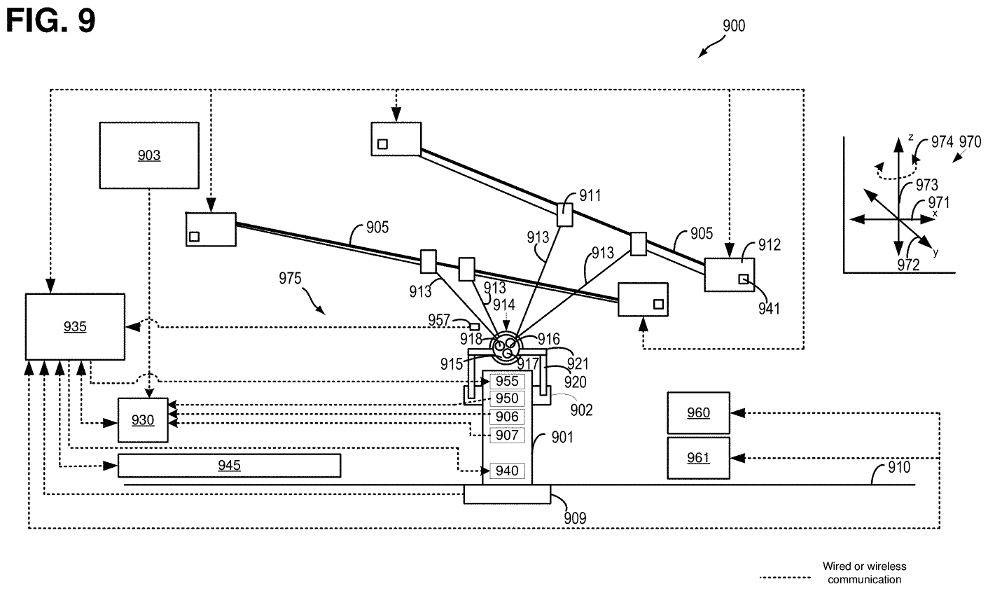

FIG. 9: Detailed description of robotic support system 900 (e.g. 115). Robotic support system 900 may include one or more rails 905 (e.g. 184). In one example, said one or more rails are attached to a ceiling (not shown) in a horizontal fashion, and which may be tilted (e.g. 45 degrees) towards a workspace 975 (e.g. 118). Each rail 905 guides two deflection units 911 composed of a ball-beared cart carrying an inclinable pulley (not shown). The inclination axis of the pulley is parallel to the rail. A cable (e.g. Dyneema) 913 (e.g. 186) connects the two carts on one rail in order to form trolleys. Motorized winches (e.g. actuators) 912 actuating the cables may be positioned at the extremities of the rails. It may be understood that the cables, when not under tension, can only exert force in one direction (e.g. the cable may “pull” but not “push”). Four elastic elements (not shown) consisting of spiral steel springs each with a parallel rubber cord inside connect the cables to stainless steel rings. The arrangement allows the cables to intersect at a specific point, termed node 914 (a similar system is e.g. disclosed in WO 2017/005661 A1). In other words, the cables may connect to one point, which may thus result in the absence of a moment applied to the subject which may otherwise occur if the cables connected to the subject at two or more positions. Winch positions may be measured by encoders 941 on the motor shafts, while a length of the elastic elements (not shown) is monitored using wire potentiometers 957. Wire potentiometers 957 are shown as a box in close proximity to the cables, as the four elastic elements are not shown due to space restriction. An inertial measurement unit (IMU) 915 combining accelerometers 916, gyroscopes 917, and a magnetometer 918 are located in the node. These sensors provide redundant information allowing to calculate the position of the node and resultant force vector(s) on the subject through optimization. Communication procedures may be implemented in Matlab using an EtherCat network operating at 1 kHz (57). Commands may be sent from a controller 935, to the motorized winches, to control a plurality of forces applied to the subject. While a cable system is illustrated, it may be understood that in some examples, connections to the subject to provide desired forces acting on the subject may be rigid, unlike cables which may be at least partly elastomeric in nature.

A subject 901 (e.g. 180) may be attached to the robot using a harness 902 (e.g. 182). Two shoulder straps 920 of the harness are attached to two outer ends of a plate 921 by means of buckles (not shown). The plate itself is pivot-mounted to the lower end of the node. The plate can rotate infinitely, allowing the subject to take arbitrary turns. The robot may thus enable subjects to walk freely within the workspace. In some examples, the workspace may comprise 20 m² (10 m length by 2 m width by 2.6 m height). However, in other examples, the workspace may be larger, or smaller than 20 m². The robot may support 100 kg or more, and may have a maximal vertical (z, see inset 970) support of 90 kg and a maximal forward force of +/−5 kg in the lateral (y) and longitudinal (x) directions. A fall detector and smooth counteraction mechanism may guarantee patient safety in case of a fall.

Robotic support system 900 may include a physiological recording unit 930. Physiological recording unit may monitor kinematics, kinetics, and muscle activity signals, for example. More specifically, bipolar surface electrodes 907 (e.g. 1 cm diameter, electrode separation of 1 cm) may be placed over one or more leg muscles to record electromyographic activity (e.g. 128). Leg muscles for recording electromyographic activity may include soleus, medial and lateral gastrocnemius, tibialis anterior, semitendinosus, biceps femoris, vastus lateralis and rectus femoris, for example. Electromyographic activity may be monitored via a 16-channel wireless recording system (Myon 320, Myon AG, Switzerland), in some examples. Kinematic recordings (e.g. 127) may be obtained using a real-time 3D motion capture system 903. In some examples, motion capture system may comprise fourteen Bonita10 cameras and two Bonita720c DV cameras (Vicon, UK). Trunk, head and bilateral leg and arm kinematics may be recorded using a plurality of markers 906 (e.g. 160) positioned overlying anatomical landmarks. In some examples, anatomical landmarks may be defined by a full-body kinematic model (e.g. Plug-In-Gait, Vicon). In some examples, movement of assistive devices (e.g. crutches, walker, cane, etc.) may additionally be monitored using position markers 906 (e.g. 160). Position markers 906 may be of a reflective type, meaning for example that they reflect infrared light emitted by cameras comprising the motion capture system 903, thus allowing the tracking of limb movements by the subject. However, position markers may be of other types as well. Examples of other position markers 906 may include optical systems, electromagnetic systems, ultrasonic systems, and combinations of systems suitably integrated by what is known as the “sensor fusion” method, including a triangulation system using radio frequency antennae and an inertial sensor. The location or position of position markers 906 may be acquired in real-time and may be associated with specific labels (for example crest, hip, knee, ankle, foot) according to the kinematic model (e.g. user-defined). It may be understood that “real-time” in this example refers to the controller continuously receiving input from the markers while the subject is performing a task. Such input may be received via the controller with minimal delay (e.g. less than 1 second) from when it was captured via the motion capture system 903. In some examples, the kinematic model may evaluate a set of rules which may compare X, Y, and Z coordinates of each marker, and may derive which set of coordinates corresponds to which label (e.g. crest, hip, knee, ankle, foot). The body of subject 901 may thus be modeled as an interconnected chain of rigid segments. Anthropometric data (body height, body weight, widths of the joints) may additionally be added to the model to determine the positions of joint centers, and calculate the elevation and joint angles of the lower limbs. A ground reaction vector and antero-posterior and medio-lateral torques (e.g. kinetic activity 129) may be acquired using force plates 909 integrated in the floor 910.

In some examples, epidural and/or subdural electrical stimulation may be applied to the subject while the subject is performing a training routine which may include a gravity-assisted training routine, or other training routine (e.g. treadmill 945 with or without gravity assist, for example). For example, epidural and/or subdural electrical stimulation may be provided via a means for electrical stimulation 940 (e.g. 845), also referred to herein as a means for neuromodulation, with adjustable stimulation parameters. As an example, the means for electrical stimulation 940 may include one or more electrodes, an electrode array, an implantable pulse generator, etc. The means for electrical stimulation 940 may be utilized to apply epidural and/or subdural electrical stimulation to any stimulation site along the spinal cord of the subject. For example, stimulation sites may be lumbar and sacral sites for lower limb stimulation and may be cervical sites for optional upper-limb stimulation. Lower limb stimulation may be applied, for example, for facilitating standing and walking in a subject, while upper-limb stimulation may be applied, for example for facilitating reaching and grasping. In one example of the disclosure, said stimulation sites may be one, and may be turned on and off depending on specific sub-phases of gait (e.g. phase dependent). In another example of the disclosure, stimulation sites may be at least two, where each stimulation site may be independently turned on or off depending on specific sub-phases of gait. Thus, it may be understood that the means for electrical stimulation 940 may be activated to promote whole-limb flexion or extension, for example. In some examples, said means for electrical stimulation 940 may provide a burst stimulation. In such an example, it may be understood that each electrode (or each multi-electrode array) may be activated for a certain time (“burst”), wherein the activation times of each electrode (or multi-electrode array), and the duration of each activation is pre-defined by a user (not shown), said user being a clinician or physiotherapist, for example. In an example, electrical stimulation provided via the means for electrical stimulation 940 may be location-specific, wherein stimulation parameters (e.g. waveform, amplitude, pulse width, frequency) of each individual electrode may be modified in real-time. In yet another example, electrical stimulation provided via the means for electrical stimulation 940 may be time-specific, where each single electrode may be individually turned ON and OFF in real time based on external trigger signals (e.g. in relation to monitored kinematic, kinetic, and/or EMG activity). In still another example, electrical stimulation provided via the means for electrical stimulation may be frequency-specific. For example the means for electrical stimulation 940 may provide a stimulation frequency comprised of between 5 and 120 Hz, more specifically between 25 and 95 Hz, wherein the resolution is, for example, of 1 Hz.

In another example, the controller may acquire neural signals from the subject via neural sensors 950. Said neural signals may provide information about the locomotor state and the neuronal activity of the subject to physiological recording unit 930, and which may then be transmitted to signal processing device or controller 935. As an example, neural signals may provide information related to the gait cycle of the subject, and may be used to control or refine in real time (e.g. minimal delay, such as less than 5 seconds or less than 1 second) the triggering of the means for electrical stimulation 940, respectively substituting or co-operating with the kinematic-feedback algorithms described above.

In an example, said neural sensors may include electrode arrays implanted in the limb area of the sensorimotor cortex of the subject, and may collect information about a locomotor intention of the subject. Using machine-learning approaches, for example, such information may be decoded and discriminated into two behavioral states, “rest” or “walk”. The decoded information may then be transmitted to signal processing device or controller 935, which may switch ON or OFF the means for electrical stimulation, such that a desired locomotor pattern may be achieved.

In some examples, the controller may allow deriving, at each gait-cycle, an optimal electrical stimulation and/or epidural electrical stimulation frequency on the basis of a desired locomotion feature output. The feature output may be entered by the user, based on a desired behavior, for example. The controller may then tune automatically the electrical stimulation to make sure an observed behavior matches the reference output. More specifically, a program stored at the controller may comprise a feedforward component, and a feedback component. Said feedforward component may comprise an input-output linear model, which may allow to directly derive the most suitable electrical stimulation frequency given the desired reference output at each gait-cycle, and to minimize control delays. Said reference output may comprise a locomotion feature, also termed gait feature herein. Said gait feature may include step height, for example, a maximum height reached by a foot of the subject during each gait cycle.

Feedforward component may thus capture observed relationships between stimulation and gait features. Such information may then be utilized to predict and automatically tune stimulation so as to modulate output behavior.

Feedforward component may be constantly updated using adaptive fitting algorithms known in the art (e.g. Least Mean Squares or other methods for linear or non-linear regression) which may take into account fatigue and time-varying characteristics of the locomotor system of the subject.

The feedback component (e.g. a Proportional Integral Control part) of the controller may compensate for modeling errors or unexpected disturbances. At each gait-cycle, the feedback component may calculate an “error” value between a measured output and desired, or reference, output. On the basis of the calculated error value, the feedback component may adjust the input so as to minimize said error.

In yet another example, a means for pharmacological stimulation 955 (e.g. a device for pharmacological stimulation) may be included. For example, pharmacological stimulation, or treatment, may be provided to the subject via means known to those in the art. For example, means for pharmacological stimulation 955 may include injection via a needle, where said injection is controllable via the controller. However, other means for pharmaceutical stimulation may include substances taken orally, applied to the skin (e.g. topical), transmucosal application, inhalation, etc. In one example, the system of the disclosure may be used in combination with a pharmacological treatment via the means for pharmacological stimulation 955, for further facilitating locomotor functions. In particular a pharmaceutical composition comprising at least one agonist to monoaminergic receptors, for example serotonergic, dopaminergic, and adrenergic receptors, may be administered to the subject.

Inset 970 illustrates directions of force which may be applied to the subject. The actuated forces which may be applied to the subject via the robotic support structure include x (horizontal) 971, y (lateral) 972, and z (vertical) 973 are illustrated as solid lines, while passive (rotation) forces 974 that the subject may undergo within the constraints of the workspace is illustrated as a dashed line.

Returning to the robotic support structure 900, dashed lines stemming from or feeding into the controller 935 illustrate wired or wireless communication between the referenced items discussed herein.

Furthermore, there may in some examples be augmented reality items 960 which may be controllable via the controller. More specifically, one example may include a series of shapes aligned on a floor space of the workspace, which may light up in desired sequence patterns, such that a subject may engage in a type of game involving movement-related exercises. In some examples, feedback from a subject conducting a particular training routine may be used to modify an augmented reality program for the particular routine. For example, based on one or more of kinematic, kinetic, electromyographic and/or force data (from the IMU) recorded from the subjects movements, optimal placement of particular augmented reality items may be determined. Optimal placement may include placement where it is likely that a subject may be capable of reaching, for example. More specifically, the augmented reality may comprise shapes which may be projected on a floor of the workspace. Based on the above-mentioned recorded variables from the subject, a path established via the shapes may change such that it is likely the subject will be able to accomplish the task without adverse or undesirable movements. Other examples of augmented reality are entirely within the scope of this disclosure. In some examples, virtual reality 961 may similarly be utilized in conjunction with a particular training routine or routines. By incorporating virtual reality 961 and/or augmented reality 960 into training procedures, the repetitive nature of rehabilitative training routines may be made more enjoyable to the subject conducting the procedures.

### DETAILED DESCRIPTION OF THE APPLICATION

According to the present application, the term “user” is referred to a subject using the above apparatus. The term “user” and “subject” are interchangeable. In particular, the user is a human.

According to certain embodiments, the “user” is a “patient” in need of regaining locomotion control, as better specified in the foregoing.

All the following embodiments can be formulated within the general equations:

Fₓₛᵤₚ=Fx(x,dx/dt,y,dy/dt,z,dz/dt)

F_{ysup}=Fy(x,dx/dt,y,dy/dt,z,dz/dt)

F_{zsup}=Fz(x,dx/dt,y,dy/dt,z,dz/dt),

Such that all force components are functions of the movement of the user in all directions and possibly of other variables.

### Controlling the Z-Direction

The vertical position z of the center of mass (CoM) of the user is approximately a sinusoidal function of time with amplitude Az and frequency ω:

z=A_{z}·sin(ω·t)

In one embodiment, this can be exploited to support both the weight as the inertia of the user in vertical direction. This leads to a spring-like behavior of the force:

F_{zsup}=c_{z}·(z₀−z)+Δm·g

Where z₀ is the average or nominal walking height (conveniently obtained calibration of the therapist, or by low-pass filtering of z) and the stiffness c_{z} depends on the mass that has to be unloaded. In general, Fz can be made a suitable linear or non-linear function of z:

F_{zsup}=F_{zsup}(z)

In one embodiment, the robot takes care of the “extra inertia” that is left over when gravity is taken away by choosing c_{z} such that ω=sqrt(c_{z}/Δm), so that means c_{z}=ω²·Δm. That means the inertia of Δm will oscillate in synchrony with the human.

In an alternative embodiment, c_{z} is tuned by the therapist or by an adaptive algorithm until the gait frequency has achieved a desired value (for example a physiological value).

Controlling the x-Direction (Forward or Antero-Posterior)

In normal human walking, the motion of the person in x-direction can be split into two phases.

When z>z₀(z₀ is the average walking height, so around apex), the acceleration d²x is approximately zero. When z<z₀, the acceleration approximates a sinusoidal function of the velocity dz/dt in z-direction, with positive constants hₓ and a_{z}:

d²x/dt²=hₓ·sin(a_{z}·dz/dt);

wherein hₓ is a constant.

One embodiment to restore natural dynamics is that the force in x-direction is made a function of z and dz/dt:

Fₓₛᵤₚ=cₓ·sin(a_{z}·(dz/dt)) for z≤z₀

Fₓₛᵤₚ=0 for z>z₀,

With conveniently chosen cₓ, for example by manual tuning, or by inverse identification from human motion data. Hereby, the primed force components are added to the previously chosen values for Fₓₛᵤₚ, which are needed to ensure vertical oscillations

Or in general:

Fₓₛᵤₚ=Fₓₛᵤₚ(x,dx/dt,z,dz/dt)

Another embodiment is to make Fₓₛᵤₚ a function of x. Due to the accelerations of the person, this leads to a negative virtual spring stiffness:

Fₓₛᵤₚ=−cₓ·x

Where cₓ is negative. Or in general: Fₓₛᵤₚ=Fₓₛᵤₚ(x)

A third embodiment is to make a combination of the two above, which proves to be beneficial in the section on passivity described below:

Fₓₛᵤₚ={Fₓₛᵤₚ(z,dz/dt)

{Fₓₛᵤₚ(x)

Controlling the y-Direction

In y-direction, a similar approach can be taken as for the x-direction. Here, again a suitable y₀ is defined, which relates to the average lateral position of the person (for example obtained by low-pass filtering or set by the user). Then, the force in y-direction is conveniently chosen as a function of this y-position, using a positive or negative stiffness c_{y}:

F_{ysup}=c_{y}·y

This compensates for the excess inertia in y-direction compared to the moment that gravity can generate about the support points.

The design can be similar as for z or x-direction, for example to ensure the correct gait frequency or relative phase between the different oscillations (It needs to be taken into account that the oscillation in y-direction has half the frequency of the oscillations in x- and z-directions).

### Passivity

For stability of the combined system user/user-robotic apparatus, it is very beneficial if the controller is passive. According to the present application, at least two methods are able to ensure this.

A first option is to extract energy in the vertical direction when inserting energy in the horizontal direction. To ensure this at every instant in time, the following relationship on powers should hold:

F_{zsup}·dz/dt<−Fₓₛᵤₚ·dx/dt,

With velocities dx/dt and dz/dt in x and z-direction. respectively.

Or rewritten:

F_{zsup}<−Fx·(dₓₛᵤₚ/dt)/(dz/dt)

In general, we can define any function:

F_{zsup}=F_{zsup}(Fₓₛᵤₚ,dx/dt,dz/dt)

that suffices the inequality constraint above, and this guarantees passivity.

Another option is to split the part where |Fₓₛᵤₚ|>0 in two parts: one in which we extract energy and one in which we insert energy. In the control for the x-direction above, one of the options is:

Fₓₛᵤₚ=cx·sin(a_{z}·dz/dt) for z<z₀

Now during one cycle (which starts with heel strike of one leg and ends with heel strike of the same leg), the velocity dx is typically positive (so directed upwards), dz starts negative (so directed downwards) and ends positive. This means that it is started with a period of energy extraction and the cycle is ended with a period of energy insertion. To guarantee passivity, it has to be ensured that a less or equal amount of energy is inserted than extracted. This can be seen as a reservoir of energy that is filled during the first phase and emptied during the second phase. Two methods to do this are disclosed as follows:

a) Keep track of the energy that is inserted and set Fₓₛᵤₚ to zero once the energy reservoir is empty.

b) Calculate a force field (Fₓₛᵤₚ=Fₓₛᵤₚ(x)) such that the maximum insertible energy is lower or equal to the energy reservoir.

In the second option, the control law becomes:

$${F}_{xsup} = \begin{matrix}\{ {F}_{xsup} ( z , dz / dt ) & for dz / dt < 0 \& z < {z}_{0}\end{matrix}$$

$$\begin{matrix}\{ {F}_{xsup} ( x ) & for dz / dt > 0 \& z < {z}_{0}\end{matrix}$$

$$\begin{matrix}\{ 0 & for z > {z}_{0}\end{matrix}$$

All the functions described above can be optimized for the purpose of achieving normal walking as described under ‘goal’. For this optimization, we disclose four methods:

a) Obtain the functions based on human data;

b) Obtain the functions based on literature, for example based on Zijlstra W, H of AL. Displacement of the pelvis during human walking: experimental data and model predictions. Gait Posture 1997; 6:249-262;

c) Make an adaptive controller that optimizes parameterized functions;

d) Let a human user (such as the therapist or the user) tune the parameters.

The apparatus disclosed in the present application can be managed in many ways, according to the general knowledge in this field. For example, a control system as disclosed in WO2015063127 can be adopted.

In the following, a detailed description of one possible embodiment of the present application is provided, applying a forward force (x direction), being understood that the also the lateral force (y direction), optionally together the forward force, can be applied according to the same principle.

Previously, a cable robot (24) was developed that provides a safe environment preventing falls, while allowing high-precision control of forces applied to the trunk along the three Cartesian directions during unconstrained locomotion in a large workspace (FIG. 1B). A robotic interface was integrated within a recording platform (26) allowing real-time acquisition of robotic movement, forces applied to the trunk, whole-body kinematics, ground reaction forces and muscle activity (FIG. 1C). A real-time Ethernet network enabled the platform to control robot-subject interactions and augmented reality scenarios in real-time based on any recorded modalities.

To verify the transparency of the robot, whole-body kinematics were compared during walking without and with robot in eight healthy individuals. The robot was configured in transparent mode, which corresponds to the minimal upward force (4 kg) necessary to enable robot-subject interactions. A large number of conventional parameters was computed from kinematic recordings (n=120), and submitted this dataset to a principal component (PC) analysis. This method allows the precise quantification of locomotor performance, and the objective extraction of parameters accounting for the effects of experimental conditions on locomotor performance (20, 27). Despite this comprehensive evaluation, no differences were detected between gait patterns without and with robot (P=0.69, Paired Student's t-test). These results were confirmed by the inventors during walking along a sinusoidal path (n=6 subjects) Even in such challenging conditions, the robot did not alter the acceleration profile of the center of mass (CoM) and gait patterns, indicating that the robotic attachment did not interfere with the production of gait.

### Impact of Upward and Forward Forces on Posture and Gait

Next, the biomechanical and functional consequences of upward and forward forces were evaluated on whole-body kinematics and leg muscle activity during standing and walking in healthy individuals.

Firstly, the impact of upward forces during quiet standing was studied. During standing and walking, the human body can be modeled as an inverted pendulum (FIG. 2A). Due to the natural forward tilt of the body (β), the CoM projects in front of the rotational axis (ankle) of the pendulum. Consequently, the mean antero-posterior position of the center of plantar pressure (CoP) is located at 25+/−1% of the base of support length (n=5 subjects). This biomechanical configuration allows the maintenance of balance through the tonic activation of ankle extensor muscles (FIG. 2A).

The application of upward forces with the robot (40 to 500N) induced a backward tilt of the body pendulum (β, FIG. 2C), which led to a shift in the CoP position towards the heels. Under these conditions, subjects displayed increased postural sways, paralleled by considerable changes in ankle muscle activity patterns that transited from tonic activation of extensor muscles to alternating activity between extensor and flexor muscles (FIG. 2A).

It was found that the application of an additional forward force (0 to 50N) was able to compensate for the detrimental effects of upward forces on postural maintenance. Forward forces restored the natural position and dynamics of the CoP, which reestablished appropriate patterns of ankle muscle activity (FIG. 2A).

It was also found that the occurrence of similar interactions between upward and forward forces during locomotion. The application of upward forces alone led to a gradual alteration of gait features (FIG. 2B,D; P<0.05, Mann-Whitney U-test). Negative correlations between the amount of upward force and the alteration of key gait features were detected such as the step length (R²=0.78) and walking speed (R²=0.91; FIG. 2E). During locomotion, inverted pendulum-like gait movements propel the body forward with minimal effort. At a high-level of upward force, the forward progression required an abnormal activation of knee flexor muscles during late stance in order to pull the body forward (FIG. 2B).

As observed during standing, the addition of forward forces alleviated the alteration of whole-body kinematics and muscle activity (FIG. 2B). The gradual improvement of gait patterns with increasing forward forces (FIG. 2D; P<0.001, Mann-Whitney U-test) mediated a significant recovery of the natural step length and walking speed (FIG. 2E; P<0.01, Paired Student's t-tests). Forward forces also improved the exchanges between kinematic energy and potential energy associated with inverted pendulum-like gait movements (FIG. 2B,E; P<0.01, Paired Student's t-tests).

These results highlight critical interactions between upward and forward forces on the production of posture and gait with robotic assistance, stressing the importance of developing evidence-based procedures to configure these forces based on user-specific needs.

### Design of Gravity-Assist Algorithm: Personalization of Upward Force

The robotic system herein disclosed allows configuring the upward force for each subject based on objective measurements.

The inventors enrolled nine subjects with SCI or stroke that were not able to stand without assistive devices. To determine the optimal upward force during standing, we recorded whole-body kinematics, ground reaction forces and ankle muscle activity over the maximal possible range of upward forces for each subject (from 15% to 70% of bodyweight, 49%+/−14%, mean+/−s.d.; FIG. 3A,B). Then, a PC analysis on all the computed parameters (n=15) recorded in each subject and healthy individuals (n=5) was applied. The optimal amount of upward force was defined as the condition with the minimum distance between the tested subject and healthy individuals in the PC space (FIG. 3C).

While this method is objective, it requires an extensive recording session that cannot be performed in daily clinical practice. The inventors thus aimed at developing an algorithm to automatize this procedure. For this, a supervised machine learning was implemented using an artificial neural network that predicted the optimal amount of upward force for each subject based on a subset of easily and rapidly collected kinematic and kinetic variables (n=12, muscle activity was not included due to the preparation time and idiosyncratic characteristics of signals). The artificial neural network was fed with a training set (64 trials, n=6 subjects) restricted to 20 s of recording at various levels of upward forces (FIG. 3D). The number of neurons and rules was selected that minimized errors in the predicted corrections of upward forces (A, % of bodyweight). Testing on independent datasets (32 trials, n=3 subjects) revealed the ability of the artificial neural network to calculate corrections with an error that never exceeded 5% of the experimentally measured optimal upward force (FIG. 3E).

Then it was sought to develop an algorithm that automatically calibrates the forward force based on the selected upward force and user-specific needs.

An inverted pendulum-like gait behavior was simulated using a passive walker model wherein gravity is sufficient to promote continuous, alternating oscillations of the limbs (FIG. 4A). Consequently, the intrinsic mechanical properties of the model determined the walking speed. As observed in human subjects, the application of upward forces substantially reduced the walking speed, the step length, and the energetic exchanges.

Simulations were performed to identify the optimal forward force for each upward force applied to the passive walker (FIG. 4B). Simulations searched the optimal configuration to normalize the walking speed, the step length and gravity-dependent energetic exchanges towards values obtained without any external force. An inverted, U-shape curve was obtained defining the optimal configuration of forces for a given walking speed (FIG. 4C).

Individuals with neurological deficits exhibit a preferred walking speed at which their locomotor performance is optimal. In the model, the optimal forward force is directly related to the walking speed. Therefore, we sought to create a map integrating the preferred walking speed in the configuration of the gravity-assist. For this, we recorded locomotion in a cohort of subjects with SCI or stroke (n=28 subjects) using the upward force calculated by the artificial neural network, and a narrow range of forward forces centered around the optimal values predicted by the simulations. We applied a PC analysis on the collected kinematic parameters to identify the forward force that maximized locomotor performance. To elaborate a decision map that configured the upward and forward forces for each subject, we fitted an optimal polynomial function to the experimental and simulated data points (FIG. 4D). We used this map to personalize the gravity-assist in all the subsequent evaluations and training sessions.

Validation of the Gravity-Assist to Enable Locomotion in Individuals with SCI or Stroke

The inventors next evaluated the accuracy of the gravity-assist algorithm to establish optimal upward and forward forces in order to enable locomotion in individuals with neurological deficits.

The gravity-assist algorithm was tested in 6 individuals with an SCI or a stroke. The subjects were first recorded during standing with the optimal upward force, and with the addition or subtraction of an upward force corresponding to 10% of their bodyweight. For each condition of upward forces, kinematic and kinetic recordings were independently submitted to the artificial neural network, which calculated upward force corrections to establish the optimal condition for each subject. For example, FIG. 5A illustrates kinematic and kinetic recordings for one of the subjects included in the testing dataset of the artificial neural network. This subject was not capable of standing or walking independently (SCI-BME, AIS-C). The artificial neural network yielded the same predictions, regardless of the initial upward force. We used this correction and the preferred walking speed to configure the forward force using the decision map (FIG. 4D).

The personalized gravity-assist enabled this subject to progress overground with coordinated, weight-bearing locomotor movements (FIG. 5B). Improved dynamics of the CoM and associated energetic exchanges revealed the partial restoration of an inverted pendulum-like gait behavior (FIG. 5C). A 10% increase or decrease in the amount of upward force drastically altered gait features, almost preventing this subject to progress forward (FIG. 5B).

To measure the accuracy of the gravity-assist algorithm in each subject, a PC analysis was applied on all the kinematic parameters (n=120, FIG. 5D). This analysis showed that the gravity-assist algorithm yielded optimal configurations in all the tested subjects (FIG. 5E; P<0.001, repeated measures two-ways ANOVA). The beneficial impact of the gravity-assist on locomotor performance increased with the severity of gait deficits.

These results illustrate the sensibility of the gravity-assist configuration to enable locomotion in neurologically impaired subjects, and validate the capacity of the algorithm to optimally configure the gravity-assist based on user-specific needs.

The Gravity-Assist Improves Locomotor Performance in Individuals with SCI and Stroke

Next the capacity of the gravity-assist was tested to improve locomotor performance in two cohorts of individuals with varying severities of SCI (n=15) and stroke (n=13). Locomotor performances ranged from non-ambulatory individuals who could not stand nor walk independently, to individuals with mild motor impairments who could walk without an assistive device.

With the exception of non-ambulatory individuals, all the subjects were first recorded during locomotion without the robot using the assistive device that they used in their daily life. All the subjects were then evaluated during locomotion with gravity-assist, both with and without assistive device when possible. To quantify locomotor performance, the ensemble of kinematic variables (n=120) recorded in healthy individuals (n=13) and in all the subjects with SCI (FIG. 6A) or stroke (FIG. 6B) was submitted to separate PC analyses. Locomotor performance was quantified as the distance between injured subjects and healthy individuals in the space defined by PC1 and PC2.

This analysis revealed that the gravity-assist enabled walking in non-ambulatory subjects with locomotor performance similar to ambulatory subjects, both after SCI (FIG. 6A) and stroke (FIG. 6B). Each subject showed specific responses for the gait analyses without assistance and with gravity-assist for all the 28 individuals with SCI or stroke.

To identify the specific characteristics improved by the gravity-assist, the parameters were extracted that highly correlated with PC1 and PC2 (factor loadings, |value|>0.5) and regrouped them into functional clusters corresponding to basic gait features. First this analysis was cinducted for individuals with SCI. It was found that PC1 quantified improvements of leg kinematics, while PC2 captured changes in postural control. The improvements depended on the initial locomotor performance (R²=0.64, P<0.005), as illustrated for representative subjects in FIG. 6C. For example, the subjects who could not stand independently were capable of walking overground with or without assistive device (3/3 subjects, FIG. 6C) while using the gravity-assist. Subjects who were only able to locomote with crutches or a walker progressed without assistive device (4/10 subjects, FIG. 6C) with the gravity-assist, and exhibited improvements in spatiotemporal gait features. Subjects who were able to walk without assistive device did not exhibit systematic changes in leg kinematics (data points closer to healthy individuals) with the gravity-assist. In these subjects, however, the gravity-assist increased postural stability (P<0.05, Kruskal-Wallis with Tukey-Kramer post hoc test). Moreover, the gravity-assist enabled these subjects to execute locomotor tasks that they considered too challenging or too risky to perform in their daily life. For example, these subjects were capable of climbing up a staircase and progressing with accurate foot placement along the rungs of a horizontal ladder placed 20 cm above the ground.

Individuals with stroke exhibited similar or even superior amelioration of locomotor performance with the gravity-assist (FIG. 6B). Extraction of functional clusters from the PC analysis revealed that the initial locomotor performance determined the specific gait features improved by the gravity-assist. For example, individuals who only walked with crutches regained the ability to adduct their paretic foot, which normalized the foot position during stance (P<0.001, Kruskal-Wallis with Tukey-Kramer post hoc test). Individuals who walked without assistive device showed a distinctly enhanced control over distal joints (P<0.001, Kruskal-Wallis with Tukey-Kramer post hoc test). In general, all the individuals with stroke exhibited improved postural control, which was evident in the reduction of lateral trunk sways and increased coordination between upper and lower limb oscillations. As observed in subjects with SCI, the gravity-assist enabled individuals with a stroke to execute skilled locomotion that they could not perform without robotic assistance.

Together, these results show that the gravity-assist enabled or improved locomotor performance in individuals with severe to moderate gait deficits due to SCI or stroke. The gravity-assist allowed less affected individuals to perform activities of daily living that require skilled and finely balanced movements, thus providing the unique opportunity to train natural locomotor behaviors in neurologically impaired individuals.

The Gravity-Assist Improved Locomotor Performance after One Gait Training Session

Then it was sought to demonstrate the superior ability of the overground walking environment with gravity-assist to improve locomotor performance compared to standard treadmill-restricted stepping conditions.

Five subjects were enrolled with chronic SCI who were capable of walking overground, but only with an assistive device. They participated in two training sessions, separated by one week (FIG. 7A). During the first session (60 min), subjects walked overground with gravity-assist. During the second training session (week 2), they were asked to walk the same distance on a treadmill with the same upward force, but without forward force corrections. Immediately before and after each training session, the subjects were recorded during overground locomotion without gravity-assist at their own selected pace using their preferred assistive device.

With the exception of the least affected subject, the training session with gravity-assist mediated a significant increase in overground locomotor performance in all the participants (FIG. 7E; P<0.001, Mann-Whitney U-test). For example, the increase in walking speed and decrease in double stance duration enabled by the gravity-assist throughout training persisted during overground locomotion without gravity-assist (FIG. 7B; P<0.001, Mann-Whitney U-test). However, evaluations conducted one week later revealed that these improvements had vanished (FIG. 7B,D,E).

In contrast, overground locomotor performance remained unchanged or even deteriorated after treadmill-restricted step training (FIG. 7B,D,E). Basic gait features such as walking speed and double stance duration improved during training, but the values returned to baseline levels when the subjects walked overground with their assistive device after training (FIG. 7B,E).

To study locomotor improvements throughout both training sessions, the changes in the elevation angle of the foot were analyzed with respect to the direction of gravity (FIG. 7C). This gait feature plays a critical role in the production of inverted pendulum-like gait movements in humans (28). A correlation matrix was computed on the time series of foot elevation angles extracted from every performed gait cycles, including pre- and post-training recordings. Whereas foot elevation angles remained unchanged before and after treadmill-restricted step training, improved control over foot movements enabled by the gravity-assist during gait training persisted during post-training evaluations (FIG. 7C; P<0.001, Kruskal Wallis with Tukey-Kramer post hoc tests).

An interview with questionnaire indicated an overwhelming preference of participants for overground gait training with gravity-assist compared to the treadmill-restricted step training.

These results show that a one-hour gait training session enabled by the gravity-assist mediated significant improvements of overground locomotor performance immediately after training. In contrast, walking the same distance in a treadmill-restricted environment tended to deteriorate locomotor performance.

Training with Gravity-Assist and Electrical Spinal Cord Stimulation Promotes Durable Recovery

A woman was enrolled who had suffered a cervical SCI. Her locomotor recovery had plateaued after a conventional rehabilitation program that took placed 4 times per week during 12 months after the injury (62 yo, AIS-C, FIG. 8A). She had previously been implanted with an epidural electrode array over lumbar spinal cord segments to alleviate neuropathic pain in the legs (FIG. 8C). This implant was used to conduct a preliminary study evaluating the long-term impact of gait rehabilitation with gravity-assist and electrical spinal cord stimulation.

To evaluate residual supraspinal access to the leg musculature, the maximum voluntary torque production of the left and right legs in a sitting position was recorded (FIG. 8B). The participant exhibited some control over the muscles innervating the right leg, but had nearly no access to muscles innervating the left leg. The severe deficits of the left leg considerably limited the duration and distance over which the participant was capable of walking with gravity-assist during gait training. To augment locomotor performance, the configurations of epidural electrodes was searched that effectively targeted proprioceptive feedback circuits engaging muscles of the left leg (29, 30). For this, single pulses of monopolar electrical stimulation (pulse width: 0.15 ms, intensity: 1 to 3 mA, frequency 2 Hz) were delivered through each electrode of the array in a lying position.

Simultaneous electromyographic recordings of proprioceptive reflex responses evoked in multiple muscles of the left and right legs allowed the reconstruction of spinal segment activation for each site of stimulation. Based on this mapping, the electrode configurations were selected that maximized the activation of proprioceptive feedback circuits embedded in the left hemicord of lumbar segments (FIG. 8C).

Continuous (40 Hz) stimulation through these electrodes enabled the participant to access otherwise quiescent muscles of the left leg during locomotion with gravity-assist (FIG. 8E; P<0.05, Mann-Whitney U-test). The resulting improvements of locomotor performance included a significant increase in walking speed, stride length, step height, and amplitude of leg movements (P<0.001, Mann-Whitney U-tests).

After completion of these evaluations, the participant followed a gait rehabilitation program combining treadmill-based training and overground locomotion with gravity-assist, as previously details in rodents (23). The stimulation protocol was delivered throughout the one-hour training session that took place 2 to 3 times per week (FIG. 8D). Over time, improved locomotor performance allowed the reduction of upward force. After three months of rehabilitation, the participant had regained a volitional control of leg muscles that she could not access before training (FIG. 8E). To encourage the recovery of natural walking, we gradually decreased and eventually withdrew the stimulation during training. The gravity-assist allowed a progressive transition into overground locomotion with a walker and at home. At the end of the gait rehabilitation program, the participant was capable of walking overground using a walker without robotic assistance and without stimulation. Her WISCI-II score had thus increased from zero to thirteen, while her AIS score converted from C to D.

Evaluations conducted ten months after completion of the gait rehabilitation program revealed that the improvement of AIS score and locomotor performance had remained stable (FIG. 8E).

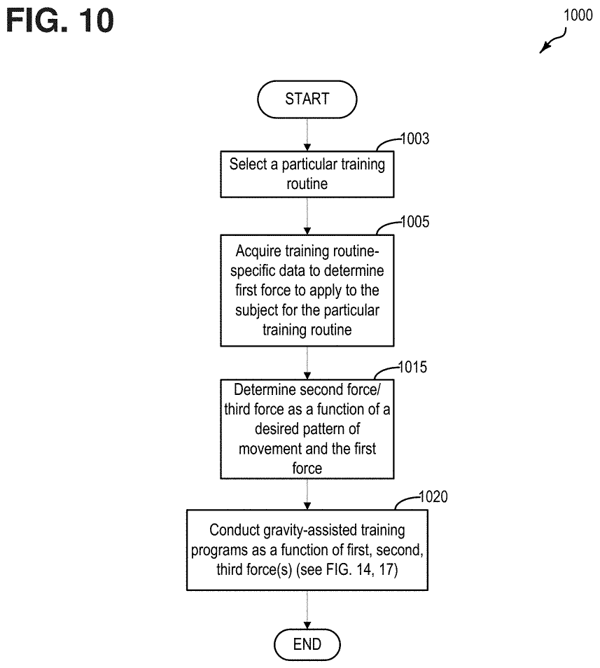

Turning now to FIG. 10, a high-level example method 1000 is shown for selecting a particular training routine for a subject, and acquiring a set of forces to apply to the subject to assist the subject in performing the particular training routine. In examples, depending on the particular training routine selected (or sub-phase of a routine, see below), said set of forces to apply to the subject may be different. Such a set of forces may include one or more of a first force, second force, and third force, for example. Such first force, second force, and third force may be controlled/regulated via a robotic support system (e.g. 900), and applied via one or more cables attaching to a common attachment point on the patient, or to different points on the patient.

Method 1000 may be carried out at least in part, via a controller (e.g. 935), associated with the robotic support system. The selected set of forces may be fixed forces independent of patient movement and/or time-varying forces, and or combinations thereof.

Method 1000 begins at 1003 and may include selecting a particular training routine from a set of available training routines for the subject. In one example, the training routine may comprise a sitting-to-standing routine. In another example, the training routine may comprise a standing-to-sitting routine. Another example may comprise a routine where the subject walks overground. In some examples, the training routine may comprise walking on a treadmill. Another example may include a routine where the subject attempts to jog or run, either overground or a treadmill. Still another example may include a routine where the subject cycles on a stationary bicycle. Yet another example may include a routine where the subject progresses overground on a bicycle. Other examples may include a routine where the subject progresses overground in a sigmoidal curve, where the subject goes from a standing or sitting down position to a lying down position and vice versa, where the subject progresses up or down stairs, where the subject walks on a regularly or irregularly-spaced ladder, etc. In one example, two or more, and/or all of the above examples, may be organized in a set of available routines from which a selection is made such that the selected routine is then performed for the particular training/rehab session of interest. Such examples are meant to be illustrative, and are not meant to be limiting.

As mentioned, particular forces applied to the subject may vary between the different training routines. For example, a set of forces applied to the subject for an overground walking routine may be different than a set of forces applied to the subject for a sitting-to-standing procedure. The magnitude of forces amongst one another may vary, as well as whether forces are fixed or time-varying. As an example, for a first routine (e.g., sit to stand), two forces may be applied in the vertical and lateral, but not forward, direction, whereas in the overground routine forces may be applied in the vertical, lateral, and forward directions. In another example, for a first routine (e.g., sit to stand), the vertical force and lateral forces may be time-varying based on sensor feedback, whereas in the overground routine a substantially constant vertical force may be applied with a time-varying forward and lateral forces each adjusted in real-time based on sensor feedback during execution of the routine. Thus, identifying the set of forces to apply to the subject may be specific to the particular training routine desired.

Accordingly, proceeding to 1005, method 1000 may include acquiring training routine-specific data related at least in part to parameters involving movement or posture for performing the selected training routine, in order to determine a first force to apply to the subject for the particular training routine. In one example, acquiring training routine-specific data may include acquiring kinematic (e.g. 127) and kinetic (e.g. 129) data from the subject while the subject is performing a task related to the particular training routine. As discussed above, in one example kinematic data may be acquired via sensors or markers (e.g. 906) placed strategically on a body of the subject, and kinetic data may be acquired via one or more ground force plate(s) (e.g. 909) which the subject may position their feet on during the acquiring kinetic data. In some examples, acquiring training routine-specific data may additionally or alternatively include electromyographic data from the subject. In some examples, acquiring training routine-specific data may include a physician or clinician empirically evaluating the subject for things such as range of movement, strength, response-time, or other variables related to movement for a specific routine. Furthermore, acquiring training routine-specific data at 1005 may include acquiring said data while a range of forces are applied to the subject, to determine how said force affects one or more parameters related to the specific training routine, which may be used in order to determine the first force to apply to the subject for the particular training routine. Such an example may include selecting a force to be the first force out of the range of forces tested, such that the first force comprises a force for which one or more of kinematic, kinetic, empirical, and/or EMG data most closely resembles data from a healthy individual (e.g. without a neurological disorder). In some examples, electrical stimulation (e.g. epidural and/or subdural stimulation) may be provided to the subject while acquiring training routine-specific data. For example, training routine-specific data may first be acquired in an absence of electrical stimulation, and then acquired in the presence of electrical stimulation. Such information may help to guide a selection of forces to apply to the subject for the particular training routine.

Thus, obtaining the first force may comprise a physician or clinician, or other user, selecting the first force from a range of forces presented on a user interface, said forces possibly being based on testing, where the first force may comprise a force for which the kinematic, kinetic, and/or EMG data most closely resembles data from a healthy individual. Alternatively, the control system may automatically select the desired force based on patient medical records, past test results, etc. Such examples may further comprise an experimentally-determined first force. Additionally or alternatively, data acquired from the subject (kinematic, kinetic, EMG, empirical, etc.) may be fed into a model (e.g. artificial neural network 315), which may generate a recommendation for the first force. The model may take into account one or more of kinetic, kinematic and/or EMG activity in order to predict the first force in some examples. In other examples, only kinetic and kinematic activity may be used via the model to predict the first force. In some examples where the first force is determined via the model (e.g. 315), the first force may comprise a force that is within a threshold (e.g. 5%) of the experimentally measured first force. In some examples (e.g. walking overground), the first force may comprise an upward force, for example.

Subsequent to obtaining the first force, method 1000 may proceed to 1015, and may include setting a second force, and in some examples, may further include setting a third force to apply to the subject. More specifically, it may be understood that the first force may in some examples result in altered parameters related to particular movement associated with the particular training routine. In one example including walking overground, the altered parameters may relate to walking speed, step length, alterations in energy exchange, posture (e.g. tilting), etc. However, the altered parameters may in other examples not be limited to walking overground, but may instead relate to an aspect of cycling (lateral sway, leg extension, etc.), postural parameters in going from a sitting to standing position or from a standing to sitting position, etc. As the first force may result in an alteration of certain parameters specific to the rehabilitation procedure, it may be desirable to compensate said first force via another force or forces (e.g. second and/or third force). Thus, at 1015, method 1000 may include simulating a desired behavior (e.g. gait) using a model (e.g. 401), taking into account the first force obtained at 1005, to determine the second force and/or third force to apply to the subject, to compensate any undesirable aspects (e.g. reduction of walking speed, step length, etc.) stemming from the first force. In an example where the model includes a model for gait, a passive walker model may be utilized, comprising an inverted pendulum-like gait behavior, where gravity may support continuous, alternating oscillations of limbs (e.g. 403a, 403b). In such an example, optimization parameters may include spring stiffness (e.g. 450), damping (e.g. 452), strike angle (e.g. 454), etc. However, it may be understood that the model at 1015 may not be restricted to a model for gait, but may instead comprise a model for cycling, going up or down a staircase, siting-to-standing, standing-to-sitting, etc. In such examples, the model may utilize the obtained first force to compute a compensation force or forces such that any undesirable aspects (e.g. undesirable or unexpected particular features of motion for the particular activity), may be reduced or avoided. In some examples, the second force may comprise a forward force, and the third force may comprise a lateral force (see inset 970 at FIG. 9).

Responsive to the first, second, and/or third forces being determined, method 1000 may proceed to 1020 and may include conducting gravity-assisted training procedures as a function of the first, second and/or third forces, including applying said forces via an actuation system, such as the example cable/motor system. More specifically, the first, second, and/or third forces may be applied to the subject via the robotic support system, such that the subject is supported during the training procedures in such a way as to assist or encourage improved performance from the subject in terms of parameters related to the training procedure. For example, in an example where the training procedure includes walking overground, such parameters may include step height, walking speed, step length, EMG activity, center of mass (CoM) trajectory, foot strike angle, etc. In other examples where the training procedure does not include walking overground, other relevant parameters may be used to assess overall performance during the particular training procedure. As explained herein, the forces applied may be adjusted in real-time responsive to sensor feedback an varying commands. In one example, based on the selected routine, desired forces are determined Thus, method 1000 depicted at FIG. 10 illustrates a high-level example method for setting a plurality of forces to apply to the subject while the subject is performing a rehabilitation procedure.

The application of a plurality of forces (e.g. two or more) may result in a force vector when the individual forces are added, which may comprise a desired force vector to apply to the subject over the course of the rehabilitation procedure. However, in real-world situations any number of issues may arise during a rehabilitation procedure that may result in the desired force vector becoming non-optimal or non-desired due to changes or unexpected behavior of the subject. In such an example, the subject may be monitored via a variety of parameters (discussed in detail below), such that one or more of the first, second, and/or third force may be adjusted in order to ensure that the forces applied to the subject are properly assisting a desired movement for the given procedure.

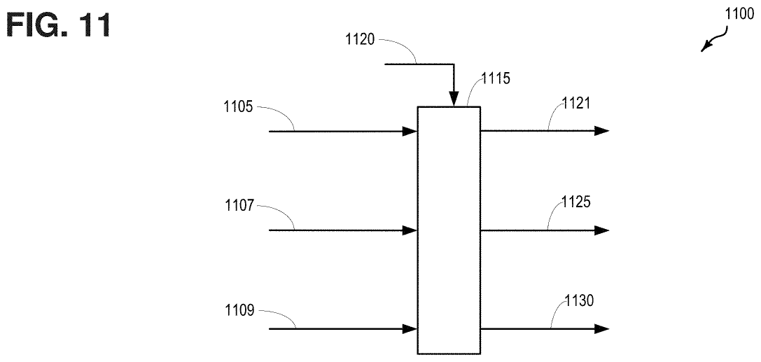

Accordingly, turning to FIG. 11, an example of a control system 1100 for monitoring and controlling forces applied to a subject during a rehabilitation training procedure is shown, where one or more of a first force, second force, or third force is applied to the subject, and where one or more of the first, second, and/or third forces may be manipulated during the procedure to facilitate a desired movement for the procedure. For example, relative magnitudes of one or more forces may be adjusted during overground movement of the patient based on sensor feedback and/or other adjusted commands such as based on the model described herein.

More specifically, as discussed above, a first force 1105, a second force 1107, and a third force 1109 may comprise forces determined by method 1000 as desired forces for a gravity-assist for a particular rehabilitation procedure. While the subject is conducting a particular procedure and the first, second, and/or third forces are applied to the subject, one or more parameters 1120 may be monitored. In an example, the monitored parameters 1120 may be parameters related to motion or movement of the subject (e.g. kinematic activity, kinetic activity, electromyographic activity), forces applied by the subject to the ground, other commands by the subject (such as a voice command like “walk forward” indicating a desired forward walking operation and possibly a desired forward velocity such as (“slowly” or “quickly”). In some examples, the monitored parameters may include a measurement of force applied to the subject, as monitored via an inertial measurement unit (IMU) (e.g. 915). The monitored parameters may in some examples include a measurement of force the subject is exerting on the robot, as monitored via the IMU. One or more of monitored parameters 1120 and the forces (e.g. desired forces) applied to the subject may be used to populate a model 1115. Based on input to the model, a second set of desired forces may be determined, and one or more motor commands (1121, 1125, 1130) to facilitate the second set of desired forces may be output from the model. In some examples, the model may comprise aspects of a neural network model (e.g. 315), and may further comprise aspects of gait or other desired movement sequences (e.g. walking overground, standing to sitting, sitting to standing, etc.), as discussed above with regard to method 1000. Based on output from the model, motor commands for controlling motorized actuators (e.g. winches) for controlling cable tension corresponding to updated desired forces to apply to the subject may be determined, where the updated desired forces may comprise an updated first force, updated second force, and updated third force. Thus, updated forces may be applied to the subject in order to assist the subject as desired according to the modeled parameters. It may be understood that the first, second, and third forces may be continually updated in this fashion for a duration of the rehabilitation training procedure. In this way, forces applied to a subject may be reliably controlled via a robotic support system (e.g. 900) such that the subject may be properly assisted during the rehabilitation procedure.

Regarding the application of desired forces to the subject, as discussed above, motorized winches (e.g. 912) may be used to control one or more force(s) on the subject via a plurality of cables (e.g. 913). In an example where one or more of a desired (e.g. determined via method 1000) first force, second force, and/or third force are applied to the subject, the forces may add such that force applied to the subject may comprise a resultant force vector. The resultant force vector may be measured via an IMU (e.g. 915) positioned in a node (e.g. 914) where intersection of the cables occur, for example. Such IMU measurements may be input into model 1115 as discussed (along with other parameters related to motion), which may enable real-time control of the forces (e.g. first, second, third) applied to the subject. Real-time control of the forces applied to the subject in such a way may be understood to comprise continuous input from monitored parameters as discussed, and continuous output commands to the motorized winches or actuators, during the course of a rehabilitation training program, with minimal delay between each input-output cycle (e.g. less than 1 second).

Regarding the parameters of motion utilized to populate the model 1115 (in addition to or alternative to forces monitored via the IMU), such parameters may include a center of mass trajectory, which may be used via the model 1115 to establish variations between an expected or desired CoM trajectory, and actual CoM trajectory. Similarly, a plurality of markers (e.g. 906) may be used in conjunction with a motion capture system (e.g. 903) to establish variations in between expected and actual movement patterns. If the input to the model indicates that expected versus actual modeled parameters do not coincide within a target threshold (e.g. within 5% or less) of the modeled pattern of movement, then one or more forces applied to the subject may be modified accordingly to properly assist the subject as desired. In some examples, the pattern of movement may include features such as leg excursion angle (deg), stride length (as % body height), step height (as % body height), speed (m/s), etc. Such features, including CoM trajectory, may in some examples, be determined as a function of movement patterns generated via the plurality of markers (e.g. 906).

In other examples, parameters of motion utilized to populate model 1115 may comprise ground reaction forces, which may be monitored via one or more ground force plates (e.g. 909). Alterations in ground reaction forces may be input to model 1115, which may result in modification of applied forces (e.g. first, second, third), to compensate said alterations in ground reaction forces.

In some examples, muscle activity may be monitored while the subject is conducting the rehabilitation procedure. As discussed above, muscle activity may comprise recorded electromyographic activity (e.g. 128). In one example, EMG activity may comprise activity of leg muscles, however an anatomical source of EMG activity may vary in some examples depending on the rehabilitation procedure being conducted. As an example, recorded EMG activity may in some examples indicate that the gravity-assist (e.g. first, second, and third forces) has become non-optimal or non-desired due to deviations in recorded muscle activity compared to expected activity. Accordingly, the model may compensate for such discrepancies by outputting updated forces to apply to the subject.

Thus, output from model 1115 may comprise first motor commands 1121, second motor commands 1125, and third motor commands 1130. In example system 1100, it may be understood that first motor command 1121 may comprise motor commands to control or regulate the first force applied to the subject, second motor command 1125 may comprise motor commands to control or regulate the second force applied to the subject, and third motor command 1130 may comprise motor commands to control or regulate the third force applied to the subject. In other examples, multiple motors may combine together to generate the first, second, and or third forces applied to the subject. As the robotic support system (e.g. 900) may comprise a support system using cables designed to facilitate application of forces in a plurality of directions within constraints of a 3D Cartesian coordinate system, the first, second, and third output motor commands may in some examples be interpreted via the controller (e.g. 935) to coordinate the three motor commands into a series of control steps to facilitate application of the updated first, second and/or third forces. More specifically, the motorized winches may be controlled based on the motor commands outputted from model 1115, such that the updated first, second, and/or third forces may be readily applied to the subject. To ensure that the desired forces are applied to the subject, the IMU (e.g. 915) positioned in the node (e.g. 914) where intersection of the cables occur, may be used to monitor forces applied to the subject and forces the subject exerts on the robotic support structure. When the updated desired forces match the IMU-measured force(s) within a threshold (e.g. within 5% or less, or within 1% or less), it may be understood that the support system is applying the appropriate corrective forces to the subject.

In some examples, controlling the motorized winches to apply desired forces on the subject based on model 1115 may comprise accounting for cable tension, which may further comprise accounting for inherent elastomeric properties of the cables. For example, built into model 1115 may include models pertaining to stiffness of the cables as a function of motor commands via the motorized winches, and may further be a function of forces exerted on the cables via the subject. Model 1115 may additionally include compensating for momentum of the subject in any particular direction. Parameters inputted into model 1115 may enable a determination of momentum of the subject, which may comprise a momentum vector quantity. For example, a rate of change observed via the markers (e.g. 906) strategically placed on the subject may in some examples enable a determination of momentum via a program stored on a memory of the controller. Momentum quantity may in some examples additionally or alternatively be determined at least in part via forces monitored via the IMU (e.g. 915). If variables such as cable tension, forces exerted on the robot via the subject, momentum of the subject, etc., are not accounted for, any calculations of motor commands to facilitate application of the first, second, and/or third forces may be insufficient. By accounting for such variables, appropriate motor commands may be applied via the robotic support system, such that appropriate updated first, second, and/or third forces may be appropriately applied to the subject.

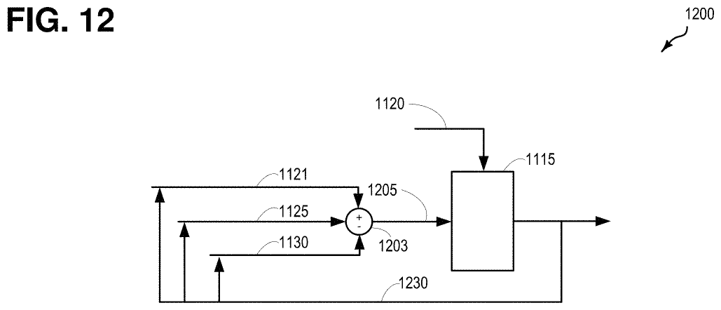

Turning now to FIG. 12, it illustrates another example embodiment 1200 of the system depicted at FIG. 11. More specifically, FIG. 12 illustrates how feedback from monitored parameters (e.g. parameters of motion related to a subject and/or measured forces from the robotic support structure as discussed with regard to FIG. 11) may be utilized to continuously update forces applied to a subject during a rehabilitation procedure. Accordingly, illustrated at FIG. 12 is the three motor commands exemplified by numerals 1121 (e.g. first motor command), 1125 (e.g. second motor command), and 1130 (e.g. third motor command). Additionally illustrated is model 1115, and monitored parameters 1120 discussed above. As discussed above at FIG. 11, first motor commands 1121, second motor commands 1125, and third motor commands 1130 may comprise motor commands to regulate the first, second, and/or third forces applied to the subject, respectively or when combined together control the forces as desired. Accordingly, such forces may be summed at junction 1203, to provide a resultant force vector 1205. The force vector 1205 may comprise a sum of forces applied to the subject, such that each of the desired first, second, and third force(s) are satisfied. As discussed above, an IMU (e.g. 915) corresponding to the robotic support system (e.g. 900) may be used to monitor forces applied to the subject until the sum of forces (exemplified via force vector 1205) is indicated as being applied to the subject, where when such conditions are satisfied it may be understood that the desired first, second, and third forces are being applied to the subject. With the desired first, second, and third forces applied to the subject, the monitored parameters 1120 (including but not limited to CoM trajectory, ground reaction force measurements (e.g. 129), features of motion (e.g. 127), muscle activity (e.g. 128), monitored forces via an IMU (e.g. 915), etc.) may be input to model 1115. Based on the applied forces and monitored parameters, it may be established whether assistive force may be applied to satisfy model 1115. Thus, model 1115 may output updated motor commands (e.g. 1121, 1125, 1130) as a function of feedback 1230 from model 1115. In this way, the system may enable forces applied to the subject to assist a desired movement pattern for the subject during the rehabilitation procedure.

Thus, based on the description of FIGS. 11-12, it may be understood that the robotic support structure is not directly controlling a position of the subject, but rather is controlling a force or set of forces applied to the subject to assist a desired movement routine.

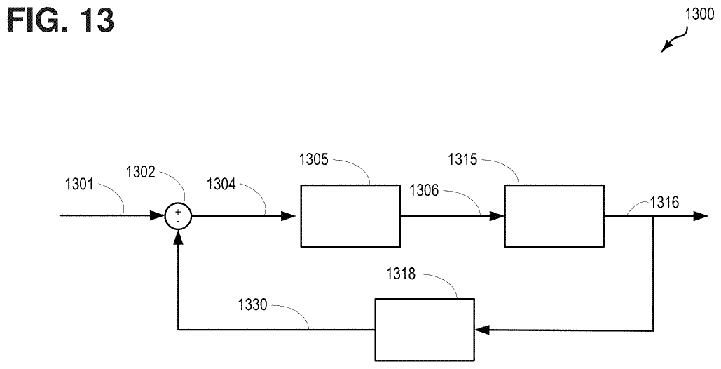

There may be other control schemes that the robotic support structure may employ. For example, in some cases it may be desirable to control the robotic support structure in a constant force mode, where the first, second and/or third forces are controlled to be substantially constant (e.g. within 5% or less of desired first, second and/or third forces) during a rehabilitation training procedure. Turning now to FIG. 13, it shows an example control scheme 1300 that may enable forces (e.g. first force, second force, third force) applied to the subject to be maintained at a substantially constant level for the duration of a rehabilitation procedure. Parts of such a control scheme may be stored on a memory of a controller (e.g. 935) which, when executed may enable forces applied to the subject to be maintained substantially constant. Briefly, a desired force vector 1301 (e.g. 1205) or desired individual forces (first, second, and/or third) may be input to summation node 1302, along with feedback 1330 from sensor(s) 1318. In an example where the control scheme is set up to control forces applied to the subject in a constant force mode, the sensor(s) 1315 may comprise sensors associated with an IMU (e.g. 915), such as those including but not limited to accelerometers (e.g. 916), gyroscopes (e.g. 917), magnetometer (e.g. 918), where the IMU measures forces from a robotic support system (e.g. 900) as applied to the subject, and vice versa. Output 1304 (e.g. error) from summing the desired forces to be applied to the subject with the feedback from sensors 1318, may comprise input to a proportional, integral, derivative (PID) controller 1305 (e.g. 935), the output of which may comprise motor commands 1306 to control forces applied to the subject via the robotic support structure 1315 (e.g. 900) to satisfy the model. Output from the robotic support structure may comprise a process variable 1316 (forces applied to subject), which may thus be monitored by sensors 1318, and again the error 1304 between output from sensors 1318 and the desired forces 1301 may be input to the PID controller 1305 to obtain new motor commands 1306 for controlling forces to satisfy the model.

Similar to that described above for FIGS. 11-12, the control scheme depicted at FIG. 13 may account for cable tension, momentum of the subject, etc.

In this way, it may be understood that the robotic support structure may maintain a substantially constant level of force (e.g. one or more of first force, second force, and/or third force) applied to the subject for all Cartesian directions for a duration of a particular training routine. In some examples, the desired forces may comprise forces determined as a function of method 1000, depicted at FIG. 10.

Without reiterating a similar diagram numerous times, such a control system 1300 as depicted may enable robust control over other desired variables. For example, it may be desirable to control forces on a subject via the robotic support structure (e.g. 900) such that a subject may be assisted to progress (e.g. overground) at a substantially constant velocity for a duration of a particular training routine (either part of the training routine or all of the training routine). In such an example, input to a summation node (e.g. 1302) may comprise a desired velocity of the subject, and a measured velocity. The error or offset may thus result in the robotic support structure applying forces (e.g. any combination of first, second, third forces) to control velocity of the subject to the desired velocity. In such an example, it may be understood that a control scheme such as that depicted at FIG. 13, and tailored for controlling a subject to a constant velocity, may be utilized. Sensors (e.g. 1318) for determining velocity may in some examples include sensors capable of obtaining information related to kinematic, kinetic and/or electromyograpic activity, from which velocity may be calculated via a program stored on a memory of the controller (e.g. 935).

Such a control scheme (e.g. 1300) may enable robust control over other variables in some examples. For example, forces may be controlled or such that particular variables such as step height, range of motion for one or more limbs of the subject, stride length, pedal velocity (in the case of a training program involving a bicycle), may be assisted. For example, sensors (e.g. 1318) may comprise kinematic, kinetic and or EMG feedback, which may be compared to desired variables such as step height, etc., and the output from such a comparison may comprise motor commands to encourage or assist the subject in achieving, for example, desired step height.

Thus, in summary, it may be understood that in some examples, forces applied to the subject may be part of a closed-loop, such that forces applied may be a function of one or more of monitored variables from parameters related to motion of the subject, measured forces acting on the subject, measured forces acting on the support structure via the subject, etc.

Turning now to FIG. 14, a high-level example method 1400 is shown for controlling a plurality of forces applied to a subject conducting a rehabilitation routine. More specifically, a first set of forces comprising a first force, second force, and/or third force may be applied to the subject, where the first set comprises a set of forces derived from at least a model of movement for a particular rehabilitation procedure. During the course of the rehabilitation procedure, one or more of force data (e.g. via the IMU), kinematic, kinetic, and/or muscle activity may be recorded, and processed via a controller to indicate whether assistive forces (e.g. a second set of first, second, and/or third forces) may be applied to the subject in order to properly assist a subject's movements corresponding to the particular rehabilitation procedure. Accordingly, such aspects may be carried out at least in part, via a program stored on a memory of a controller (e.g. 935) according to the method below. A control scheme to carry out the method may comprise, at least in part, control schemes such as that depicted at FIGS. 11-12. It may be understood that such a method may be applied to different types of rehabilitation procedures, as the method comprises a general example methodology and as such, may extend from step 1020 of method 1000 depicted above.

Method 1400 begins at 1405 and may include applying the first set of forces to the subject commencing a rehabilitation training procedure. As discussed above with regard to FIG. 10, in some examples one or more of kinetic (e.g. 129) and kinematic (e.g. 127) data may be recorded from the subject, and the data may be submitted to a neural network (as one example) to obtain a first force to apply to the subject (see step 1005 of method 1000). Subsequently, a second and/or third force may be determined via simulating a desired behavior (e.g. gait), where the second and/or third force may compensate any undesirable aspects of movement stemming from the first force (see step 1015 of method 1000). As an example where the rehabilitation training procedure includes walking overground, first force may comprise an upward force. The identified upward force if applied alone may result in undesirable aspects of movement (e.g. undesired tilting, reduction in walking speed, degraded stepping pattern, etc.). Accordingly, simulating gait for the overground rehabilitation training procedure as a function of the first force may provide second and/or third forces which may compensate the undesired aspects of movement, as discussed above at FIG. 10. In other examples where the rehabilitation training procedure includes other activities such as cycling, standing from a sitting position, sitting from a standing position, etc., the first, second, and/or third forces may differ from that described for an overgound walking procedure.

Thus, at 1405, applying the first set of desired forces may include the controller (e.g. 935) commanding or actuating the motors (e.g. 912) coupled to the plurality of robotic support system cables in a coordinated fashion such that cable tension is controlled so that the first set of desired forces (e.g. first force, second force, third force) may be applied to the subject. More specifically, the motors may be controlled such that tension in the cables are coordinated to apply the first force, second force, and third force to the subject. As discussed, a force vector may comprise a sum of the first, second, and third forces, whereby an IMU positioned at an intersection of the plurality of cables may enable indicating when the desired first set of forces are applied to the subject.

With the first set of forces applied to the subject, method 1400 may proceed to 1410 and may include monitoring one or more of at least kinematic and/or kinetic activity from the subject. At 1410, method 1400 may further include measuring forces applied to the subject, and forces applied via the subject on the support system, via sensors in the IMU (e.g. 915). In some examples, step 1410 may further include monitoring muscle activity (e.g. 128). As discussed above, such data may comprise features of movement extracted from monitoring markers (e.g. 906) strategically placed on the subject, forces related to particular movements (e.g. gait), CoM trajectory, etc. Such data may be communicated to the controller, where it may be processed at 1415 via a model (e.g. 1115) stored at the controller. The model may comprise expected features related to motion of the subject during the particular rehabilitation procedure, and the model may thus compare actual data recorded at 1410 to the model, such that any discrepancies between expected and actual features of movement may be indicated.

Proceeding to 1425, method 1400 may include determining compensatpry or assistive force(s) to apply in order to satisfy the model (e.g. 1115). As discussed above with regard to FIGS. 11-12, the model may output a second set of forces to apply to the subject, such that the monitored parameters of movement and/or forces more closely align with the modeled parameters. As discussed above, such a model may take into account momentum of the subject (which may be defined as a momentum vector quantity), and which may further account for elastomeric nature of the cables. The output (e.g. second set of forces) may include motor commands related to the first force, second force, and third force, for which the motors may be controlled to achieve via controlling tension in the support system cables. Accordingly, proceeding to 1430, method 1400 may include commanding the motors to apply such forces, such that the second set of forces are applied to the subject. The motors may be controlled until forces as measured via the IMU are substantially equivalent to the second set of forces as identified at step 1425. As an example, it may be determined that in order to encourage features of movement to more closely align with the modeled features, the first force may be decreased by a particular amount, while the second force may be increased via a defined amount, and the third force may remain constant. As another example, the first force may be increased a defined amount, the second force may be increased another defined amount, and the third force may be decreased yet another defined amount. Any permutations of such examples are within the scope of this disclosure.

Subsequent to actuating the motors to apply the second set of forces to the subject, method 1400 may proceed to 1435. At 1435, method 1400 may include indicating whether the particular training routine is ended (yes) or not (no). For example, in a case where the training routine comprises an overground walking routine, the end of the routine may be indicated when the subject gets to the end of a defined distance. In a case where the training routine comprises another routine, such as cycling, the routine may end after a predetermine timeframe or other relevant parameter. In other words, for each particular training procedure, there may be a point where the training procedure has ended, at which point method 1400 may end. If, at 1435 it is indicated that the training routine has not ended, method 1400 may return to 1410, and may include continuing to monitor relevant parameters of movement as discussed, such that yet another set of assistive forces may be applied to the subject. In this way, such forces may be continually updated and applied to a subject participating in a training routine in real-time (e.g. with minimal delay between input, such as parameters related to movement, and output, such as motor commands) such that a subject may be properly assisted (e.g. assisted as desired or expected) during a particular training routine.

Dashed box 1440 illustrates steps during method 1400 during which neuromodulation may be applied to the subject, as discussed above with regard to the description of FIG. 9. Neuromodulation may comprise either electrical stimulation (e.g. epidural and/or subdural electrical stimulation), or electrical stimulation and some form of pharmacological stimulation. For example, neuromodulation may be applied to the subject in order to facilitate desired movement by the subject. In cases where the subject is instructed to use lower limbs (e.g. walking overground, sitting-to-standing, standing-to-siting, etc., lower limb stimulation may be provided. Electrical stimulation sites may comprise one, and may in some examples be turned on or off depending on specific sub-phases of a particular routine. Electrical stimulation sites may alternatively comprise two, where each stimulation site may be independently turned on or off depending on specific sub-phases of a particular routine. Electrical stimulation may be time-specific in some examples, whereas in other examples the stimulation may be in real-time (e.g. minimal delay (e.g. 5 seconds or less, or 1 second or less) between input, such as parameters related to movement, and output, such as electrical stimulation) as a function of recorded parameters related to motion (e.g. monitored kinematic, kinetic, neuronal activity and/or EMG activity). Details regarding the application of neuromodulation to the subject have been described above at FIG. 9.

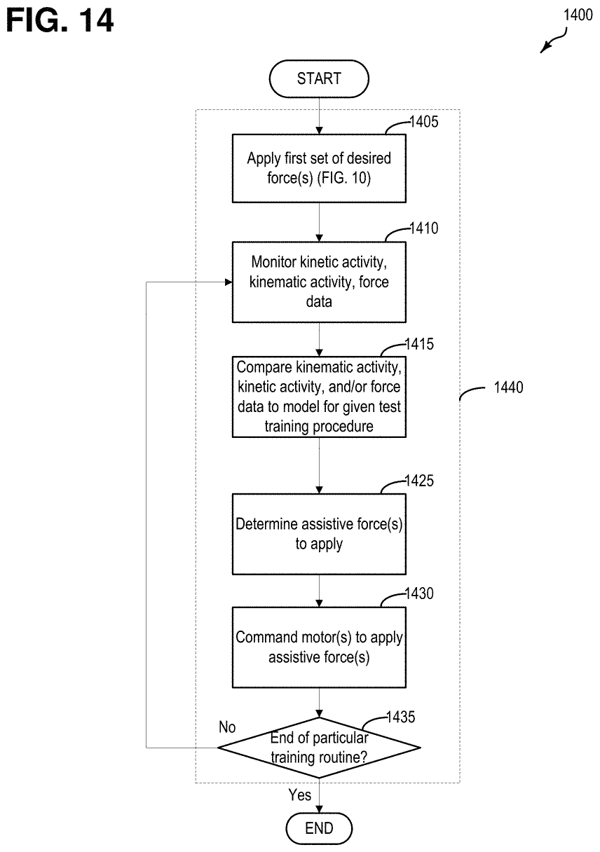

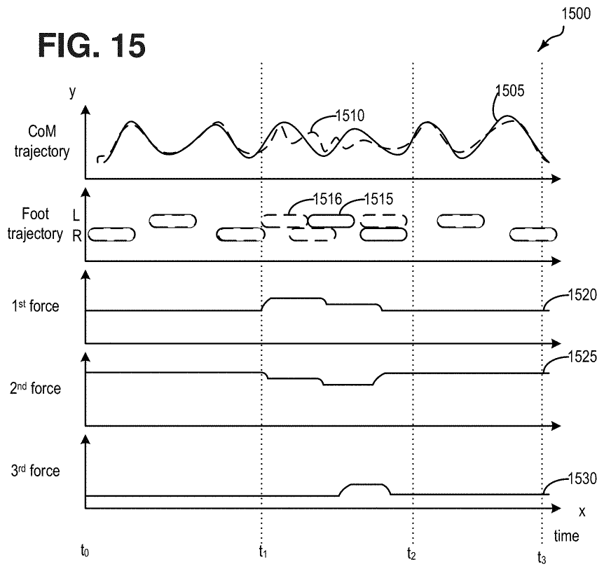

Example embodiments for uses of the robotic support structure (e.g. 900) discussed herein, are now elaborated. Turning to FIG. 15, an example timeline 1500 for conducting an overground training routine, using the methods and systems discussed herein, is shown. Timeline 1500 includes plot 1505, indicating a center of mass (CoM) trajectory of a subject undergoing the overground training routine with assistive force corrections or compensations as discussed above with regard to FIG. 14, over time. Plot 1510 indicates a CoM trajectory of the subject if assistive force corrections or compensations are not employed, over time. Timeline 1500 further includes foot trajectory 1515, over time. Foot trajectory 1515 illustrates right (R) and left (L) foot placements. Dashed foot placements 1516 illustrate degraded foot placements which may occur if assistive force corrections or compensations are not applied. Timeline 1500 further includes plot 1520, indicating an amount of a first force applied to the subject, plot 1525, indicating an amount of a second force applied to the subject, and plot 1530, indicating an amount of a third force applied to the subject, over time. For plots 1520, 1525, and 1530, a greater amount of force corresponds to a higher position on the y axis. In this example timeline 1500, it may be understood that the first force comprises an upward force (vertical) on the subject, second force comprises a forward force (horizontal) on the subject, and third force comprises a lateral force on the subject.

At time t0, the subject initiates an overground training routine. It may be understood that at time t0, the subject is standing still, without engaging in an attempt to walk. Accordingly, a set of forces (plots 1520, 1525, 1530) are applied to the subject (see step 1405 of method 1400), to assist the subject. More specifically, the set of forces may be determined according to method 1000 depicted at FIG. 10, for the particular selected training routine (e.g. overground). With the forces applied to the subject via the robotic support structure (e.g. 900), between time t0 and t1, the subject proceeds with walking overground, while the robotic support system assists the movement. While the subject is traversing overground, it may be understood that one or more of kinematic activity (e.g. 127), kinetic activity (e.g. 129), and/or muscle activity (e.g. 128), may be recorded via the controller (e.g. 935). Furthermore, forces applied to the subject via the support system, and forces the subject is exerting on the support system, may be monitored via an inertial measurement unit (e.g. 915). As discussed above, by obtaining such information, features of movement may be extracted. Such features may be incorporated into a model (e.g. 1115), where the model may comprise expected features related to motion of the subject. The model may be used to indicate any discrepancies between expected and actual features of movement. For simplicity, two such features of movement are illustrated, specifically CoM trajectory and foot trajectory.

Between time t0 and t1, it may be understood that the CoM trajectory of the subject is in line with the model. In other words, assistive alterations to the forces applied are not indicated, and thus the forces are held constant. Plot 1505 illustrates CoM trajectory with assistive force alterations (corrective or compensatory forces), and plot 1510 illustrates CoM trajectory if assistive alterations are not provided via the robotic support structure. As illustrated, the two CoM trajectories are substantially equivalent between time t0 and t1, indicating that the subject is performing as expected without assistive correction to the already applied forces. Similarly the two foot trajectories substantially overlap between time t0 and t1.

However, between time t1 and t2, the retrieved data acquired from the subject and fed into the model (e.g. 1115) indicates discrepancies between the model and the retrieved data. Accordingly, in response to such discrepancies, forces applied to the subject may be slightly altered, to encourage or assist the subject in performing the routine in an expected fashion. Thus, between time t1 and t2, each of the first force, second force, and third force are indicated to be altered. For example, the first force applied to the subject is increased, then decreased slightly, then decreased more between time t1 and t2. The second force is decreased, then decreased again, before increasing. The third force increases, and then decreases between time t1 and t2. If such assistive force alterations were not conducted (plot 1510), the CoM trajectory would be substantially different than a desired trajectory. Similarly, if assistive force alterations were not conducted (foot trajectory 1516), placement of feet of the subject may become non-optimal or desired. Accordingly, desired foot trajectory may more closely align with actual foot trajectory when assistive force alterations are provided (plot 1515).

Between time t2 and t3, once again no assistive force corrections are indicated via the model, and thus the subject proceeds with the forces being maintained constant or substantially constant.

It may be understood that timeline 1500 may comprise a snapshot of a training routine, where the training routine includes walking overground for a predetermined length. For example, there may be numerous times throughout such a training routine that the robotic support structure may alter forces applied to the subject, such that a desired pattern of movement of the subject is encouraged. Shown for simplicity is just one example of such a compensation.

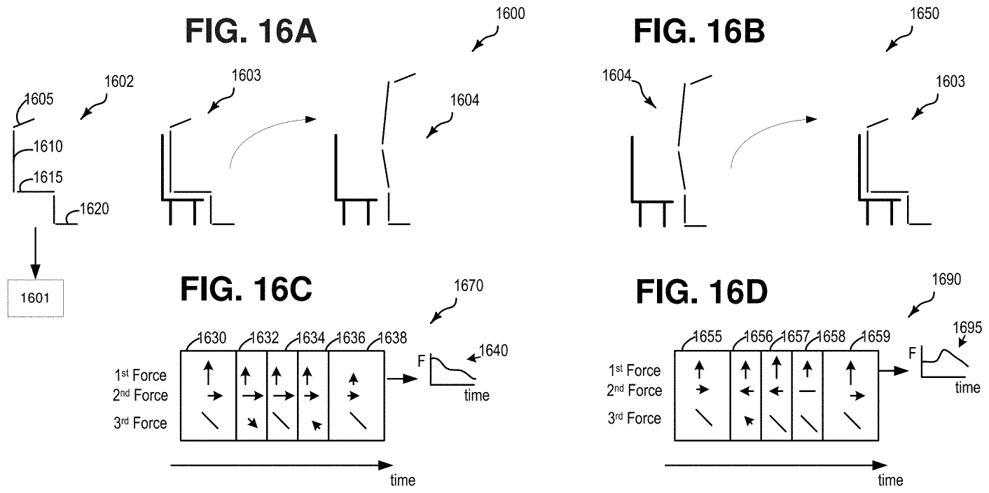

Turning now to FIGS. 16A-B a training routine may involve a subject going from a sitting 1603 to standing 1604 position 1600, or from a standing (1604) to sitting (1603) position 1650. Turning to FIG. 16A, a stick diagram of a person 1602 is shown. Stick diagram 1602 illustrates a person divided into relevant segments, including head 1605, trunk 1610, thigh 1615, and feet 1620. Such segments may be modeled during transitions between sitting 1603, and standing 1604, for example. More specifically, in one example, a healthy subject (e.g. absence of neurodegenerative disorder), may be fitted with one or more of markers (e.g. 906) for monitoring kinematic activity, bipolar electrodes for recording muscle activity, and may place their feet on ground force plates for measuring kinetic activity. The healthy person may be instructed to conduct sitting-to-standing (e.g. 1600) routines and/or standing-to-sitting (e.g. 1650) routines. Based on data acquired from the healthy subject, a model 1601 may be generated for a desired pattern of movement for the sitting-to-standing procedure and/or the standing-to-sitting procedure. The model may be used to incorporate data (kinematic, kinetic, electromyographic) acquired from a subject with a neurological disorder, in order to determine desired forces to apply to the subject during either sitting-to-standing or standing-to-sitting procedures as will be discussed below. In some examples, such a model may be generated for a particular subject based on movements of a healthy person that is of the substantially equivalent dimensions (height, weight) and build.

The general scheme of method 1000 depicted at FIG. 10 may in some examples be used to determine a first force to apply to the subject, and then to determine second and/or third forces to apply to the subject for a sitting-to-standing or standing-to-sitting routine. For example, at step 1005, method 1000 may include acquiring training-routine specific data from the subject. Turning to FIG. 16A, such data may include recording one or more of kinematic, kinetic, and/or electromyographic data from the subject while the subject is seated, and attempts to stand. Alternatively, turning to FIG. 16B, such data may include recording one or more of kinematic, kinetic and/or electromyographic data from the subject while the subject is standing, and attempts to sit. In some examples, a series of first forces may be applied to the subject, for example a series of upward forces, to assist movement. For example, the first forces may comprise upward forces equaling 20%, 25%, 30%, 35%, 40%, 45%, 50%, 55%, 60% (percent bodyweight), or less than 20% or greater than 60% in increments of 5%. The subject may be monitored while attempting to stand (FIG. 16A), or attempting to sit (FIG. 16B), and from an analysis of features related to movement of the subject (kinematic, kinetic, electromyographic), a desired first force (e.g. desired upward force) may be determined. In other words, the first force may be empirically determined in some examples. In still other examples, as discussed above with regard to FIG. 10, kinetic and kinematic data may be acquired from the subject while attempting to stand or sit, and the acquired data may be fed into a neural network (e.g. 315) which may output a desired or desirable (e.g. optimal) first force. Subsequently, a second and/or third force may be determined as discussed with regard to step 1015 at FIG. 10. More specifically, applying the first force may adversely impact some aspects of movement in going from a sitting to standing position, which may be compensated by applying a second and/or third force. For example, altered parameters may relate to a speed of movement of particular segments of the body, length of movements related to specific body segments, undesirable changes in ground force reactions (e.g. kinetics), undesirable tilt (e.g. horizontal and/or lateral sway), etc. Accordingly, desired behavior may be simulated using the model (e.g. 1601) described above, taking into account the obtained first force, to determine the second force and/or third force to apply to the subject, to compensate any undesirable aspects stemming from the first force.

In some examples, the first, second and third forces as described above may be utilized to conduct the training procedure for a duration of the training procedure. In other words, the forces to apply to the subject during either a sitting-to-standing (FIG. 16A) or standing-to-sitting (FIG. 16B) may be determined as described, and applied to the subject during the selected training procedure. In such an example, as discussed above and which will be discussed in further detail below, forces applied to the subject may still be compensated during the particular routine, if modeled parameters related to movement differ from recorded parameters of movement.

However, as there is a series of movements that may take place in going from a sitting to a standing position, and from a standing to seated position, one set of forces (which may be compensated throughout the selected training routine), may not be sufficient to satisfy all constraints related to a desired pattern of movement of the subject. Turning now to FIG. 16C, an example illustration 1670 is shown, illustrating a number of different “phases” where a desired first force, second force, and third force may change depending on what aspects of movement are being conducted by the subject in going from a sitting position to a standing position. Thus, it may be understood that FIG. 16C is discussed with reference to FIG. 16A. Similarly, FIG. 16D will be discussed with reference to FIG. 16B.

In the example illustration 1670, illustrated are a first phase 1630, second phase 1632, third phase 1634, fourth phase 1636, and final phase 1638. In practice, there may be any number of phases (greater than five or less than five) depending on a level of detail incorporated into the model (e.g. 1601) of movement. For example, as a level of detail in the model increases, the number of phases may increase, and vice versa. In other examples, the number of phases may be based on a defined set of movements that the subject undergoes in going from a seated to standing position, and thus may be fixed.

In example illustration 1670, five phases of movement are shown, as discussed. In this example, the five phases may refer to an initial seated position 1630, forward movement of the trunk/head 1632, thigh/leg/foot engagement 1634 and lifting of the trunk/head, extension/elongation of the body 1636, and final standing position 1638. Such phases are exemplary in nature, and other phases are within the scope of this disclosure.

For each phase in example illustration 1670, a first force, second force, and third force are illustrated. A length of the arrow for each force indicates a relative amount of each force. In this example, it may be understood that first force comprises an upward (vertical) force, second force comprises a forward (horizontal) force, and third force comprises a side-to-side (lateral) force. In cases where no force is applied, a line without an arrow is depicted.

In a case where different forces are determined for each phase of a routine, such as the routine depicted at FIG. 16C, general methodology discussed above with regard to FIG. 10 may still apply. However, the difference may be that for each phase, new first, second, and third forces need to be determined. It may not be practical at each phase to empirically test a range of first forces, as described above for an overground procedure, and thus modeling approaches may be utilized instead. For example, kinetic, kinematic and/or EMG data may be acquired from the subject while the subject is undergoing a sitting-to-standing routine, and the data may be fed into a neural network model (e.g. similar to 315), which may output a desirable first force for each phase of the routine. Similarly, given the first force for each phase, second and third forces may be determined by following similar logic as that described at step 1015 of FIG. 10. For example, the desirable first force for each phase may result in undesired movement parameters at each phase, which may be corrected via a second and/or third force. Furthermore, in some examples, the first force may not result in undesirable movement parameters for a specific phase, but rather a second force and/or third force may be desirable to encourage or assist a particular type of movement. For example, consider phase 1632, which comprises forward movement of the trunk/head. In such an example, a desired first force may or may not adversely impact other movements, but it may be further desirable to increase the forward force, to encourage or assist such a movement comprising the second phase. Similar logic may apply to each phase, and selection of forces therein.

Thus, second and third forces may be determined via simulating a sitting to standing routine, for example using model 1601 as discussed. By modeling the sitting to standing routine, and taking into account the first force applied to the subject (determined for each phase), second and third forces may be determined for each phase.

By way of explanation, an example using illustration 1670 will herein be discussed. While the subject is seated, the indicated first (upward), second (forward), and third (lateral) forces are applied via the robotic support system (e.g. 900). First force is greater than the second force, and the third force is not applied. Thus, the subject may be supported in an upright position due to the greater first force, but may be poised for attempting to stand, as indicated via the application of the second force. In some examples, the second force may not be to “ready” the subject for attempting to stand, but may instead counter an undesirable aspect of movement related to the applied first force.

It may be understood that during the routine, one or more of kinematics, kinetics, and/or muscle activity may be recorded from the subject, such that it may be readily indicated when the subject has entered into a new phase of the routine. For example, when kinematic data (or kinetic or EMG data) recorded from the subject indicates that a first phase is over, and a second phase has begun, motors associated with the robotic support structure may be controlled to actuate the determined forces for the second phase, and so on.

Upon transitioning to the second phase 1632, forces applied to the subject may be controlled to match the determined first, second, and third forces. As the second phase in this example includes forward movement of the trunk/head, the first (upward) force is reduced, and the second (forward) force is increased. Furthermore, such forces may introduce undesirable lateral tilt to the subject, which is countered by the third force. While the example illustrates an increase in second force, in some examples the second force may instead be decreased, to counter forward motion of the subject, which in the absence such a force, the subject may lean farther than desirable. Such examples are meant to be illustrative, as discussed.

The third phase 1634 may include thigh/leg/foot engagement and lifting of the trunk/head. In this example illustration, first force and second force may remain essentially the same as that applied in the second phase 1632, but due to the engagement of the legs, the lateral tilt no longer needs correction, and thus the third force is removed.

The fourth phase 1636 may comprise extension/elongation of the body. In this example, extension/elongation of the body may be accomplished as desired by maintaining the first force, and decreasing the second force. Furthermore, a third, lateral force, may be applied to encourage desired movement.

Finally, the fifth phase 1638 may comprise standing without forward or backward movement (or lateral movement). A reduced first force and reduced second force, along with removal of the third force, may comprise desirable forces for assisting standing in place.

Controls for each force (e.g. first force, second force, third force) may in some examples be smoothed such that forces applied to the subject readily transition from one phase to the next without abrupt changes in force applied. Inset 1640 depicts an example for the first force, which starts out being a higher applied force, but then drops and stabilizes over the second, third, and fourth phases, and further decreases in the fifth phase.

In an effort to avoid redundancy, illustration 1690 depicted at FIG. 16D will only be briefly described, as it is substantially similar to that described for FIG. 16C. For example, similar to FIG. 16C, there may be five distinct phases including first phase 1655, second phase 1656, third phase 1657, fourth phase 1658, and fifth phase 1659. First phase 1655 may comprise the subject standing still, while fifth phase 1659 may comprise the subject in a seated position. Phases 1656-1658 may comprise various phases of going from standing to sitting, similar to those phases described at FIG. 16C in going from sitting to standing. For each phase, a desired first force may be determined, in addition to second and/or third forces. In example illustration 1690, it may be understood that, like FIG. 16C, first force comprises a vertical force, second force comprises a horizontal force, and third force comprises a lateral force. Thus, with the subject standing in place, a first force and second force are applied to facilitate standing without undesired movements. Subsequently, as the subject transitions through the various phases of the standing to sitting routine, a separate set of forces are applied to the subject at each phase. Inset 1695 depicts an example of the change in first force overtime, similar to inset 1640 depicted at FIG. 16C.

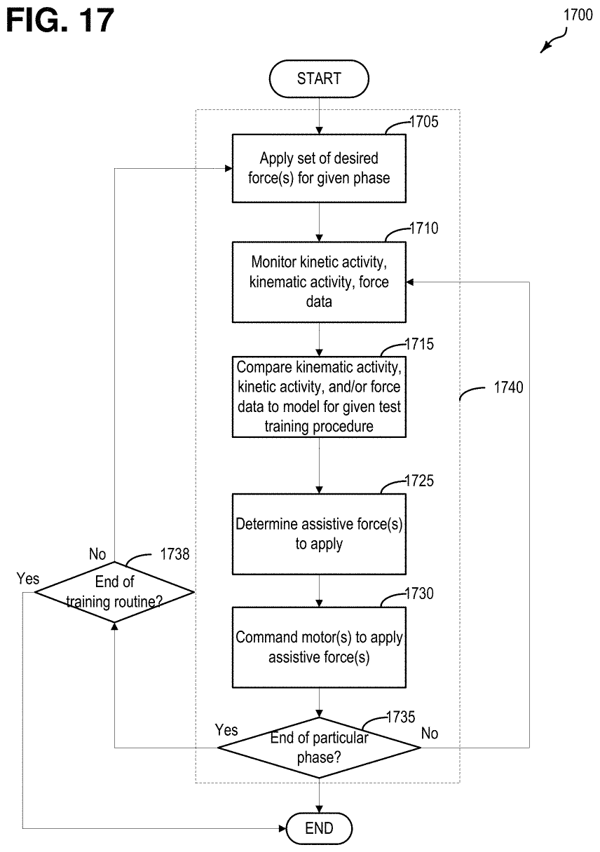

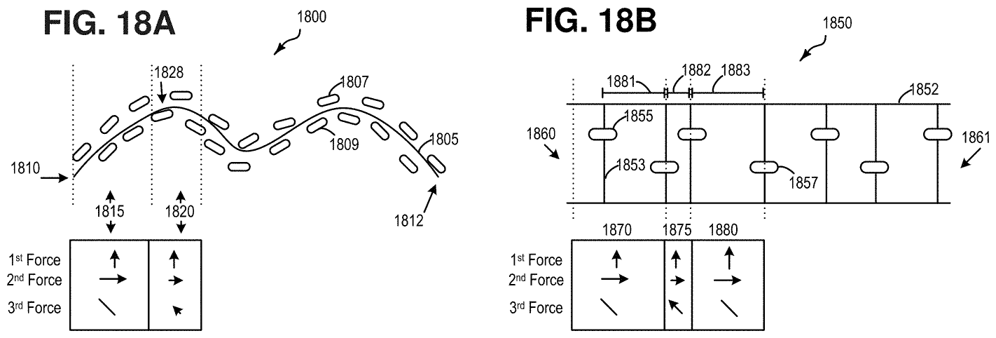

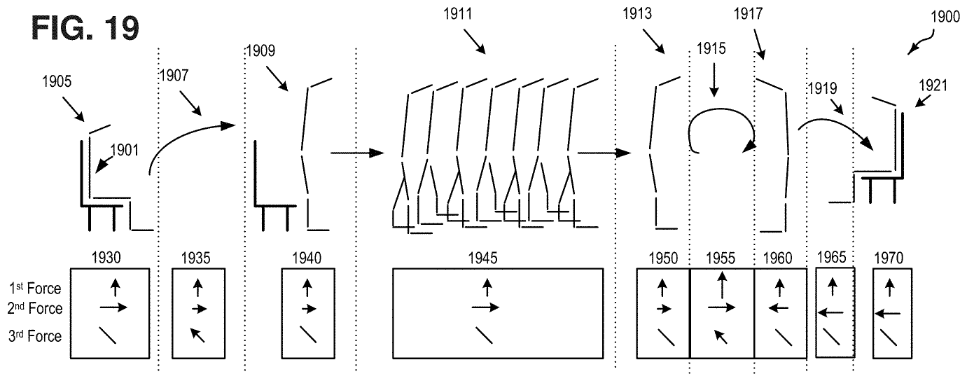

Regardless of whether one set of forces are carried through a routine in going from sitting to standing, or standing to sitting, or whether forces are altered as a function of what phase of movement a subject is conducting for a particular procedure, such forces may still be compensated to some degree based on monitored conditions related to movement, as discussed above with regard to FIGS. 11-12 and FIG. 14. Turning now to FIG. 17, a high-level example method 1700 is shown for conducting either a standing to sitting procedure, or a sitting to standing procedure, where such a procedure is divided into a plurality of phases such that different forces may be applied to the subject during each phase. Method 1700 may be carried out at least in part, via a controller (e.g. 935). It may be understood that method 1700 may not be limited to conducting the standing to sitting or sitting to standing procedure, but may include any routines that involve different phases where desired forces to apply to the subject may change (see for example FIGS. 18-19). Method 1700 is substantially equivalent to method 1400 depicted at FIG. 14, and thus will only be briefly described herein, while highlighting the differences. Method 1700 begins at 1705 and may include applying a set of desired forces (e.g. first force, second force, third force) for a particular phase. For example, turning to FIG. 16C, if the routine includes sitting to standing, and the subject is in the first phase, then a first and second force may be applied, while the third force is not applied as indicated at FIG. 16C. However, again, such forces are exemplary in nature.

Returning to FIG. 17, subsequent to applying the set of desired forces at 1705, method 1700 may proceed to 1710, and may include monitoring parameters related to movement (e.g. kinematic, kinetic, electromyographic) of the subject as the subject is performing the routine, as described herein. Data regarding the monitored parameters may then be fed into a model for the particular desired movement (e.g. 1601), which may output assistive forces at 1725 to apply to the subject, such that the desired pattern of motion is facilitated. Responsive to determining the assistive or compensatory forces to apply, method 1700 may proceed to 1730, and may include commanding motors (e.g. 912) to control one or more cables to apply the assistive forces on the subject. With the assistive forces applied at 1730, method 1700 may proceed to 1735, and may include indicating whether a particular phase has ended. Indication that a phase may be ended may include monitoring kinematic, kinetic and/or electromyographic data from the subject, to obtain information related to a particular phase of movement the subject is undergoing. If, at 1735, it is indicated that the particular phase is not complete, method 1700 may return to 1710, where parameters related to movement are continued to be acquired, such that additional assistive forces may be applied where desired.

Alternatively, if at 1735 it is indicated that a particular phase has ended, for example a first phase (e.g. 1630) has ended and a second phase has begun (e.g. 1632), method 1700 may proceed to 1738. At 1738, method 1700 may include indicating if the particular training routine has ended. As discussed above, a particular training routine may be indicated as being over when a subject completes a task, for example the subject reaches a standing position from a seated position, when a clinician or physician determines the task to be ended, after a predetermined time frame, etc. If it is indicated that the routine has ended, method 1700 may end. Alternatively, if at 1738, the training routine is not ended, then it may be determined that another phase of the routine has commenced, and thus the controller may commence applying the set of forces determined for that particular phase.

Dashed box 1740 illustrates steps during method 1700 during which neuromodulation may be applied to the subject, as discussed above with regard to the description of FIG. 9, and FIG. 14. As discussed, neuromodulation may comprise either electrical stimulation (e.g. epidural and/or subdural electrical stimulation), or electrical stimulation and some form of pharmacological stimulation. For example, neuromodulation may be applied to the subject in order to facilitate desired movement by the subject. In cases where the subject is instructed to use lower limbs (e.g. walking overground, sitting-to-standing, standing-to-siting, etc., lower limb stimulation may be provided. Electrical stimulation sites may comprise one, and may in some examples be turned on or off depending on specific sub-phases of a particular routine. Electrical stimulation sites may alternatively comprise two, where each stimulation site may be independently turned on or off depending on specific sub-phases of a particular routine. Electrical stimulation may be time-specific in some examples, whereas in other examples the stimulation may be in real-time as a function of recorded parameters related to motion (e.g. monitored kinematic, kinetic, neuronal activity and/or EMG activity). Details regarding the application of neuromodulation to the subject have been described above at FIG. 9.

Turning now to FIG. 18A, another example embodiment 1800 of a training routine is illustrated. More specifically, the training routine may comprise a subject walking overground on a curved path 1805. In one example, the curved path is a fixed path on a floor of a workspace of a robotic support system (e.g. 900). In another example, the curved path may comprise an augmented reality-based path. In still another example, the path may comprise a path communicated to the subject in a virtual reality setting. Illustrated are a subject's left foot 1807 and right foot 1809. The routine may have a starting point 1810, and an end point 1812. In some examples, forces applied to the subject may be held constant throughout the duration of the routine. In such an example, it may be understood that the robotic support system may be configured to apply a constant first force, second force and/or third force on the subject throughout the duration of the routine. An illustrative control system for maintaining a constant set of forces on the subject throughout the duration is shown at FIG. 13. In some examples, the subject may be controlled to a particular velocity. In such an example, forces applied to the subject may be controlled or regulated via one or more parameters related to movement of the subject, such that velocity may be determined. Such parameters may include kinematic, kinetic and/or EMG data from which velocity may be ascertained and forces controlled to maintain such a velocity.

In other examples, force on the subject may be controlled as a function of parameters related to movement of the subject, such that a model of movement (e.g. 1120) is satisfied, and forces applied to the subject are controlled accordingly. Illustrative control systems for controlling forces applied to the subject as such are described above at FIGS. 11-12, for example. In such an example, forces applied to the subject may slightly change as the subject is progressing throughout the routine, such that parameters of the model are satisfied.

In still other examples, as discussed above with regard to FIG. 16C-D, there may be sub-phases for a particular routine, where desired forces applied to the subject may change, or the feedback control settings for providing the desired force may be different (e.g., different control gains (PID), etc.). To illustrate this point, two example sub-phases 1815, and 1820 are shown at FIG. 18A, however it may be understood that there may be any number of sub-phases for a particular routine. As discussed above with regard to FIGS. 16A-D, desired forces (e.g. first force, second force, third force) for each sub-phase may be determined by methodology such as that depicted at FIG. 10, for example. In this example embodiment 1800, it may be understood that a first force (e.g. upward force), and a second force (e.g. forward force) may be applied to the subject in the first phase, while a third force (e.g. lateral force) may not be applied. As the subject enters the second phase 1820 which includes a turn 1828, the desired forces, may be different. As illustrated, forces applied to the subject for the second phase include a decrease in the forward force, and a slight lateral force in the direction indicated, to support the subject as the subject performs the turn. As discussed above, such forces for the sub-phases are illustrative.

Turning now to FIG. 18B, another example illustration 1850 of a training routine is shown. More specifically, illustration 1850 shows an irregularly spaced ladder 1852. In some examples, the ladder may comprise an actual ladder, while in other examples the ladder may comprise a projected ladder, for example. In other words, the ladder may comprise an augmented reality device in some examples. The ladder in other examples may be a communicated to the subject via a virtual reality setting, as discussed above. Illustrated are the subjects left foot 1855, and right foot 1857. Individual rungs of the ladder 1853 are illustrated. The routine may have a starting point 1860 and an end point 1861. As discussed above, control schemes for applying force to the subject may be include controlling the subject via a constant set of forces throughout the duration of the routine. In such an example, forces applied to the subject may be held constant via a control scheme such as that described at FIG. 13. In other examples, the subject may be controlled to a particular velocity, where forces applied to the subject may be controlled or regulated via one or more parameters related to movement of the subject (e.g. kinematic, kinetic, and/or EMG data), such that velocity may be determined.

In other examples, force on the subject may be controlled as a function of parameters related to movement of the subject, such that a model of movement (e.g. 1120) is satisfied, and forces applied to the subject controlled accordingly (see control schemes at FIGS. 11-12).

In still other examples, desired forces applied to the subject may differ between different phases of movement related to the training routine, as discussed. Briefly, desired forces for each particular phase may be determined, and applied accordingly to properly assist the subject. Illustrated for example are three phases 1870, 1875, and 1880. It may be understood that such phases are exemplary. Briefly, a first force 1870 (e.g. upward) and a second force 1875 (e.g. forward) may be applied to the subject in the first phase, when the space between the ladder rungs is a defined distance 1881. The second phase 1882 may comprise a shorter distance 1882 between rungs 1853, and thus the second force is decreased, and a third force is applied (e.g. lateral force) to properly assist the subject in progressing to the next rung in the second phase. Spacing between rungs may again increase in the third phase, thus the desired forces in the third phase may be similar to those described at the first phase. As discussed above, such depictions of forces to apply to the subject are illustrative in nature.

As discussed above, regardless of whether one set of forces are carried through particular routines, such as the routines discussed with regard to FIGS. 18A-B, or whether forces are altered as a function of different phases of movement related to the specific routines, such forces may still be compensated to some degree based on monitored conditions related to movement. Thus, in examples where particular phases are not included, method 1400 depicted at FIG. 14 may be utilized, whereas in examples where particular phases are included, method 1700 depicted at FIG. 17, may be utilized.

Turning now to FIG. 19, it depicts another example training routine 1900. Such a training routine may comprise a series of movements, and as such, it may not be desirable to employ a constant of substantially equivalent assistive force throughout the entire duration of the routine. Briefly, a subject 1901 may be in a seated position 1905, and then may transition 1907 to a standing position 1909. Once in the standing position, the subject may walk overground 1911 for a predetermine distance or duration, and at the end of the overground routine 1911, may again conduct a standing position 1913 (e.g. 1909). From the standing position 1913, the subject may rotate 1915 (e.g. 180 degrees), to a standing position 1917 that is a reverse direction as the standing positions indicated by numerals 1909 and 1913. From there, the subject may transition 1919 to a seated position 1921 (e.g. 1905).

Thus, there may be a number of phases to such a routine. For example, for the seated position, a first set of forces 1930 may be applied to the subject. As the subject transitions 1907 to the standing position 1909, a second set of forces 1935 may be applied to the subject. With the subject in a standing position 1909, a third set of forces may be applied to the subject. While walking overground 1911, a fourth set of forces 1945 may be applied to the subject. Subsequent to walking overground 1911, with the subject in the standing position 1913, a fifth set of forces 1950 may be applied to the subject. It may be understood that in some examples, the fifth set of forces 1950 may be the same as the third set of forces 1940. While the subject is rotating 1915 in order to sit down, a sixth set of forces 1955 may be applied to the subject. After rotation, a seventh set of forces 1960 may be applied to the subject. It may be understood that the seventh set of forces 1960 may comprise a substantially similar set of forces as that of the third set of forces 1940 and fifth set of forces 1950. From the standing position 1917, the subject may transition 1919 to a seated position 1921. An eighth set of forces 1965 may be applied to the subject as the subject is transitioning 1919 to the seated position. With the subject seated, a ninth set of forces 1970 may be applied to the subject. Further, the applied forces may be controlled to transition between the desired forces for different routines. In one example, the forces are transitioned concurrently and automatically, and in another example they are transitioned responsive to a user input and/or patient command/movement.

While not explicitly illustrated, it may be understood that for each part of the routine, for example transitioning from sitting to standing 1907, there may be any number of phases specific for that particular part of the routine, as discussed in detail above with regard FIGS. 16A-D.

For each part of the routine (including any phases for particular parts of the routine), forces applied to the subject may be held constant (see FIG. 13), may be manipulated to achieve a predetermined velocity of the subject (see FIG. 13), or may comprise forces that may subtly change based on a model or models of expected or desired parameters related to movement of the subject (see FIGS. 11-12, 14, 17).

Other routines not specifically illustrated may include climbing or descending a staircase, where the systems and methods described herein may be similarly utilized to assist or encourage the subject to conduct the particular routine.

An algorithm was developed that configures the assistance of trunk movements to restore an inverted pendulum-like gait behavior in neurologically impaired individuals despite the application of a bodyweight support tailored to their specific need. The gravity-assist establishes a safe and natural gait rehabilitation environment wherein individuals with neurological deficits are capable of performing basic and skilled locomotor activities that would not be possible without robotic assistance. The short- and long-term ameliorations of locomotor performance in response to gait rehabilitation with gravity-assist illustrate the potential of this environment to enhance motor recovery.

Partial bodyweight supported gait therapy is a common medical practice to improve locomotor recovery after neurological disorders (12, 13). Currently, therapists configure the level of bodyweight support empirically based on mere visual observations. As previously reported in rodent models of SCI (20), it was found found that the clinical phenotype of each neurologically impaired subject determined the precise amount of upward trunk support required to facilitate gait execution. In the most affected subjects, even minimal changes in the configuration of the upward force strongly affected the locomotor performance of the subject.

Moreover, current bodyweight support systems deliver trunk assistance restricted to the upward direction (18). Yet, it was found that the application of upward forces to the trunk induced a gradual backward shift in the orientation of the body, which considerably destabilized the control of standing and walking. The addition of well-calibrated forward forces was critical to restore the postural orientation of the body, and thus alleviate the undesirable impact of upward forces on the production of gait and balance.

Together, these observations reveal that the prevailing utilization and design of clinical bodyweight support systems are suboptimal for the rehabilitation of posture and gait. The abnormal patterns of muscle activity resulting from high levels of bodyweight support suggest that current practices may even be detrimental for relearning to walk. On the contrary, the gravity-assist established a rehabilitation environment that is mechanically and physiologically optimized for user-specific needs—the external constraints are adapted to the residual motor control abilities of each user. The resulting facilitation of gait execution was unexpected. Subjects with SCI or stroke immediately exhibited improved locomotor performance, which translated into the ability to walk overground for non-ambulatory individuals with sufficient residual control over leg muscle activity. For the less affected individuals, the gravity-assisted enabled the execution of skilled locomotor activities that were not possible without robotic assistance. This result is important since task-specific training determines the outcome of gait rehabilitation after neurological disorders (16).

The gravity-assist augmented the oscillations of the center of mass, which improved the energetic exchanges between kinetic energy and potential energy. Both healthy and neurologically impaired subjects improved the efficacy of their inverted pendulum-like gait movements despite the application of a bodyweight support against the direction of gravity. Beyond the importance of these energetic exchanges for gait efficacy (1, 5, 6), we surmise that gravity-dependent gait interactions are critical for learning and relearning to walk. For example, evidence suggests that gravity-dependent gait interactions during the first unsupported steps in toddlers act as a functional trigger for gait maturation (28, 31), even though this cannot be compared with a situation of a user of a support for the rehabilitation of the locomotor system, wherein said user is suffering of neurological disorder.

The short-term effects of the gait training session with gravity-assist corroborated this hypothesis. It was found that a regular overground walking session with gravity-assist improved the locomotor performance of subjects with SCI. The improvements persisted during overground locomotion without robotic assistance. A similar learning did not occur during gait training on the treadmill with trunk assistance restricted to the upward direction. Instead, the gravity-assist encouraged the subjects to re-use their residual motor control abilities in order to re-master the interactions between body mechanics and gravity. Hence, this type of robotic assistance in a safe yet natural environment represents an ecological approach to gait rehabilitation (32).

The significant role of gravity-dependent gait interactions in enabling and improving motor control after neurological disorders stresses the importance of optimizing the hardware and software underlying the gravity-assist algorithm. In the present scenario, the forces applied to the trunk remained constant throughout gait execution. Moreover, the gravity-assist only targeted upward and forward oscillations. However, an inverted pendulum-like gait behavior requires precisely timed trunk movements in multiple directions (4, 17, 19, 28). For example, the cyclic mediolateral movements of the trunk play a pivotal function in the maintenance of dynamic balance during locomotion.

There is overwhelming evidence in animal models (23, 33-37) and human users (25, 35, 38-42) that electrical stimulation of the spinal cord facilitates leg motor activity after SCI. For example, epidural electrical stimulation of lumbar segments enabled individuals with a functionally complete SCI to exert a voluntary control over the amplitude of leg muscle activity during manually-assisted stepping on a treadmill (25).

In the present study, the opportunity was given to deliver electrical stimulation of the spinal cord to facilitate gait rehabilitation with gravity-assist in one individual with a chronic SCI. The participant exhibited pronounced motor deficits on the left side, which prevented her from performing adequate movements of the left leg during gait rehabilitation. Walking was restricted to a limited number of steps with high levels of bodyweight support. Consequently, we configured the locations and parameters of stimulation in order to facilitate motor control of the left leg. For this, the conceptual framework previously established in rodent models was exploited (29, 30, 36).

Specifically, proprioceptive feedback circuits associated with the modulation of leg muscles were targeted that were minimally active during locomotion. This targeted stimulation enhanced the descending motor control of these muscles, which improved a number of relevant kinematic features during gait rehabilitation. Contrary to severely injured rodents that only exhibited locomotor improvement with robotic assistance and electrochemical stimulation, the less affected participant regained the ability to walk overground using a walking aid but without gravity-assist and without stimulation. Moreover, these improvements persisted more than ten months after the end of the gait rehabilitation program. Previous studies also reported improvements of motor performance in response to rehabilitation facilitated by electrical stimulation of the spinal cord in individuals with functionally complete (25) and incomplete SCI (38, 40).

There is evidence suggesting that gait rehabilitation should be conducted overground (43), across multiple activities of daily living (16, 44), with adequate support conditions (43, 45, 46), motor control enabling systems (45, 47-50), unconstrained arm movements.

The following examples further illustrate the application

### Material and Methods

Study Design

A gravity-assist algorithm was developed that automatically adjusts the forces applied to the trunk based on user-specific needs, and demonstrated the ability of this gravity-assist algorithm to mediate short-term and long-term improvements of locomotor performance in users with SCI and stroke. For this purpose, 8 experimental protocols were implemented that were approved by the local ethical committee of the Canton de Vaud (Switzerland, n° 141/14). The evaluations were conducted at the University Hospital of Vaud, Lausanne, Switzerland (CHUV).

Experimental protocol 1: The properties of the neurorobotic platform was validated during locomotion along straight and curvilinear paths in 8 healthy individuals.

Experimental protocol 2: The impact of upward and forward forces applied to the trunk on the kinematics, kinetics and muscle activity underlying quiet standing and locomotion was characterized. These evaluations have been conducted in a group of 5 healthy individuals.

Experimental protocol 3: To develop an algorithm that automatically tailors the upward force for user-specific needs, experimental recordings were conducted during quiet standing and locomotion over a broad range of upward forces in a total of 9 subjects with a SCI or a stroke.

Experimental Protocol 4: To develop a decision map that automatically adjusts the forward force correction based on the walking speed and user-specific needs, both computational simulations using a passive walker and experimental recordings in a cohort of 28 subjects with a SCI or a stroke were conducted.

Experimental Protocol 5: To validate the gravity-assist algorithm, the upward and forward forces were configured based on the algorithms in 6 subjects with a SCI or a stroke. These subjects were evaluated during locomotion with gravity-assist and with small variations of the upward and forward forces.

Experimental Protocol 6: The, the ability of the gravity-assist was evaluated to improve locomotor performance during natural walking and skilled locomotion along the irregularly spaced rungs of a horizontal ladder. These evaluations were performed in a total of 13 subjects with SCI and 13 subjects with stroke. Locomotor performance were quantified based on the difference with kinematic features recorded in a group of 13 healthy individuals.

Experimental Protocol 7: To provide insights into the therapeutic potential of the gravity-assist for gait rehabilitation, the short-term effect of a single training session performed on a treadmill with body-weight support compared to overground with gravity-assist was studied. These evaluations were conducted one week apart on the same 5 subjects with a SCI.

Experimental Protocol 8: A feasibility study was conducted to evaluate the long-term effects of gait rehabilitation with gravity-assist. For this purpose, a prospective evaluation of locomotor performance was performed in a non-ambulatory subject with a chronic SCI (AIS-C) who was trained for 8 months with gravity-assist. To enable motor control during training, epidural electrical stimulation of the lumbar spinal cord was delivered using an electrode array that was originally implanted to alleviate neuropathic pain. The parameters of the gravity-assist and stimulation were updated weekly.

All measurements were obtained using objective readouts with high-precision equipment. Blinding during data acquisition and analysis was not possible because of the obvious differences between the experimental conditions (e.g. with and without robot). All the recorded gait cycles were included in the analyses. No statistical outliners were excluded.

### Participants

A total of 26 subjects with a SCI or a stroke and 13 healthy individuals took part in the study. Written informed consent was obtained for each participant. The experimental protocols conformed to the latest revision of the Declaration of Helsinki. All the subjects with neurological deficits were followed by a physician from the neurorehabilitation department (S.C). Prior to their enrollment in the study, the medical history of the subjects was collected together with standard neurological evaluations. Motor scores were measured using the Motricity Index (54) while the severity was evaluated using the Functional Independence Measure (FIM) (55). ASIA scores and WISCI-II was also collected in subjects with SCI. The main characteristics of each neurologically impaired subject enrolled in the experimental protocols are summarized in the following Table.

Spinal Cord Injury (SCI)

| | AIS | Age | Gender | Weight | Time after injury | Lesion | |
|---|---|---|---|---|---|---|---|
| Subject ID | grade | (years) | (M/F) | (kg) | (months) | level | Origin of the injury |
| SCI_HCU | D | 37 | M | 82 | 182 | C8 | Traumatic (sport) |
| SCI_CFR | D | 64 | F | 50 | 180 | T8/T10 | Peridura anaesthesia issue |
| SCI_DMZ | C | 27 | M | 63 | 36 | C7/D1 | Traumatic (sport) |
| SCI_GBA | D | 38 | M | 82 | 264 | T5 | Spondylodiscitis |
| SCI_FIM | D | 62 | F | 55 | 156 | D10 | Peridura anaesthesia issue |
| SCI_BTA | D | 53 | M | 86 | 21 | T6 | Ischemia (scuba diving) |
| SCI_LST | D | 30 | M | 75 | 22 | C4 | Ischemia (scuba diving) |
| SCI_MRC | D | 62 | F | 68 | 12 | T1 | Herniated disc |
| SCI_KGY | D | 40 | M | 68 | 248 | T6 | Spondylodiscitis |
| SCI_POR | D | 58 | M | 90 | 39 | C7 | Traumatic (sport) |
| SCI_EDE | C | 59 | M | 76 | 14 | D11 | Herniated disc |
| SCI_BME | C | 62 | F | 54 | 16 | T10 | Ischemia |
| SCI_TFA | C | 24 | M | 85 | 18 | T7 | Traumatic (car accident) |

Stroke (STK)

| | Motor | Age | Gender | Weight | Time after stroke | Severity | |
|---|---|---|---|---|---|---|---|
| Subject id | score | (Years) | (M/F) | (kg) | (months) | index | Origin of the stroke |
| STK_CZI | 54 | 66 | F | 66 | 235 | 103 | Ischemia |
| STK_FDJ | 200 | 39 | M | 99 | 14 | 116 | Ischemia |
| STK_GTO | 64 | 53 | F | 62 | 10 | 95 | Haemorrage |
| STK_JEI | 84 | 43 | M | 53 | 12 | 96 | Ischemia |
| STK_LCS | 152 | 32 | M | 64 | 10 | 107 | Haemorrage |
| STK_PMA | 140 | 64 | M | 88 | 60 | 110 | Ischemia |
| STK_RSC | 152 | 68 | M | 78 | 12 | 68 | Haemorrage |
| STK_TMO | 20 | 62 | F | 49 | 8 | 41 | Haemorrage |
| STK_SBE | 152 | 36 | F | 96 | 20 | 126 | Ischemia |
| STK_CPE | 152 | 52 | F | 69 | 50 | 116 | Haemorrage |
| STK_CFO | 165 | 36 | M | 77 | 108 | 126 | Ischemia |
| STK_CAR | 25 | 62 | M | 64 | 8 | 32 | Ischemia |
| STK_JPA | 144 | 58 | M | 90 | 120 | 118 | Ischemia |

### Neurorobotic Platform

To enable kinematics, kinetics and muscle activity recordings during standing and walking, commercially available technologies were integrated.

Previously a multidirectional robotic interface was developed for subjects that provides adjustable trunk support in each of the Cartesian directions and in rotation (20). To further develop a similar body-weight support system for humans, cable robot technology (56) was exploited. An overhead body-weight support system was designed that precisely controls the forces applied to the trunk in each of the Cartesian directions. The technical features of the robot have been described previously (56). Briefly, two parallel rails are arranged horizontally on the ceiling and tilted by 45 deg towards the workspace along their longitudinal axis. The rails are located at 3.5 m from the ground and cover a footprint of 11.5 m by 2.5 m. Each rail guides two deflection units composed of a ball-beared cart carrying an inclinable pulley. The inclination axis of the pulley is parallel to the rail. A Dyneema cable connects the two carts on one rail in order to form trolleys. Motorized winches actuating the Dyneema cables are positioned at the extremities of the rails. Four elastic elements consisting of spiral steel springs each with a parallel rubber cord inside connect the cables to stainless steel rings. The arrangement allows the four cables to intersect at a specific point, termed the node. Winch positions are measured by encoders on the motor shafts, while the length of the elastic elements is monitored using wire potentiometers. An inertial measurement unit (IMU) combining accelerometers, gyroscopes and a magnetometer are located in the node. These sensors provide redundant information allowing to calculate the position of the node and resultant force vector on the subject through optimization. Control algorithms have been detailed previously (56). Communication procedures are implemented in Matlab using an EtherCat network operating at 1 kHz (57).

The subjects were attached to the robot using a commercially available harness (Maine Anti-Gravity Systems, Inc., USA). The two shoulder straps of the harness are attached to the two outer ends of a plate by means of buckles. The plate itself is pivot-mounted to the lower end of the node.

The plate can rotate infinitely, allowing the subject to take arbitrary turns. The robot enabled subjects to walk freely within a 20 m² workspace (10 m length by 2 m width by 2.6 m height). The robot is capable of supporting 100 kg, with a maximal upward support of 90 kg and a maximal forward force of +/−5 kg in the lateral and longitudinal directions. A fall detector and smooth counteraction mechanism guaranteed user safety in case of a fall.

These technologies were integrated within a neurorobotic platform that combines (i) a physiological recording unit monitoring kinematics, kinetics and muscle activity signals, (ii) a robotic body-weight support system (20) and (iii) a control processing unit. The control processing unit allowed real-time tuning of robotic actuation, updates of an augmented reality environment and modulation of neuroprostheses based on any of the recorded signals. All three units were interconnected via an Ethernet network using a real-time with EtherCat bus, as previously described for the design of the rodent neurorobotic platform (26).

### Behavioral Tasks and Experimental Protocols

The subjects were recorded during standing or walking without or with robotic assistance across four behavioral paradigms: quite standing onto the force-plates, locomotion along a straight path, locomotion along a sinusoidal path projected onto the floor, walking along a real or projected horizontal ladder with irregularly positioned rungs.

### Data Acquisition and Analysis

Standardized procedures to record kinematics, kinetics, and muscle activity, as published previously (27) were used.

### Kinematic, Kinetic, and Electromyographic Recordings

Kinematic recordings were obtained using a 3D motion capture system (Vicon, UK) featuring fourteen Bonita10 cameras and two Bonita720c DV cameras. Two force plates (9260AA6, Kistler, Switzerland) were positioned within the ground in the middle of the workspace to monitor ground reaction forces and center of foot pressure displacements. Electromyographic activity was monitored using a 16-channel wireless recording system (Myon 320, Myon AG, Switzerland). These technologies were integrated within the neurorobotic platform according to published methods (26).

Trunk, head and bilateral leg and arm kinematics were recorded using 34 markers positioned overlying anatomical landmarks defined by the full-body Plug-In-Gait model developed by Vicon. The 14 cameras covered a 12×4×2 m workspace. The movement of assistive devices was monitored using reflective markers. Video recordings were obtained at 100 Hz. 3D position of the markers was reconstructed offline using Vicon Nexus software. The body was modeled as an interconnected chain of rigid segments. Anthropometric data (body height, body weight, widths of the joints) were added to the full-body Plug-In-Gait model to determine the positions of joint centers, and calculate the elevation and joint angles of the lower limbs. The ground reaction vector and antero-posterior and medio-lateral torques were acquired using two force plates integrated in the floor. Bipolar surface electrodes (1 cm diameter, electrode separation of 1 cm) were placed over the following leg muscles to record electromyographic activity: soleus, medial and lateral gastrocnemius, tibialis anterior, semitendinosus, biceps femoris, vastus lateralis and rectus femoris. During standing and walking, kinetic and electromyographic signals were sampled at 1 kHz, amplified, synchronized on-line with kinematic data, and stored for off-line analysis. Electromyographic signals were filtered offline (band-pass 10-450 Hz). Electromyographic recordings were sampled at 2 kHz during electrophysiological evaluations. During these recordings, the torques developed at the ankle and knee joint levels were measured (1 kHz) using an isokinetic dynamometer chair (Humac Norm, Computer Sports Medicine, USA).

For locomotion, a total of 140 parameters were computed from kinematic, kinetic and muscle activity recordings according to published methods (20, 27, 30, 36). Parameters were calculated semi-automatically using customized code implemented in Matlab. Clinical gait reports were reported automatically for each set of trials. The clinical gait reports aimed to provide to the medical doctors and their subjects, pleasant-to-read documents that allow quick visualization of the origins and the magnitude of the gait deficits and their adjustments when using the gravity-assist. For standing, we computed a total of 15 parameters that are typically used to evaluate postural control. To quantify the effects of experimental conditions on the control of standing and walking and measure locomotor performance objectively, we applied a principal component (PC) analysis (20, 30, 36).

### Principal Component Analysis

PC analysis was applied to parameters computed from recordings obtained during quiet standing and locomotion. PC analyses were applied using the correlation matrix (20, 30, 36). Three types of datasets were examined with a PC analysis. For quiet standing, the PC analysis was applied on a set of 15 kinematic, kinetic and electromyographic parameters computed on 40 time-windows lasting 1 second for each experimental condition per subject. The analysis was applied for each subject independently. For locomotion, the PC analysis was applied to all the computed kinematic parameters from all individual gait cycles from all the subjects simultaneously, or on all the computed kinematic and muscle activity parameters for each subject independently. Galt cycles and postural time-windows were visualized in the new synthetic space defined by the two first PCs. The performance was measured as the Euclidian distance between the data points in PC1-PC2 space and the mean position of data points obtained in healthy individuals (20, 30, 36). The relevant parameters to account for differences between experimental conditions or subjects were extracted based on the factor loadings (correlation) of individual parameters onto each PC.

### Statistical Analysis

All data are reported as mean values±SEM, unless specified otherwise. The non-parametric Mann Whitney U test and the non-parametric Kruskal-Wallis test were used when comparing gait cycles. Two-ways ANOVA was used to compare subjects and conditions. Paired Student t-test was used for repeated measures when data were distributed normally, otherwise the Wilcoxon signed-rank test was used instead. Anderson-Darling test was used to evaluate normal distribution. Post-hoc comparisons were performed using the Tukey-Kramer test when appropriate. Statistical tests are specified in the legends of figures.

### Example 1

### Experimental Protocol 1: Properties and Validation of the Neurorobotic Platform

Eight healthy subjects were recorded during locomotion without and with robot along a straight or curvilinear path projected on the floor using the augmented reality system. They were asked to walk naturally at their own selected pace. They wore the harness during both conditions. The robot was configured in transparent mode, which corresponds to the minimal upward force (4 kg) necessary to enable robot-subject interactions. For each condition, a total of 10 steady-state gait cycles were recorded and analyzed.

### Example 2

### Experimental Protocol 2: Impact of Upward and Forward Forces on Posture and Gait

Five subjects were first recorded during quiet standing while attached to the robotic interface. They were asked to stand quietly with eyes open. Each foot was positioned on its own force plate with a standardized location and orientation (58). The distance between the medial side of the heels was set at 8.4 cm and the external rotation angle of the feet was kept at 9 deg with respect to the sagittal plane. The subjects were instructed to center and stabilize their center of foot pressure that was projected on the floor in real-time through the augmented reality system. This visual biofeedback was removed during recordings. The subjects were then asked to stare at a visual reference mark projected on the floor 3 m straight ahead in front of them. Each trial lasted 20 seconds. Two trials were collected for each upward and forward force.

The same subjects were then evaluated during locomotion across a broad range of upward and forward forces. The subjects were asked to walk naturally at their own selected pace. For each condition, a total of 10 steady-state gait cycles were recorded and analyzed.

### Example 3

### Experimental Protocol 3: Design of Gravity-Assist Algorithm: Personalization of Upward Force

Nine subjects with a SCI or a stroke were tested during quiet standing over the maximum possible range of upward forces under the same conditions as explained in Experimental Protocol 2. For each upward force, two trials were collected that each lasted 20 s and were separated by 1 min. Each trial was then divided into a set of 20 windows of 1 s over which 15 kinematic, kinetic and electromyographic parameters were computed. A PC analysis was applied on these variables to determine the optimal upward force for each subject.

The this dataset and results were used to build an artificial neural network that calculated the necessary correction of upward force to provide each subject with optimal upward force. The artificial neural network integrated the kinematic and kinetic parameters (n=12, 10 seconds of recording). 13 learning rules and 11 structures were tested with different numbers of neurons in the hidden layer. Cross-validation (20% of data were randomly picked for validation) was performed to select the one-layer feedforward model with log-sigmoid hidden neurons and linear output neurons showing the lowest mean squared error (Neural Network Toolbox from Matlab). The selected model combined 9 neurons and learned rules through the Levenberg-Marquardt algorithm. The the selected model was fed with a test dataset in order to validate the properties of the artificial neuronal network.

### Example 4

### Experimental Protocol 4: Design of Gravity-Assist Algorithm: Personalization of Forward Force

To calibrate the forward force correction, we conducted computational simulations using a passive walker model composed of a point mass m=80 kg and two weightless segments attached to this mass, which represent the legs. A linear spring element of stiffness k was inserted in the leg model in order to enable deformations similar to a spring-loaded inverted pendulum (59). The model is subjected to a force parallel to the walking direction. Consequently, damping elements of damping constant d were added in the legs in order to enable a limit-cycle behavior of the passive walker. The angle of the leg at the end of swing was defined as α. During the swing phase, the length of the leg equaled the resting length l₀. Consequently, the occurrence of the foot strike was deduced from simple geometrical calculations. The end of the stance phase was defined at the moment when the length of the leg increased beyond l₀. Forward and upward forces were added to emulate the robotic assistance. The behavior of the passive walker model was determined by the following equation:

$${\vec{F}}_{tot} = - ( ‖ \vec{u} ‖ - {l}_{0} ) \cdot k \cdot \frac{\vec{u}}{‖ \vec{u} ‖} - m \cdot g \cdot \vec{{e}_{y}} - d \cdot \frac{\vec{\dot{u}} \cdot \vec{u}}{{{‖}^{\to } ‖}^{2}} \cdot \vec{u} + {F}_{upward} \cdot \vec{{e}_{y}} + {F}_{forward} \cdot \vec{{e}_{x}}$$

$$\vec{u} = [ \begin{matrix}x \\ y\end{matrix} ]$$

with x and y describing the position of the center of mass, Fₜₒₜ the total force applied to the model, F_{upward} the upward force applied to the model, F_{forward} the forward force applied to the model, and e_{x,y} the direction vector.

The model was tested under different conditions of upward and forward forces and for each pair of upward-forward values. For each pair, we performed simulations with all possible combinations of α, l₀, and k values that fit within the range of plausible locomotor behaviors. The values that yielded the closest step length sl, gait speed gs and gravity-dependent energetic exchanges ΔEpot compared to locomotion without the added forward and upward forces were selected. The recovery of gait parameters with forward force corrections was calculated as follows:

$$R = 1 - ( \begin{matrix}\begin{matrix}\frac{\vert {sl}_{{Upward}_{x} , {Forward}_{y}} - {sl}_{no} \vert}{\vert {sl}_{{Upward}_{x} , {Forward}_{0}} - {sl}_{no} \vert} + \\ \frac{\vert {ws}_{{Upward}_{x} , {Forward}_{y}} - {ws}_{no} \vert}{\vert {ws}_{{Upward}_{x} , {Forward}_{0}} - {ws}_{no} \vert} +\end{matrix} \\ \frac{\vert Δ {pot}_{{Upward}_{x} , {Forward}_{y}} - Δ {Epot}_{no} \vert}{\vert Δ {Epot}_{{Upward}_{x} , {Forward}_{0}} - Δ {Epot}_{no} \vert}\end{matrix} ) / 3$$

The a total of 26 subjects with a SCI or a stroke were recorded. The subjects were recorded during locomotion with the upward forces predicted by the artificial neural network, and a narrow range of forward forces centered around the optimal values predicted by the simulations. The subjects were asked to walk at their own, comfortable pace. For each condition, the subjects performed 3 or 4 trials during which they walked straight ahead over a distance of approximately 10 m.

The gait parameters were then represented in a three-dimensional space whereby x-axis is the amount of upward force, y-axis is the walking speed v normalized by leg length l (Froude number: v²/(g·l)), z-axis is the amount of forward force. We then fit a polynomial function to the data. For this, we tested 25 two-dimensional polynomial functions with degrees on x- and y-axes ranging from 1 to 5. For each polynomial, 75% of the data were randomly selected. A polynomial function was fitted to this dataset. The accuracy was evaluated using a 500-fold cross-validation and root mean square error (RMSE) on the 25% remaining data. The selected polynomial function was applied on the entire dataset to generate a final decision map.

### Example 5

Experimental Protocol 5: Validation of the Gravity-Assist Algorithm to Enable Locomotion in Individuals with SCI or Stroke

Six subjects with a SCI or stroke were recorded during quiet standing with the optimal upward force calculated by the artificial neural networks. Two trials lasting 20 s each were recorded. Recordings were then performed with the addition and subtraction of an upward force corresponding to 10% of the bodyweight, resulting in 3 upward force conditions. Two trials were acquired per condition. The decision map was used to define the forward force correction for these three upward forces, and the subjects were then recorded during locomotion with these configurations. The subjects were asked to walk at their own, comfortable pace. For each condition, the subjects performed 3 or 4 trials during which they walk straight ahead over a distance of approximately 10 m. The two first steps (gait initiation) and the two last steps (gait termination) were rejected from the analysis.

### Example 6

Experimental Protocol 6: The Gravity-Assist Improves Locomotor Performance in Individuals with SCI and Stroke

Twenty-six subjects with a SCI or stroke were recorded during quiet standing in order to configure the gravity-assist. Ambulatory subjects were then asked to walk in the straight direction at their own, comfortable pace using their preferred assistive device (without robot). All the subjects were then recorded during locomotion with gravity-assist. A total of 15.9+/−0.85 gait cycles were analyzed for each subject and condition. Subjects with sufficient locomotor performance were finally tested during skilled locomotion along a horizontal ladder consisting of a succession of ten irregularly spaced rungs (10 cm width) with gaps of 0, 10, 20 or 30 cm. The total length of the ladder was 270 cm. The ladder was located 15 cm above the ground. To decide whether the subjects could be tested in this task, a simple rule was elaborated based on the feeling of the subjects and the advice from the physical therapist. Without the gravity-assist, the vast majority of the subjects could not perform the task. In order to obtain a baseline, these subjects were asked to walk along the same ladder layout that was projected on the floor by means of the augmented reality system. For each subject and condition, the walking speed and the precision of foot placement were evaluated.

### Example 7

Experimental Protocol 7: The Gravity-Assist Improved Locomotor Performance after One Gait Training Session

Five subjects with a spinal cord injury participated in two training sessions, separated by one week (FIG. 7A). During the first session (60 min), subjects walked overground with gravity-assist. During the second session (week 2), they were asked to walk the same distance on a treadmill with the same upward force, but without forward force corrections. Immediately before and after each training session, the subjects were recorded during overground locomotion without gravity-assist at their own selected pace. They were allowed to use their preferred assistive device. During each training session, subjects were allowed to rest when necessary. A few days later, all the participants were asked to fill a survey that aimed to determine their perceived differences between both paradigms. Most of the participants expressed their satisfaction on the following points: 1) that they felt a more important breathing fatigue after the training session on the treadmill compared to overground; 2) that they felt a more important muscular fatigue after the training session on the treadmill compared to overground; 3) that during the training session performed overground with the robotic assistance, they felt that their gait pattern was more natural and less constraining compared to the training session on a treadmill; 4) that at the end of the training session performed overground with the robotic assistance, while walking overground without the robotic assistance, they felt that their gait pattern was more natural and less constraining compared to the training session on a treadmill; 5) that a gait rehabilitation program performed overground with the robotic assistance, would be more beneficial than a gait rehabilitation program performed on a treadmill in order to improve locomotor performance; 6) that a training session performed overground with the robotic assistance, provided more satisfaction than a training session performed on a treadmill.

### Example 8

Experimental Protocol 8: Training with Gravity-Assist and Electrical Spinal Cord Stimulation Promotes Durable Recovery

A user SCI_MRC was enrolled in a multi-pronged gait rehabilitation program including overground locomotor training with gravity-assist and electrical stimulation of the lumbar spinal cord. This user was 62 years old at the time of the inclusion in the study and had suffered a herniated disc at C6/C7 level 12 months before the inclusion in the study (FIG. 8A). She followed a conventional gait rehabilitation program during one year, including 8.5 months in the Swiss Paraplegic Center (SUVA Sion, Switzerland). At this stage, the subject had recovered sensitivity below the injury and minimal motor control in the right leg. However, the nearly complete lack of motor control on the left leg bound her to a wheelchair. She had been surgically implanted with an electrode array (Specific 5-6-5, Medtronic, USA) located epidurally over lumbar spinal cord segments in order to alleviate pain (FIG. 8C). The array was connected to an implantable pulse generator (Activa RC, Medtronic, USA).

The participant followed a gait rehabilitation program that took place 2 to 3 times per week for 8 months. A typical gait training session started on a treadmill with manual assistance as needed and epidural electrical stimulation of lumbar spinal segments. This first component of the session lasted about 15 min. The participant then walked overground with gravity-assist and epidural electrical stimulation of lumbar spinal segments for approximatively 30 min (FIG. 8C). The remainder of the session was used for additional exercises and stretching. Once the subject was able to walk overground with a walker, the program gradually transitioned to include walking with the walker without gravity-assist, without electrical stimulation and at home. The gravity-assist and spinal cord stimulation features were adjusted within each session and over time according to user-specific needs. Throughout the course of the gait rehabilitation program, the stimulation amplitude and amount of upward force was systematically decreased in order to promote the recovery of walking without robot and without stimulation. Evaluations with kinematic and muscle activity recordings were performed and analyzed for various time points that are described in FIG. 8D.

### Maximal Voluntary Contraction

The participant sat on the chair of the isokinetic dynamometer with the hip angle fixed at 80 deg of flexion (0 deg=standing extension). The trunk was stabilized with two crossed safety belts. Recordings were performed while the knee and ankle joints were fixed at 90 deg of flexion. The anatomical flexion-extension axis of these joints were aligned with the rotation axis of the device. For each condition (left or right, knee or ankle), the participant was instructed to gradually increase her force from rest to maximum capacity. The produced torque was displayed in real time to provide the participant with a feedback about her performance and to motivate her to deliver a true maximal effort. The task was repeated four times per condition, with 1 min rest between each attempt. The maximal voluntary contraction value was calculated over a 500 ms time-window around the peak torque and averaged across the four attempts.

### Functional Mapping of the Spinal Cord

During functional mapping experiments, the user was asked to lay on a bed in supine position with the legs extended. Motor responses were recorded from all the selected muscles (see section Kinematic, kinetic, electromyographic recordings) while delivering rectangular bi-phasic pulses through the implanted epidural electrode array (0.15 ms duration at 2 Hz) through different electrode configurations. The intensity of the electrical stimulation was increased from 0 to 3 V and each intensity was repeated up to 30 times for statistical significance. Electromyographic signals were filtered (10 to 800 Hz bandpass) and rectified. The maximal amplitude of the motor responses was calculated for each muscle and averaged over all the repetitions. The resulting recruitment curves were then normalized to the maximum values obtained for each muscle across all the tested electrode configurations and stimulation amplitudes. To visualize the spatial distribution of motoneuron activation in the spinal cord, electromyographic signals were projected onto the location of motoneuron columns according to published methods (30).

### Epidural Electrical Stimulation of the Lumbar Spinal Cord

The spatial distribution of motoneuron activation in response to stimulation through each of the electrodes of the array was used to configure the stimulation protocols. Specifically, three electrode configurations were selected that targeted the entire left side of the lumbar spinal cord (FIG. 8). Electrode 3 vs case (anode), providing activation of the most rostral segments, electrode 5 vs case (anode), providing activation of the central spinal cord and a tri-polar combination of electrode 15 (cathode) versus 14 and 13 (anodes) providing activation of the most sacral segments. The stimulation pulses were interleaved by 2.5 ms and delivered at 40 Hz.

In connection with the above disclosure especially the following aspects are explicitly disclosed:

Aspect 1: Apparatus comprising a support system for a user, said apparatus comprising a controller for said support system, said controller comprising:
a. means for applying one or more of z-direction force F_{zsup}, x-direction force Fₓₛᵤₚ and y-direction force F_{ysup}, or any combination thereof on said user according to the following respective equations:

F_{zsup}=Fz(x,dx/dt,y,dy/dt,z,dz/dt);

Fₓₛᵤₚ=Fx(x,dx/dt,y,dy/dt,z,dz/dt);

F_{ysup}=Fy(x,dx/dt,y,dy/dt,z,dz/dt);
wherein
   Fₓₛᵤₚ is the force applied in forward direction,
   F_{ysup} is the force applied in lateral direction and
   F_{zsup} is the force applied in upward direction;
   x, y, and z are the forward, lateral, and vertical coordinate positions of the center of mass in a coordinate system that is fixed to the stance foot and rotates with the person, and dx/dt, dy/dt, dz/dt are the derivatives with respect to time.
b. optionally means for applying further forces on said user.

Aspect 2: Apparatus according to aspect 1, wherein said means apply said upward force F_{zsup} according to the following equation:

F_{zsup}=c_{z}·(z₀−z)+Δm·g,
wherein
c_{z} is the stiffness, which is chosen such that said user walks with a frequency of natural walking;
z is the vertical position of the center of mass of said user;
z₀ is the average or nominal walking height;
Δm is the part of the mass of said user that is compensated by said upward force;
g is gravity acceleration;
Fₓₛᵤₚ and F_{ysup} are nul.

Aspect 3: Apparatus according to aspect 1, wherein said means apply said forward force Fₓₛᵤₚ according to the following equation:

Fₓₛᵤₚ=cₓₛ·sin(a_{z}·dz/dt) for z≤z₀,

Fₓₛᵤₚ=0 for z>z₀,
wherein
a_{z} and cₓₛ are positive constants,
z₀ is the average or nominal walking height;
or according to the following equation:

Fₓₛᵤₚ=−cₓ·x;
wherein cx is a positive constant
or according to the following functions:

Fₓₛᵤₚ=Fₓₛᵤₚ(z,dz/dt)

or

Fₓₛᵤₚ=Fₓₛᵤₚ(x)

Aspect 4: Apparatus according to aspect 1, wherein said means apply said lateral force F_{ysup} according to the following equation:

F_{ysup}=c_{y}·y;
wherein c_{y} is stiffness.

Aspect 5: Apparatus according to any one of aspects 1-4, wherein said controller is passive.

Aspect 6: Apparatus according to aspect 5, wherein said means apply said upward force according to the following equation:

F_{zsup}=F_{zsup}(Fₓₛᵤₚ,dx/dt,dz/dt);
whereby F_{zsup} suffices the following inequality constraint:

F_{zsup}<−Fₓₛᵤₚ(dx/dt)/(dz/dt);
or said forward force according to the following equation:

$${F}_{xsup} = \begin{matrix}\{ {F}_{xsup} ( z , dz / dt ) & for dz / dt < 0 and z < {z}_{0}\end{matrix}$$

$$\begin{matrix}\{ {F}_{xsup} ( x ) & for dz / dt > 0 and z < {z}_{0}\end{matrix}$$

$$\begin{matrix}\{ 0 & for z > {z}_{0}\end{matrix}$$

Aspect 7: Apparatus according to any one of aspects 1-6, further comprising means for measuring the shift of the mean antero-posterior position of the center of plantar pressure of said user and means for applying forward force to said user in order to compensate said shift.

Aspect 8: Apparatus according to any one of aspects 1-7, further comprising:
c. means for setting the apparatus in transparent mode;
d. means for computing parameters from kinematic recordings of locomotor tasks performed by said user to obtain and optionally storing a dataset;
e. means for elaborating said dataset with principal component (PC) analysis.

Aspect 9: Apparatus according to aspect 7, wherein said means for measuring the shift of the mean antero-posterior position of the center of plantar pressure of said user and means for applying forward force to said user in order to compensate said shift use an artificial neural network.

Aspect 10: Apparatus according to any one of aspects 1-9, wherein said apparatus is provided with a recording platform for real-time acquisition of apparatus-user interactions.

Aspect 11: Apparatus according to any one of aspects 1-10, wherein said apparatus is selected from the group consisting of cable robot, trunk support, exoskeleton, wearable exoskeleton and exosuit.

Aspect 12: Apparatus according to any one of aspects 1-11, also comprising a device for epidural or subdural electrical stimulation.

Aspect 13: Apparatus of any one of aspects 1-12 for use in restoring voluntary control in a user.

Aspect 14: Apparatus for use according to aspect 13, wherein said user is suffering from a neuromotor impairment.

Aspect 15: Apparatus for use according to aspect 14, wherein said neuromotor impairment is selected from the group consisting of partial or total paralysis of limbs.

Aspect 16: Apparatus for use according to aspect 14 or 15, wherein said neuromotor impairment is consequent to a spinal cord injury, an ischemic injury resulting from a stroke, a neurodegenerative disease, Amyotrophic Lateral Sclerosis (ALS) or Multiple Sclerosis.

Aspect 17: Apparatus for use according to any one of aspects 13-16, coupled with a device for epidural or subdural electrical stimulation.

Aspect 18: A method for operating the apparatus of any one of aspects 1-12, in particular for control of locomotion, wherein a user is connected to said apparatus, comprising the following steps:
a. setting the apparatus to apply one or more of z-direction force F_{zsup}, x-direction force Fₓₛᵤₚ and y-direction force F_{ysup}, or any combination thereof on said user according to the following respective equations:

F_{zsup}=Fz(x,dx/dt,y,dy/dt,z,dz/dt);

Fₓₛᵤₚ=Fx(x,dx/dt,y,dy/dt,z,dz/dt);

F_{ysup}=Fy(x,dx/dt,y,dy/dt,z,dz/dt);
wherein
Fₓₛᵤₚ is the force applied in forward direction,
F_{ysup} is the force applied in lateral direction and
F_{zsup} is the force applied in upward direction;
b. optionally applying further forces on said user.

Aspect 19: Method according to aspect 18, wherein said upward force F_{zsup} is applied according to the following equation:

F_{zsup}=c_{z}(z₀−z)+Δm·g,
wherein all the definitions are provided as above.
Fₓₛᵤₚ and F_{ysup} are nul.

Aspect 20: Method according to aspect 18, wherein said forward force Fₓₛᵤₚ is applied according to the following equation:

Fₓₛᵤₚ=cₓₛ·sin(a_{z}·dz/dt) for z<z₀,

Fₓₛᵤₚ=0 for z>z₀
wherein all the definitions are provided as above;
or according to the following equations:

Fₓₛᵤₚ=Fₓₛᵤₚ(z,dz/dt)

Or

Fₓₛᵤₚ=Fₓₛᵤₚ(x).

Aspect 21: Method according to aspect 18, wherein said lateral force F_{ysup} is applied according to the following equation:

F_{ysup}=c_{y}·y.
wherein all the definitions are provided as above.

Aspect 22: Method according to any one of aspects 18-21, comprising the following steps:
a. setting the apparatus to apply an upward force on said subject in quiet standing;
b. measuring the shift of the mean antero-posterior position of the center of plantar pressure of said subject of the postural maintenance of said subject;
c. setting the apparatus to apply a forward force to said subject in order to compensate said shift.

Aspect 23: Method according to any one of aspects 18-21, comprising the following steps:
a. setting the apparatus in transparent mode;
b. having said subject to perform locomotor task;
c. computing parameters from kinematic recordings from said locomotor task to obtain a dataset;
d. submitting said dataset to a principal component (PC) analysis to provide a quantification of locomotor performance of said subject, and extracting parameters accounting for the effects of experimental conditions on locomotor performance of said subject;
e. setting the apparatus to apply an upward force on said subject in quiet standing;
f. measuring the shift of the mean antero-posterior position of the center of plantar pressure of said subject] of the postural maintenance of said subject;
g. setting the apparatus to apply a forward force to said subject in order to compensate said shift.

Aspect 24: Method according to any one of aspects 18-21, comprising the following steps:
a. setting the apparatus in transparent mode, with a first or second subject in standing position, wherein said first subject is a normal subject and said second subject is a subject in need of restoring voluntary control of locomotion;
b. recording whole-body kinematics, ground reaction forces and ankle muscle activity over the maximal possible range of upward forces for said first subject to obtain a first dataset;
c. recording whole-body kinematics, ground reaction forces and ankle muscle activity over the maximal possible range of upward forces for said second subject to obtain a second dataset;
d. applying a Principal Component analysis on said first and second dataset and determining the upward force as the condition with the minimum distance between said second subject and said first healthy subject in the Principal Component space;
e. setting the apparatus to apply an upward force on said second subject in quiet standing;
f. measuring the shift of the mean antero-posterior position of the center of plantar pressure of between said first subject and said second subject;
g. setting the apparatus to apply a forward force to said second subject in order to compensate said shift.

Aspect 25: Method according to aspect 24, wherein step d) is performed using an artificial neural network.

Aspect 26: Method according to aspect 24 or 25, wherein step g) is performed setting said forward force as a function of walking speed of said second subject.

Aspect 27: A computer program for carrying out the method of any one of aspects 18-26.

Aspect 28: A data medium having the computer program of aspect 27.

Aspect 29: A computer system on which the computer program of aspect 27 is loaded.

Aspect 30: Apparatus of any one of claims 1-12 operatively connected to the computer system of aspect 29.

Moreover, the following additional aspects are explicitly disclosed:

Additional Aspect 1: A method for operating a robotic support system comprising:
setting the robotic support system to apply a first force on a subject;
setting the robotic support system to apply a second and/or third force on the subject; and
controlling one or more of the first force, second force, and/or third force in real-time while the subject is performing a rehabilitation training routine.

Additional Aspect 2: The method of additional aspect 1, wherein the robotic support system is a robotic platform that assists body movements.

Additional Aspect 3: The method of additional aspect 1, wherein controlling one or more of the first force, second force and/or third force includes maintaining one or more of the first force, second force, and/or third force substantially constant while the subject is performing the rehabilitation training routine.

Additional Aspect 4: The method of additional aspect 1, wherein controlling one or more of the first force, second force and/or third force includes controlling the first force, second force and/or third force to achieve a desired velocity of the subject performing the rehabilitation training routine.

Additional Aspect 5: The method of additional aspect 1, wherein controlling one or more of the first force, second force and/or third force includes obtaining one or more parameters related to movement of the subject performing the rehabilitation training routine, where the one or more parameters are fed into a model that outputs adjustments to the first force, second force and/or third force.

Additional Aspect 6: The method of additional aspect 5, wherein the model comprises a movement model corresponding to expected or desired movements performed via the subject during the rehabilitation training routine.

Additional Aspect 7: The method of additional aspect 5, wherein the one or more parameters related to movement of the subject include one or more of kinetic activity, kinematic activity, and/or muscle activity from the subject.

Additional Aspect 8: The method of additional aspect 1, further comprising applying neuromodulation to the subject performing the rehabilitation training routine.

Additional Aspect 9: The method of additional aspect 8, wherein neuromodulation includes one or more of electrical stimulation, and/or pharmacological stimulation.

Additional Aspect 10: The method of additional aspect 9, wherein electrical stimulation includes one or more of epidural electrical stimulation, subdural electrical stimulation, and/or functional electrical stimulation, and wherein pharmacological stimulation includes providing at least one agonist of monoaminergic receptors.

Additional Aspect 11: A method for assisting a subject performing a rehabilitation training routine, comprising:
applying one or more of a first force, a second force, and a third force to the subject via a robotic support system to the subject;
monitoring one or more parameters of the subject during the rehabilitation training routine; and
adjusting one or more of the first force, the second force and/or the third force at least in part based on the one or more parameters of the subject.

Additional Aspect 12: The method of additional aspect 11, wherein monitoring one or more parameters further comprises:
monitoring one or more parameters related to movement of the subject, including one or more of kinetic activity, kinematic activity, electromyographic activity and/or measured forces exerted on the subject and forces exerted via the subject on the robotic support system.

Additional Aspect 13: The method of additional aspect 12, wherein adjusting one or more of the first force, the second force and/or the third force includes maintaining a magnitude and/or direction of one or more of the first force, the second force and/or the third force substantially constant for a duration of the rehabilitation training routine based on feedback from the one or more parameters.

Additional Aspect 14: The method of additional aspect 12, wherein adjusting one or more of the first force, the second force and/or the third force includes populating a model stored in a memory of a controller with the one or more parameters; and
wherein output from the model comprises instructions for adjusting one or more of a magnitude and/or a direction of the first force, the second force and/or the third force to satisfy the model.

Additional Aspect 15: The method of additional aspect 11, wherein applying one or more of the first force, the second force and the third force includes actuating one or more motors associated with the robotic support system to control tension in one or more cables coupled to the subject.

Additional Aspect 16: The method of additional aspect 11, further comprising applying neuromodulation to the subject during the rehabilitation training routine, where neuromodulation includes one or more of providing electrical stimulation to the subject and/or providing pharmacological stimulation to the subject.

Additional Aspect 17: A system for controlling a robotic support structure for a subject, comprising:
a plurality of cables configured to apply a first force, a second force and/or a third force on a subject;
one or more motorized actuators for controlling tension in the plurality of cables;
an inertial measurement unit for measuring forces exerted on the subject via the plurality of cables and forces exerted on the plurality of cables via the subject;
a physiological recording unit, configured to monitor one or more of kinematic, kinetic and/or electromyographic activity from the subject; and
a controller, storing instructions in non-transitory memory that, when executed, cause the controller to:
determine the first force, the second force and the third force to apply to the subject based on a selected rehabilitation training routine;
apply the first force, the second force, and the third force to the subject performing the rehabilitation training routine;
monitor one or more of kinematic activity, kinetic activity and/or electromyographic activity from the subject; and
adjust one or more of the first force, the second force, and/or the third force based on one or more of forces exerted on the subject via the plurality of cables or forces the subject exerts on the plurality of cables, kinematic activity from the subject, kinetic activity from the subject and/or electromyographic activity from the subject while the subject is performing the rehabilitation training routine.

Additional Aspect 18: The system of additional aspect 17, further comprising:
a device for providing electrical stimulation to the subject; and
wherein the controller stores additional instructions to apply electrical stimulation to a spinal cord of the subject while the subject is performing the rehabilitation training routine.

Additional Aspect 19: The system of additional aspect 17, wherein the controller stores additional instructions to command the one or more motorized actuators to control tension in the plurality of cables such that one or more of the first force, the second force and/or the third force are held substantially constant while the subject is performing the rehabilitation training routine.

Additional Aspect 20: The system of additional aspect 17, wherein the controller stores additional instructions to command the one or more motorized actuators to control tension in the plurality of cables based on a model of movement specific to the rehabilitation training routine, where the model includes inputs comprising one or more of the forces exerted on the subject via the plurality of cables or forces the subject exerts on the plurality of cables, kinematic activity from the subject, kinetic activity from the subject and/or electromyographic activity from the subject while the subject is performing the rehabilitation training routine.

Additional Aspect 21: The method of additional aspect 1, wherein the forward force is a function of an upward force, the upward force being said first force and the forward force being said second force, and the movement of the subject.

Additional Aspect 22: The method of additional aspect 1, wherein a forward force being said first force and an upward being seid second force, wherein the forward force and the upward force are applied in such a way that there is not net energy transmitted to the user, such that the overall system itself behaves passively.

### REFERENCES AND NOTES

1. G. A. Cavagna, P. A. Willems, N. C. Heglund, Walking on Mars. Nature 393, 636. (1998).
2. A. A. Biewener, Biomechanics of mammalian terrestrial locomotion. Science 250, 1097-1103. (1990).
3. M. H. Dickinson, C. T. Farley, R. J. Full, M. A. Koehl, R. Kram, S. Lehman, How animals move: an integrative view. Science 288, 100-106. (2000).
4. N. E. Thompson, B. Demes, M. C. O'Neill, N. B. Holowka, S. G. Larson, Surprising trunk rotational capabilities in chimpanzees and implications for bipedal walking proficiency in early hominins. Nature communications 6, 8416 (2015).
5. G. A. Cavagna, N. C. Heglund, C. R. Taylor, Mechanical work in terrestrial locomotion: two basic mechanisms for minimizing energy expenditure. Am J Physiol 233, R243-261. (1977).
6. R. M. Alexander, Optimization and gaits in the locomotion of vertebrates. Physiol Rev 69, 1199-1227. (1989).
7. R. W. Selles, J. B. Bussmann, R. C. Wagenaar, H. J. Stam, Comparing predictive validity of four ballistic swing phase models of human walking. J Biomech 34, 1171-1177. (2001).
8. L. Bianchi, D. Angelini, G. P. Orani, F. Lacquaniti, Kinematic coordination in human gait: relation to mechanical energy cost. J Neurophysiol 79, 2155-2170. (1998).
9. F. Lacquaniti, Y. P. Ivanenko, M. Zago, Patterned control of human locomotion. The Journal of physiology 590, 2189-2199 (2012).
10. C. Capaday, The special nature of human walking and its neural control. Trends in neurosciences 25, 370-376. (2002).
11. D. Sutherland, R. Olshen, E. Biden, M. Wyatt, in Clinics in Devel. Med. 104/105. (Mackeith Press: Oxford Blackwell Scientific Pubs, Philadelphia, Pa., 1988), pp. 1Y277.
12. S. Harkema, A. Behrman, H. Barbeau, Evidence-based therapy for recovery of function after spinal cord injury. Handbook of clinical neurology 109, 259-274 (2012).
13. P. Sale, M. Franceschini, A. Waldner, S. Hesse, Use of the robot assisted gait therapy in rehabilitation of users with stroke and spinal cord injury. European journal of physical and rehabilitation medicine 48, 111-121 (2012).
14. L. Awai, M. Bolliger, A. R. Ferguson, G. Courtine, A. Curt, Influence of Spinal Cord Integrity on Gait Control in Human Spinal Cord Injury. Neurorehabil Neural Repair, (2015).
15. G. Hornby, D. Campbell, D. Zemon, J. Kahn, Clinical and Quantitative Evaluation of Robotic-Assisted Treadmill Walking to Retrain Ambulation After Spinal Cord Injury. Topics in Spinal Cord Injury Rehabilitation 11, 1-17 (2005).
16. D. D. Straube, C. L. Holleran, C. R. Kinnaird, A. L. Leddy, P. W. Hennessy, T. G. Hornby, Effects of dynamic stepping training on nonlocomotor tasks in individuals poststroke. Phys Ther 94, 921-933 (2014).
17. E. Swinnen, J. P. Baeyens, S. Pintens, J. Van Nieuwenhoven, S. Ilsbroukx, R. Clijsen, R. Buyl, M. Goossens, R. Meeusen, E. Kerckhofs, Trunk muscle activity during walking in persons with multiple sclerosis: the influence of body weight support. NeuroRehabilitation 34, 323-335 (2014).
18. A. Pennycott, H. Vallery, D. Wyss, M. Spindler, A. Dewarrat, R. Riener, A novel body weight support system extension: initial concept and simulation study. IEEE . . . International Conference on Rehabilitation Robotics: [proceedings] 2013, U.S. Pat. No. 6,650,489 (2013).
19. A. Thorstensson, J. Nilsson, H. Carlson, M. R. Zomlefer, Trunk movements in human locomotion. Acta Physiol Scand 121, 9-22 (1984).
20. N. Dominici, U. Keller, H. Vallery, L. Friedli, R. van den Brand, M. L. Starkey, P. Musienko, R. Riener, G. Courtine, Versatile robotic interface to evaluate, enable and train locomotion and balance after neuromotor disorders. Nature medicine 18, 1142-1147 (2012).
21. W. K. Timoszyk, J. A. Nessler, C. Acosta, R. R. Roy, V. R. Edgerton, D. J. Reinkensmeyer, R. de Leon, Hindlimb loading determines stepping quantity and quality following spinal cord transection. Brain Res 1050, 180-189 (2005).
22. W. Song, S. Giszter, Adaptation to a Cortex-Controlled Robot Attached at the Pelvis and Engaged during Locomotion in Rats. Journal of Neuroscience 31, 3110-3128 (2011).
23. R. van den Brand, J. Heutschi, Q. Barraud, J. DiGiovanna, K. Bartholdi, M. Huerlimann, L. Friedli, I. Vollenweider, E. M. Moraud, S. Duis, N. Dominici, S. Micera, P. Musienko, G. Courtine, Restoring voluntary control of locomotion after paralyzing spinal cord injury. Science 336, 1182-1185 (2012).
24. H. Vallery, P. lutz, J. von Zitzewitz, G. Rauter, M. Fritschi, C. Everarts, R. Ronsse, A. Curt, M. Bolliger, S. B. Pai, in International Conference on Rehabilitation Robotics (ICORR), IEEE, Ed. (Seattle, USA, 2013).
25. C. A. Angeli, V. R. Edgerton, Y. P. Gerasimenko, S. J. Harkema, Altering spinal cord excitability enables voluntary movements after chronic complete paralysis in humans. Brain: a journal of neurology, (2014).
26. J. von Zitzewitz, L. Asboth, N. Fumeaux, A. Hasse, L. Baud, H. Vallery, G. Courtine, A neurorobotic platform for locomotor prosthetic development in rats and mice. J Neural Eng 13, 026007 (2016).
27. L. Friedli, E. S. Rosenzweig, Q. Barraud, M. Schubert, N. Dominici, L. Awai, J. L. Nielson, P. Musienko, Y. Nout-Lomas, H. Zhong, S. Zdunowski, R. R. Roy, S. C. Strand, R. van den Brand, L. A. Havton, M. S. Beattie, J. C. Bresnahan, E. Bezard, J. Bloch, V. R. Edgerton, A. R. Ferguson, A. Curt, M. H. Tuszynski, G. Courtine, Pronounced species divergence in corticospinal tract reorganization and functional recovery after lateralized spinal cord injury favors primates. Science translational medicine 7, 302ra134 (2015).
28. Y. P. Ivanenko, N. Dominici, G. Cappellini, B. Dan, G. Cheron, F. Lacquaniti, Development of pendulum mechanism and kinematic coordination from the first unsupported steps in toddlers. J Exp Biol 207, 3797-3810 (2004).
29. E. M. Moraud, M. Capogrosso, E. Formento, N. Wenger, J. DiGiovanna, G. Courtine, S. Micera, Mechanisms Underlying the Neuromodulation of Spinal Circuits for Correcting Gait and Balance Deficits after Spinal Cord Injury. Neuron 89, 814-828 (2016).
30. N. Wenger, E. M. Moraud, J. Gandar, P. Musienko, M. Capogrosso, L. Baud, C. G. Le Goff, Q. Barraud, N. Pavlova, N. Dominici, I. R. Minev, L. Asboth, A. Hirsch, S. Duis, J. Kreider, A. Mortera, O. Haverbeck, S. Kraus, F. Schmitz, J. DiGiovanna, R. van den Brand, J. Bloch, P. Detemple, S. P. Lacour, E. Bezard, S. Micera, G. Courtine, Spatiotemporal neuromodulation therapies engaging muscle synergies improve motor control after spinal cord injury. Nature medicine 22, 138-145 (2016).
31. Y. P. Ivanenko, N. Dominici, F. Lacquaniti, Development of independent walking in toddlers. Exerc Sport Sci Rev 35, 67-73 (2007).
32. G. Courtine, J. Bloch, Defining ecological strategies in neuroprosthetics. Neuron 86, 29-33 (2015).
33. D. Barthelemy, H. Leblond, S. Rossignol, Characteristics and mechanisms of locomotion induced by intraspinal microstimulation and dorsal root stimulation in spinal cats. Journal of neurophysiology 97, 1986-2000 (2007).
34. G. Courtine, Y. Gerasimenko, R. van den Brand, A. Yew, P. Musienko, H. Zhong, B. Song, Y. Ao, R. M. Ichiyama, I. Lavrov, R. R. Roy, M. V. Sofroniew, V. R. Edgerton, R. V. D. Brand, Transformation of nonfunctional spinal circuits into functional states after the loss of brain input. Nature neuroscience 12, 1333-1342 (2009).
35. B. J. Holinski, D. G. Everaert, V. K. Mushahwar, R. B. Stein, Real-time control of walking using recordings from dorsal root ganglia. J Neural Eng 10, 056008 (2013).
36. N. Wenger, E. M. Moraud, S. Raspopovic, M. Bonizzato, J. DiGiovanna, P. Musienko, M. Morari, S. Micera, G. Courtine, Closed-loop neuromodulation of spinal sensorimotor circuits controls refined locomotion after complete spinal cord injury. Science translational medicine 6, 255ra133 (2014).
37. L. Guevremont, J. A. Norton, V. K. Mushahwar, Physiologically based controller for generating overground locomotion using functional electrical stimulation. J Neurophysiol 97, 2499-2510 (2007).
38. M. R. Carhart, J. He, R. Herman, S. D'Luzansky, W. T. Willis, Epidural spinal-cord stimulation facilitates recovery of functional walking following incomplete spinal-cord injury. IEEE Trans Neural Syst Rehabil Eng 12, 32-42 (2004).
39. Y. Gerasimenko, D. Lu, M. Modaber, S. Zdunowski, P. Gad, D. Sayenko, E. Morikawa, P. Haakana, A. R. Ferguson, R. R. Roy, V. R. P. D. Edgerton, Noninvasive Reactivation of Motor Descending Control after Paralysis. J Neurotrauma, (2015).
40. R. Herman, J. He, S. D'Luzansky, W. Willis, S. Dilli, Spinal cord stimulation facilitates functional walking in a chronic, incomplete spinal cord injured. Spinal Cord 40, 65-68 (2002).
41. S. M. Danner, U. S. Hofstoetter, B. Freundl, H. Binder, W. Mayr, F. Rattay, K. Minassian, Human spinal locomotor control is based on flexibly organized burst generators. Brain: a journal of neurology, (2015).
42. U. S. Hofstoetter, S. M. Danner, B. Freundl, H. Binder, W. Mayr, F. Rattay, K. Minassian, Periodic modulation of repetitively elicited monosynaptic reflexes of the human lumbosacral spinal cord. J Neurophysiol, jn 00136 02015 (2015).
43. M. Wessels, C. Lucas, I. Eriks, S. de Groot, Body weight-supported gait training for restoration of walking in people with an incomplete spinal cord injury: a systematic review. J Rehabil Med 42, 513-519 (2010).
44. K. Musselman, K. Brunton, T. Lam, J. Yang, Spinal cord injury functional ambulation profile: a new measure of walking ability. Neurorehabil Neural Repair 25, 285-293 (2011).
45. D. J. Reinkensmeyer, D. Aoyagi, J. L. Emken, J. A. Galvez, W. Ichinose, G. Kerdanyan, S. Maneekobkunwong, K. Minakata, J. A. Nessler, R. Weber, R. R. Roy, R. de Leon, J. E. Bobrow, S. J. Harkema, V. R. Edgerton, Tools for understanding and optimizing robotic gait training. Journal of rehabilitation research and development 43, 657-670 (2006).
46. L. Ada, C. M. Dean, J. Vargas, S. Ennis, Mechanically assisted walking with body weight support results in more independent walking than assisted overground walking in non-ambulatory users early after stroke: a systematic review. Journal of physiotherapy 56, 153-161 (2010).
47. V. R. Edgerton, R. R. Roy, Robotic training and spinal cord plasticity. Brain Res Bull 78, 4-12 (2009).
48. G. Kwakkel, B. J. Kollen, H. I. Krebs, Effects of robot-assisted therapy on upper limb recovery after stroke: a systematic review. Neurorehabil Neural Repair 22, 111-121 (2008).
49. G. Courtine, Y. Gerasimenko, R. van den Brand, A. Yew, P. Musienko, H. Zhong, B. Song, Y. Ao, R. M. Ichiyama, I. Lavrov, R. R. Roy, M. V. Sofroniew, V. R. Edgerton, Transformation of nonfunctional spinal circuits into functional states after the loss of brain input. Nat Neurosci 12, 1333-1342 (2009).
50. S. Harkema, Y. Gerasimenko, J. Hodes, J. Burdick, C. Angeli, Y. Chen, C. Ferreira, A. Willhite, E. Rejc, R. G. Grossman, V. R. Edgerton, Effect of epidural stimulation of the lumbosacral spinal cord on voluntary movement, standing, and assisted stepping after motor complete paraplegia: a case study. Lancet 377, 1938-1947 (2011).
51. P. K. Shah, G. Garcia-Alias, J. Choe, P. Gad, Y. Gerasimenko, N. Tillakaratne, H. Zhong, R. R. Roy, V. R. Edgerton, Use of quadrupedal step training to re-engage spinal interneuronal networks and improve locomotor function after spinal cord injury. Brain: a journal of neurology 136, 3362-3377 (2013).
52. V. Dietz, Quadrupedal coordination of bipedal gait: implications for movement disorders. J Neurol 258, 1406-1412 (2011).
53. A. Duschau-Wicke, A. Caprez, R. Riener, User-cooperative control increases active participation of individuals with SCI during robot-aided gait training. J Neuroeng Rehabil 7, 43 (2010).
54. G. Demeurisse, A. Capon, M. Verhas, E. Attig, Pathogenesis of aphasia in deep-seated lesions: likely role of cortical diaschisis. Eur Neurol 30, 67-74 (1990).
55. R. A. Keith, C. V. Granger, B. B. Hamilton, F. S. Sherwin, The functional independence measure: a new tool for rehabilitation. Adv Clin Rehabil 1, 6-18 (1987).
56. H. Vallery, P. Lutz, J. Von Zitzewitz, G. Rauter, M. Fritschi, C. Everarts, R. Ronsse, A. Curt, M. Bolliger, in Rehabilitation Robotics (ICORR), 2013 IEEE International Conference on. (IEEE, 2013), pp. 1-7.
57. J. von Zitzewitz, A. Morger, G. Rauter, L. Marchal-Crespo, F. Crivelli, D. Wyss, T. Bruckmann, R. Riener, A reconfigurable, tendon-based haptic interface for research into human-environment interactions. Robotica 31, 441-453 (2012).
58. J. B. Mignardot, O. Beauchet, C. Annweiler, C. Cornu, T. Deschamps, Postural sway, falls, and cognitive status: a cross-sectional study among older adults. Journal of Alzheimer's disease: JAD 41, 431-439 (2014).
59. J. Rummel, Y. Blum, A. Seyfarth, Robust and efficient walking with spring-like legs. Bioinspiration & biomimetics 5, 046004 (2010).

1. An apparatus comprising a robotic support system for a user, said apparatus comprising a controller for said robotic support system, said apparatus comprising: the robotic support system adapted to apply one or more of a z-direction force F_{zsup}, x-direction force Fₓₛᵤₚ and y-direction force F_{ysup}, or any combination thereof, on said user according to the following respective equations:
F_{zsup}=Fz(x,dx/dt,y,dy/dt,z,dz/dt);
Fₓₛᵤₚ=Fx(x,dx/dt,y,dy/dt,z,dz/dt);
F_{ysup}=Fy(x,dx/dt,y,dy/dt,z,dz/dt);
wherein
Fₓₛᵤₚ is the force applied in forward direction,
F_{ysup} is the force applied in lateral direction,
F_{zsup} is the force applied in upward direction, where x, y, and z are the forward, lateral, and vertical coordinate positions of the center of mass in a coordinate system that is fixed to the stance foot and rotates with the person, and dx/dt, dy/dt, dz/dt are the derivatives with respect to time.

2. The apparatus of claim 1, wherein the apparatus is a robotic platform that assists trunk movements in order to optimize gravity-dependent movements and/or gait interactions.

3. The apparatus of claim 1, wherein the controller is configured and arranged such that the controller is capable to perform an algorithm to automatically configure the one- or multidirectional forces applied to the trunk based on user-specific needs.

4. The apparatus of claim 1, wherein said robotic support system applies said upward force F_{zsup} according to the following equation:
F_{zsup}=c_{z}·(z₀−z)+Δm·g,
wherein
c_{z} is the stiffness, which is chosen such that said user walks with a frequency of natural walking;
z is the vertical position of the center of mass of said user;
z₀ is the average or nominal walking height;
Δm is the part of the mass of said user that is compensated by said upward force;
g is gravity acceleration; and
Fₓₛᵤₚ and F_{ysup} are nul.

5. The apparatus of claim 1, wherein said robotic support system applies said forward force Fₓₛᵤₚ according to the following equation:
Fₓₛᵤₚ=cₓₛ·sin(a_{z}·dz/dt) for z≤z₀,
Fₓₛᵤₚ=0 for z>z₀,
wherein
a_{z} and cₓₛ are positive constants,
z₀ is the average or nominal walking height;
or according to the following equation:
Fₓₛᵤₚ=−cₓ·x,
wherein cx is a positive constant;
or according to the following functions:
Fₓₛᵤₚ=Fₓₛᵤₚ(z,dz/dt)
or
Fₓₛᵤₚ=Fₓₛᵤₚ(x).

6. The apparatus of claim 1, wherein said robotic support system applies said lateral force F_{ysup} according to the following equation:
F_{ysup}=c_{y}·y,
wherein c_{y} is stiffness.

7. The apparatus of claim 1, wherein said controller is passive.

8. The apparatus of claim 5, wherein said robotic support system applies said upward force according to the following equation:
F_{zsup}=F_{zsup}(Fₓₛᵤₚ,dx/dt,dz/dt),
whereby F_{zsup} suffices the following inequality constraint:
F_{zsup}<−Fₓₛᵤₚ(dx/dt)/(dz/dt);
or said forward force according to the following equation:
$${F}_{xsup} = \begin{matrix}\{ {F}_{xsup} ( z , dz / dt ) & for dz / dt < 0 and z < {z}_{0}\end{matrix}$$
$$\begin{matrix}\{ {F}_{xsup} ( x ) & for dz / dt > 0 and z < {z}_{0}\end{matrix}$$
$$\begin{matrix}\{ 0 & for z > {z}_{0} .\end{matrix}$$

9. The apparatus of claim 1, further comprising one or more sensors positioned on or in contact with the user for measuring the shift of the mean antero-posterior position of the center of plantar pressure of said user and wherein the robotic support system is further adapted to apply forward force to said user in order to compensate said shift.

10. The apparatus of claim 1, wherein the controller is configured to:
set the apparatus in transparent mode;
compute parameters from kinematic recordings of locomotor tasks performed by said user to obtain and optionally storing a dataset; and
elaborate said dataset with principal component (PC) analysis.

11. The apparatus of claim 9, wherein said controller utilizes an artificial neural network for measuring the shift of the mean antero-posterior position of the center of plantar pressure of said user and for applying forward force to said user in order to compensate said shift.

12. The apparatus of claim 1, wherein said apparatus is provided with a recording platform for real-time acquisition of apparatus-user interactions.

13. The apparatus of claim 1, wherein said apparatus is selected from the group consisting of cable robot, trunk support, exoskeleton, wearable exoskeleton and exosuit.

14. The apparatus of claim 1, further comprising a device for epidural or subdural electrical stimulation.

15. The apparatus of claim 1, wherein for standing and walking of the user the apparatus provides at least an upward force and a forward force to the trunk of the body of the user in order to restore the postural orientation of the body of the user.
